(19) United States
(12) Patent Application Publication
(10) Pub. No.: US 2018/0369287 A1
(43) Pub. Date: Dec. 27, 2018

(54) NEURAL PRECURSOR CELL POPULATIONS AND USES THEREOF

(71) Applicant: Neurona Therapeutics Inc., South San Francisco, CA (US)

(72) Inventors: Cory Nicholas, San Francisco, CA (US); Luis Fuentealba, South San Francisco, CA (US); Cheuk Ka Tong, South San Francisco, CA (US); Marina Bershteyn, South San Francisco, CA (US); Sonja Kriks, South San Francisco, CA (US); Stuart Chambers, South San Francisco, CA (US)

(21) Appl. No.: 15/766,792

(22) PCT Filed: Oct. 10, 2016
(86) PCT No.: PCT/US16/56316

§ 371 Date: Apr. 6, 2018

Related U.S. Application Data
(60) Provisional application No. 62/239,042, filed on Oct. 8, 2015.

(51) Int. Cl. A61K 35/30; C12N 5/0797; C12N 5/0793

(52) U.S. Cl. CPC A61K 35/30; C12N 5/0619; C12N 5/0623

(57) ABSTRACT

The present invention provides cell populations enriched for specific neural precursor markers and methods of using such cell populations for treatment of disorders associated with dysregulation of inhibitory neuronal function and/or imbalances in excitatory/inhibitory neuronal activity. In particular, the present invention provides cell populations for use as a cell-based therapeutic, and methods for purification and use of these neural precursor cells in transplantation to ameliorate neural disorders associated with aberrant neural function.

### CROSS REFERENCE TO RELATED APPLICATIONS

This application claims priority to U.S. Provisional Application 62/239,042, entitled “Neural Precursor Cell Populations and Uses Thereof”, filed Oct. 8, 2015, which is hereby incorporated by reference in its entirety for all purposes.

### FIELD OF THE INVENTION

The present invention relates generally to the fields of cell biology, pluripotent stem cells, and cell differentiation. The invention discloses populations of neural precursor cells and therapeutic uses thereof.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Clinical management of conditions, diseases and injuries of the central and peripheral nervous system remains an area of significant unmet clinical need. The therapies currently used for various disorders, including seizure disorders, Parkinson's disease, traumatic brain injury, pain and spasticity, usually focus on the management of the symptoms rather than addressing the root cause of the disease or disorder. Thus, there remains a pressing need for improved and effective treatments of the central and peripheral nervous system that are able to repair or replace damaged or injured neural tissue.

The present invention addresses this need by providing novel neural precursor cell populations with the ability to migrate and differentiate into functional neurons in vivo.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides cell populations enriched for specific neural precursor markers and methods of using such cell populations for treatment of disorders associated with dysregulation of inhibitory neuronal function and/or imbalances in excitatory/inhibitory neuronal activity. In particular, the present invention provides cell populations for use as cell-based therapeutics, and methods for purification and use of these neural precursor cells in transplantation to ameliorate neural disorders associated with aberrant neural function.

Thus, in one embodiment, the invention provides enriched populations of neural precursor cells that express key factors that indicate the ability of these cells to efficiently differentiate into inhibitory interneurons upon transplantation into a mammal. Preferably, the neural precursor cells are enriched in expression of markers expressed by cortical interneurons, cells that predominantly originate in the MGE. The cell populations of the invention may be enriched using methods including but not limited to: isolation using cell surface markers; depletion of cell populations using cell surface markers downregulated in neural precursors; and differentiation of pluripotent cells to express neural precursor markers, etc.

Exemplary neural precursor cell markers enriched in the population include, but are not limited to, AS1, ATRNL1, CD200, CELSR3, CHRM4, CNTNAP4, CXCR4, CXCR7, DSCAML1, ELAVL2, ENSG00000260391, EPHA5, ERBB4, FAM5B, FAM65B, FNDC5, GAD1, GAD2, GNG2, GPD1, GRIA1, GRIA4, HMP19, INA, KALRN, KDM6B, KIF21B, L1CAM, LHX6, LINC00340, LINC00599, MAF, MAFB, MAPT, MIAT, NCAM1, NKX2-1, NMNAT2, NPAS1, NRCAM, NRXN3, NXPH1, PDZRN4, PIP5K1B, PLS3, PLXNA4, RAI2, ROBO1, ROBO2, RP11-384F7.2, RP4-791M13.3, RUNX1T1, SCG3, SCRT1, SCRT2, SIAH3, SLC32A1, SOX6, SRRM4, SST, ST8SIA5, STMN2, TAGLN3, TIAM1, TMEM2, TTC9B, or WI2-1896O14.1.

In some embodiments, the invention provides a neural precursor cell population comprising cells capable of differentiating into GABA-expressing cells, wherein the cell population comprises a majority of cells (50% or more) that express one or more of the neural precursor markers AS1, ATRNL1, CD200, CELSR3, CHRM4, CNTNAP4, CXCR4, CXCR7, DSCAML1, ELAVL2, ENSG00000260391, EPHA5, ERBB4, FAM5B, FAM65B, FNDC5, GAD1, GAD2, GNG2, GPD1, GRIA1, GRIA4, HMP19, INA, KALRN, KDM6B, KIF21B, L1CAM, LHX6, LINC00340, LINC00599, MAF, MAFB, MAPT, MIAT, NCAM1, NKX2-1, NMNAT2, NPAS1, NRCAM, NRXN3, NXPH1, PDZRN4, PIP5K1B, PLS3, PLXNA4, RAI2, ROBO1, ROBO2, RP11-384F7.2, RP4-791M13.3, RUNX1T1, SCG3, SCRT1, SCRT2, SIAH3, SLC32A1, SOX6, SRRM4, SST, ST8SIA5, STMN2, TAGLN3, TIAM1, TMEM2, TTC9B, or WI2-1896O14.1. In some aspects, the neural precursor cells can differentiate to form neurons capable of producing GABA in vitro. In other aspects, the neural precursor cells can differentiate to form neurons capable of producing GABA following transplantation into a mammalian nervous system (e.g., the central nervous system, or CNS).

The neural precursor cell populations of the invention can be isolated from human tissue (e.g., human fetal cortex or human ganglionic eminences), or can be differentiated from stem cells or other multipotent cells. Thus, in some embodiments, the neural precursor cell populations are isolated from a source of pluripotent stem cells. In some embodiments, the neural precursor cells are differentiated from human stem cells, e.g., human embryonic stem cells. In other embodiments, the neural precursor cells are differentiated from induced pluripotent stem cells. In yet other embodiments, the neural precursor cells are differentiated from neural stem cells. In yet other embodiments, the neural precursor cell populations are created through reprogramming of cells, e.g., neural cells obtained from the MGE, Cortex, Sub-Cortex, other regions of the brain, or non-neural cells.

Thus, in a specific embodiment, the invention provides a method of generating a population of neural precursor cells, comprising isolating cells from mammalian brain tissue under conditions to allow the cells to increase expression of one or more cell-surface markers upregulated in neural precursor cells, and enriching the neural cell-surface marker-expressing cells to generate a population of cell surface marker enriched cells, wherein the enriched cell population comprises neural precursor cells capable of forming GABA-producing neurons in vitro and/or upon transplantation into a mammalian nervous system (e.g., the CNS). In preferred embodiments the cell-surface marker is ATRNL1, CD200, CELSR3, CHRM4, CNTNAP4, CXCR4, CXCR7, DSCAML1, EPHA5, ERBB4, FAM5B, FAM65B, FNDC5, GRIA1, GRIA4, L1CAM, NCAM1, NRCAM, NRXN3, NXPH1, PLXNA4, ROBO1, ROBO2, or TMEM2.

In other specific embodiments, the invention provides a method of generating a population of neural precursor cells, comprising providing a population of pluripotent mammalian stem cells; differentiating the stem cells under conditions to allow the cells to increase expression of one or more cell-surface markers upregulated in neural precursor cells of interest; and enriching the cell population for cells expressing one or more of said cell surface markers; wherein the enriched cell population comprises neural precursor cells capable of forming GABA-producing neurons in vitro and/or upon transplantation into a mammalian brain. Preferably, the neural precursor cell surface marker is ATRNL1, CD200, CELSR3, CHRM4, CNTNAP4, CXCR4, CXCR7, DSCAML1, EPHA5, ERBB4, FAM5B, FAM65B, FNDC5, GRIA1, GRIA4, L1CAM, NCAM1, NRCAM, NRXN3, NXPH1, PLXNA4, ROBO1, ROBO2, or TMEM2.

In some embodiments, the enriched cells are also enriched in expression of a second neural precursor cell marker. For example, in addition to the cell surface marker used to enrich the cells, the cells may be further enriched to express one or more of AS1, ATRNL1, CD200, CELSR3, CHRM4, CNTNAP4, CXCR4, CXCR7, DSCAML1, ELAVL2, ENSG00000260391, EPHA5, ERBB4, FAM5B, FAM65B, FNDC5, GAD1, GAD2, GNG2, GPD1, GRIA1, GRIA4, HMP19, INA, KALRN, KDM6B, KIF21B, L1CAM, LHX6, LINC00340, LINC00599, MAF, MAFB, MAPT, MIAT, NCAM1, NKX2-1, NMNAT2, NPAS1, NRCAM, NRXN3, NXPH1, PDZRN4, PIP5K1B, PLS3, PLXNA4, RAI2, ROBO1, ROBO2, RP11-384F7.2, RP4-791M13.3, RUNX1T1, SCG3, SCRT1, SCRT2, SIAH3, SLC32A1, SOX6, SRRM4, SST, ST8SIA5, STMN2, TAGLN3, TIAM1, TMEM2, TTC9B, or WI2-1896O14.1.

In some embodiments, the cell-surface marker-expressing cells are enriched using an agent (e.g., an antibody) that binds selectively to a neural precursor cell surface marker. In specific embodiments, the neural precursor cell-surface marker-expressing cells are isolated by a fluorescence-activated cell sorting (FACS). In other specific embodiments, the neural precursor cell-surface marker-expressing cells are isolated using magnetic-activated cell sorting (MACS).

Preferably, the neural precursor cells are capable of forming functional inhibitory interneurons that integrate into the central or peripheral nervous system of a mammal following transplantation, and such formation and integration of the functional inhibitory neurons is associated with the treatment of a neural disorder.

In another aspect, the invention features a method for isolating a population of neural precursor cells of the invention. The method includes the steps of providing a tissue from a subject (e.g., tissue from a fetal mammalian brain) or cells differentiated from a pluripotent cell source and enriching the selected cell population using one or more different cell surface proteins selected from ATRNL1, CD200, CELSR3, CHRM4, CNTNAP4, CXCR4, CXCR7, DSCAML1, EPHA5, ERBB4, FAM5B, FAM65B, FNDC5, GRIA1, GRIA4, L1CAM, NCAM1, NRCAM, NRXN3, NXPH1, PLXNA4, ROBO1, ROBO2, or TMEM2, thereby isolating a population of neural precursor cells.

In another aspect, the invention features a method for depleting the isolated cell populations from unwanted cells using one or more cell surface proteins which have at least a two-fold suppression in the neural precursor cells of the invention. For example, neural precursor cell populations can be enriched by depletion of a cell population with using one or more different cell surface proteins selected from ATP1A2, BCAN, CD271, CD98, CNTFR, FGFR3, GJA1, MLC1, NOTCH1, NOTCH3, PDPN, PTPRZ1, SLC1A5, TMEM158, or TTYH1.

In addition or alternatively, the method may further include a step of cryopreserving the cells.

The method may further include culturing the population of neural precursor cells under conditions which support proliferation of the cells.

The invention also features a neural precursor cell population produced by any of the above methods.

The invention also provides a population of neural precursor cells comprising a majority of cells (greater than 50%) with the ability to differentiate into a functional inhibitory interneuron upon transplantation to a mammalian central or peripheral nervous system.

The present invention has identified that cells expressing the cell-surface marker PLEXINA4 are enhanced in their ability to mature into functional cortical interneurons upon transplantation into the mammalian CNS. Thus, in certain embodiments, the population of the neural precursor cells expresses PLEXINA4 as one of the enriched neural precursor markers.

In a specific embodiment, the invention provides a population of neural precursor cells, wherein the population is enriched in cells comprising increased expression of one or more of AS1, ATRNL1, CD200, CELSR3, CHRM4, CNTNAP4, CXCR4, CXCR7, DSCAML1, ELAVL2, ENSG00000260391, EPHA5, ERBB4, FAM5B, FAM65B, FNDC5, GAD1, GAD2, GNG2, GPD1, GRIA1, GRIA4, HMP19, INA, KALRN, KDM6B, KIF21B, L1CAM, LHX6, LINC00340, LINC00599, MAF, MAFB, MAPT, MIAT, NCAM1, NKX2-1, NMNAT2, NPAS1, NRCAM, NRXN3, NXPH1, PDZRN4, PIP5K1B, PLS3, PLXNA4, RAI2, ROBO1, ROBO2, RP11-384F7.2, RP4-791M13.3, RUNX1T1, SCG3, SCRT1, SCRT2, SIAH3, SLC32A1, SOX6, SRRM4, SST, ST8SIA5, STMN2, TAGLN3, TIAM1, TMEM2, TTC9B, or WI2-1896O14.1; and increased expression of PLEXINA4. These neural precursor cells are capable of forming GABA-producing neurons in vitro and/or upon transplantation into a mammalian nervous system (e.g., a mammalian CNS).

In another embodiment, the invention provides a population of neural precursor cells, wherein the population is enriched in cells comprising increased expression of one or more cell-surface markers of ATRNL1, CD200, CELSR3, CHRM4, CNTNAP4, CXCR4, CXCR7, DSCAML1, EPHA5, ERBB4, FAM5B, FAM65B, FNDC5, GRIA1, GRIA4, L1CAM, NCAM1, NRCAM, NRXN3, NXPH1, PLXNA4, ROBO1, ROBO2, or TMEM2; and increased expression of PLEXINA4. These neural precursor cells are capable of forming GABA-producing neurons in vitro and/or upon transplantation into a mammalian nervous system (e.g., a mammalian CNS).

In some embodiments, a method is provided for the treatment of a mammal having a neurological condition, disease, or injury associated with inhibitory neuronal dysfunction and/or excitatory-inhibitory imbalance, comprising transplanting a neural precursor cell population of the invention into the nervous system of the mammal. The populations of neural precursor cells of the invention are distinguished by expression of specific signature transcripts and/or lack of expression of other transcripts that identify the cells as migratory cells capable of functionally integrating into the host nervous system, and particularly into the host central nervous system, as described in more detail herein. Neural precursor cells of the invention are able to migrate at least 0.5 mm from the transplantation site, and to mature and functionally integrate into the endogenous tissue at the desired site of treatment.

The neurological conditions, diseases, or injuries amendable to treatment with the methods of the invention include various degenerative diseases, developmental diseases, genetic diseases, acute injuries, and chronic injuries. The cells may be transplanted into the central nervous system or the peripheral nervous system. In some embodiments, the neurological condition, disease, or injury includes, but is not limited to, Parkinson's disease, seizure disorders (e.g., epilepsy), spasticity, spinal cord injury, brain injury, or peripheral nerve damage, pain (e.g., neuropathic pain), Alzheimer's disease, anxiety, autism, stroke, chronic itch, amblyopia/visual plasticity, psychosis (e.g., schizophrenia), dyskinesia and/or dystonia.

Thus, the invention also provides a method for treating a neural disorder in a subject, said method comprising transplanting a population of neural precursor cells into the nervous system of a mammal afflicted with a neural disorder, wherein at least 50% of the population comprises cells enriched for one or more of the transcripts selected from AS1, ATRNL1, CD200, CELSR3, CHRM4, CNTNAP4, CXCR4, CXCR7, DSCAML1, ELAVL2, ENSG00000260391, EPHA5, ERBB4, FAM5B, FAM65B, FNDC5, GAD1, GAD2, GNG2, GPD1, GRIA1, GRIA4, HMP19, INA, KALRN, KDM6B, KIF21B, L1CAM, LHX6, LINC00340, LINC00599, MAF, MAFB, MAPT, MIAT, NCAM1, NKX2-1, NMNAT2, NPAS1, NRCAM, NRXN3, NXPH1, PDZRN4, PIP5K1B, PLS3, PLXNA4, RAI2, ROBO1, ROBO2, RP11-384F7.2, RP4-791M13.3, RUNX1T1, SCG3, SCRT1, SCRT2, SIAH3, SLC32A1, SOX6, SRRM4, SST, ST8SIA5, STMN2, TAGLN3, TIAM1, TMEM2, TTC9B, or WI2-1896O14.1, and allowing the transplanted cells to migrate and integrate in the central nervous system of said mammal, thereby treating the neural disorder in said mammal.

In some embodiments, the neurological condition treated is a seizure disorder (e.g., epilepsy), wherein transplantation of neural precursor cells of the invention result a reduction in spontaneous electrographic seizure activity. In specific embodiments, the neurological condition is epilepsy, wherein transplantation of neural precursor cells of the invention result in a reduction in seizure intensity and/or duration. In some embodiments, the neurological condition is epilepsy, wherein transplantation of neural precursor cells of the invention result in reduction in seizure frequency and/or intensity. In some embodiments, the neurological condition is epilepsy, wherein transplantation of neural precursor cells of the invention result in reduction in required antiepileptic drug use in the patient receiving the transplant.

In some embodiments, the neurological disease treated with the methods of the invention is Parkinson's disease, wherein transplantation of neural precursor cells of the invention result a reduction in required anti-Parkinsonian drug use. In some embodiments, the neurological disease is Parkinson's disease, wherein transplantation of neural precursor cells of the invention result in a reduction in tremor at rest, rigidity, akinesia, bradykinesia, postural instability, flexed posture and/or freezing.

In some embodiments, the neurological condition treated is spasticity, including but not limited to neurogenic bladder spasticity, wherein transplantation of neural precursor cells of the invention mitigates or obviates the need for medication or surgery. In some embodiments, the neurological condition is spasticity, wherein transplantation of neural precursor cells of the invention result in a reduction in required antispasmodic drug use.

In other embodiments, the neurological condition treated using the methods of the invention is nerve injury, (e.g., spinal cord or peripheral nerve injury), wherein transplantation of neural precursor cells of the invention result in improvement of the physiological impairment associated with the nerve injury.

In yet other embodiments, the neurological condition treated is pain, (e.g., chronic pain or neuropathic pain), wherein transplantation of neural precursor cell populations of the invention results in a reduction in pain in the subject treated.

In still other embodiments, the neurological condition treated using the methods of the invention is Alzheimer's Disease, wherein transplantation of neural precursor cell populations of the invention results in an increased capacity for learning and memory.

In still other embodiments, the neurological condition treated using the methods of the invention is traumatic brain injury (e.g., stroke), wherein the transplantation of the neural precursor cell populations of the invention results in an improvement in locomotion and/or coordination.

In yet other embodiments, the neurological conditions treated using the methods of the invention are neuro-developmental or psychiatric diseases, including autism, schizophrenia or psychoses, wherein the transplantation of the neural precursor cell populations of the invention ameliorate behaviors such as social deficits and learning deficiencies in these patients.

In each of the above treatment regimes, the transplantation of the neural precursor cells of the invention results in at least a 10% improvement in disease-associated symptoms in the subject, more preferably at least a 20% improvement in disease-associated symptoms in the subject, even more preferably at least a 30% improvement in disease-associated symptoms in the subject

Preferably, the transplanted neural precursor cells or cells resulting from the transplanted cells survive for at least 1 month, preferably 2 months, and more preferably 6 months following transplantation in the subject.

These aspects and other features and advantages of the invention are described below in more detail. Those skilled in the art will recognize, or be able to ascertain using no more than routine experimentation, many equivalents to the specific embodiments of the invention described herein. Such equivalents are intended to be encompassed by the following claims.

### BRIEF DESCRIPTION OF THE FIGURES

FIG. 1 is a series of graphs illustrating the efficiency of FACS sorting of cortical human interneurons using APC-conjugated anti-CXCR4 antibodies (FIG. 1B) and APC-conjugated anti-ERBB4 antibodies (FIG. 1D), or respective isotype negative control antibodies (FIGS. 1A and 1C).

FIG. 2 is a bar graph illustrating enriched expression by quantitative RTPCR of MGE-specific markers LHX6, DLX2 and SOX6 markers in surface marker positive FACS sorted cell populations compared to respective surface marker negative population controls.

FIG. 3 is a bar graph illustrating largely decreased expression by quantitative RT-PCR of markers of other non-MGE type GABAergic interneuron cell types in the surface marker positive FACS sorted cell populations compared to respective surface marker negative population controls.

FIG. 4 is a series of graphs illustrating flow cytometry analysis of the difference in cellular debris/dead cells (left) and CXCR4 expressing-cell purity (right) in the presorted cell population (top) versus the post-FACS sorted surface marker positive population using APC-conjugated anti-CXCR4 antibodies (bottom).

FIG. 5 is a series of graphs illustrating flow cytometry analysis of the difference in ERBB4-expressing-cell purity in the presorted cell population (top) versus the post-FACS sorted surface marker positive population using APC-conjugated anti-ERRB4 antibodies (bottom).

FIG. 6 is a series of graphs illustrating flow cytometry analysis of the percentage of surface marker positive cells before magnetic MACS sorting (pre-sort, left) and the purity of surface marker positive cells after the use of MACS sorting (post-sort, right) to isolate both MACS positive and MACS negative populations. MACS sorting was performed with anti-ERBB4 biotinylated primary antibodies followed by an anti-biotin secondary antibody conjugated to a magnetic bead. Flow cytometry analysis pre- and post-MACS sort was performed using APC-conjugated anti-ERRB4 antibodies.

FIG. 7 is a bar graph summarizing the reproducible post-sort purity of MACS isolated surface marker positive and negative populations as a percentage of cells expressing the surface marker ERBB4 by post-sort flow cytometry analysis.

FIG. 8 is a bar graph quantifying post-sort protein expression by immunocytochemistry analysis of the isolated surface marker positive population showing enriched expression of exemplary GABAergic interneuron markers (LHX6, DCX, ERBB4) and depleted expression of markers of non-interneuron cell lineages.

FIG. 9 is a table summarizing flow cytometry analysis of pre-sorted neural cell populations showing the percentage of cells expressing various interneuron surface markers.

FIG. 10 is a table of enriched transcript expression by RNA sequencing analysis showing fold changes of the most upregulated transcripts, along with select markers, in the surface marker positive population, compared to the negative population, isolated by FACS using anti-CXCR4 antibodies.

FIG. 11 is a table of enriched transcript expression by RNA sequencing analysis showing fold changes of the most upregulated transcripts, along with select markers, in the surface marker positive population, compared to the negative population, isolated by FACS using anti-CXCR7 antibodies.

FIG. 12 is a table of enriched transcript expression by RNA sequencing analysis showing fold changes of the most upregulated transcripts, along with select markers, in the surface marker positive population, compared to the negative population, isolated by FACS using anti-ERBB4 antibodies.

FIGS. 13A-13C are tables of enriched surface marker transcript expression by RNA sequencing analysis showing the fold changes of the most upregulated surface markers in positive cell populations (compared to respective negative populations) isolated by FACS using anti-CXCR4 antibodies, anti-CXCR7 antibodies, and anti-ERBB4 antibodies.

FIG. 14 is a table of marker sets that can define various cell lineages and the fold changes of these markers, by RNA sequencing, in surface marker positive populations isolated by FACS (compared to respective surface marker negative populations) showing enriched MGE interneuron marker transcript expression and largely depleted expression of transcripts that mark various non-interneuron cell lineages.

FIG. 15 is a set of bar graphs showing HPLC analysis of levels of GABA in collected cell culture media from isolated surface marker positive and negative cell populations sorted by either FACS (left) or MACS (right) that were replated post-sort.

FIG. 16 is a graph showing migration of human HNA+ neuronal precursor cells in the rodent brain at one month post-injection with neural precursor cell surface marker (NPCSM+) positive cells isolated by cell sorting prior to transplantation.

FIG. 17 is a graph showing immunohistochemistry quantification of human HNA+ cells that co-express GABAergic interneuron markers LHX6, CMAF, and MAFB in the rodent brain one month post-injection of neural precursor cell surface marker (NPCSM+) positive cells isolated by cell sorting prior to transplantation.

FIG. 18 is a graph showing immunohistochemistry quantification of human HNA+ cells that co-express markers of cortical interneuron subtype maturation, SST and CALR, in the rodent brain at 90 days and 130 days post-injection with neural precursor cell surface marker (NPCSM+) positive cells isolated by cell sorting prior to transplantation.

FIG. 19 is a schematic showing sites of injection in the adult rodent brain.

FIG. 20A shows a graph illustrating three populations isolated by FACS sorting based on the expression of surface markers PLXNA4 alone, or PLXNA4 and one other NPCSM, and FIGS. 20A and 20B include bar graphs showing quantitative RTPCR analysis of the isolated populations. Isolated surface marker positive populations show enrichment of GABAergic interneuron marker transcripts, and depletion of non-interneuron markers (OLIG2, ISL1, CHAT), compared to surface marker negative populations.

FIG. 21A shows a graph illustrating three populations isolated from human ESC-derived neural precursor cell cultures by FACS sorting based on the expression of surface markers NPCSM alone, or PLXNA4 and one other NPCSM, and FIGS. 21A and 21B include bar graphs showing quantitative RTPCR analysis of the isolated populations. Isolated surface marker positive populations show enrichment of GABAergic interneuron marker transcripts, and depletion of non-interneuron markers (OLIG2, ISL1, CHAT, LHX8, GBX1, ZIC1), compared to surface marker negative populations.

FIG. 22 is a table of RNA sequencing analysis showing fold changes of exemplary transcripts upregulated in PLEXINA4+ NPCSM+ cells over PLEXINA4−NPCSM− cell populations isolated by FACS sorting.

FIG. 23 is a table of RNA sequencing analysis showing fold changes of exemplary transcripts downregulated in PLEXINA4+ NPCSM+ cells over PLEXINA4− NPCSM− cell populations isolated by FACS sorting.

FIG. 24 is a table of RNA sequencing analysis showing fold changes of exemplary transcripts upregulated in PLEXINA4+ NPCSM+ cells over PLEXINA4+ NPCSM− cell populations isolated by FACS sorting.

FIG. 25 is a table of RNA sequencing analysis showing fold changes of exemplary transcripts downregulated in PLEXINA4+ NPCSM+ cells over PLEXINA4+ NPCSM− cell populations isolated by FACS sorting.

FIG. 26 is a table of RNA sequencing analysis showing fold changes of exemplary transcripts upregulated in PLEXINA4+ NPCSM− cells over PLEXINA4− NPCSM− cell populations isolated by FACS sorting.

FIG. 27 is a table of RNA sequencing analysis showing fold changes of exemplary transcripts downregulated in PLEXINA4+ NPCSM− cells over PLEXINA4− NPCSM− cell populations isolated by FACS sorting.

FIG. 28 is a series of histogram graphs showing flow cytometry analysis of the percentage of NPCSM+ cells pre-sort (unsorted) and post-MACS sorting to isolate NPCSM+ and NPCSM− populations from four different human ESC lines differentiated toward the MGE-type interneuron lineage.

FIG. 29 is a series of bar graphs of immunocytochemistry analysis showing enrichment of cells expressing cortical interneuron marker transcripts, and depletion of other cell types expressing OLIG2, KI67, ISL1, in NPCSM+ populations compared to NPCSM− and unsorted populations isolated by magnetic MACS from four different human ESC lines differentiated toward the MGE-type interneuron lineage.

FIG. 30 is a series of bar graphs of immunohistochemistry analysis and quantification of the percentages of human HNA+ cells co-expressing various markers showing human interneuron maturation in the rodent brain at one and two months post-transplant with NPCSM+ cells sorted from hESC-derived cultures.

FIG. 31 is a graph showing migration of human HNA+ neuronal precursor cells in the rodent brain at one month post-injection with NPCSM+ cells isolated by cell sorting from two different human ESC lines.

FIG. 32 is a graph showing HPLC analysis of levels of GABA in collected cell culture media from sorted PLXNA4 and/or one other NPCSM surface marker positive and negative cell populations isolated from human ESC-derived cultures and replated post-sort.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “isolated” as used herein refers to purification or substantial purification of a cell population that comprises cells with a specific transcript signature, e.g., expression of cells with expression of transcripts that are indicative of the cell's ability to migrate and/or differentiate.

A “stem cell” is commonly defined as a cell that (i) is capable of renewing itself; and (ii) can give rise to more than one type of cell through asymmetric cell division (Watt et al., Science, 284:1427-1430, 2000). Stem cells typically give rise to a type of multipotent cell called a progenitor cell.

A “precursor cell” is a cell capable of differentiating into lineage-committed cells that populate the body. Such cells may be pre- or post-mitotic, and include but are not limited to progenitor cells and cells with an established neural fate that have not fully completed differentiation and/or integration into the endogenous host tissue.

The terms “neural precursor cell” and a “neural precursor cell of interest” as described refer to a cell that capable of migrating and differentiating into a GABA-producing inhibitory interneuron in vitro or in vivo. Such precursor cells of the invention are preferably migratory cells with the ability to migrate from the site of transplantation to the desired site of treatment. Such cells may arise, e.g., from the MGE, CGE, LGE or another part of the mammalian brain. Such cells may also be differentiated from or reprogrammed from other cell types. The neural precursor cells for use in the methods of the invention are further defined by their expression patterns and in vitro and in vivo activities, as described herein in more detail.

### DETAILED DESCRIPTION OF THE INVENTION

The practice of the techniques described herein may employ, unless otherwise indicated, conventional techniques and descriptions of cell biology, cell culture, molecular biology (including recombinant techniques), biochemistry, therapeutic formulations, stem cell differentiation, all of which are within the skill of those who practice in the art. Such conventional techniques include differentiation techniques complementary or useful to the methods described herein; technologies for formulations of therapeutics comprising cell populations, delivery methods that are useful for the delivery of the cell populations of the invention, and the like. Specific illustrations of suitable techniques can be had by reference to the examples herein.

Such conventional techniques and descriptions can be found in standard laboratory manuals such as See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986), all of which are herein incorporated in their entirety by reference for all purposes.

The transcripts and genes as referenced herein are using a naming convention such as that used in the Weitzman Institutes GeneCards® Human Gene Database (http://www.genecards.org/) and/or the databases of the National Center for Biotechnology Information (http:/www.ncbi.nlm.nih.gov) as of the priority and filing dates of the present application.

Note that as used herein and in the appended claims, the singular forms “a,” “an,” and “the” include plural referents unless the context clearly dictates otherwise. Thus, for example, reference to “a cell” refers to one or more cells with various pluripotency and expression patterns, and reference to “the method” includes reference to equivalent steps and methods known to those skilled in the art, and so forth. h

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art upon reading the specification that the present invention may be practiced without one or more of these specific details. In other instances, well-known features and procedures well known to those skilled in the art have not been described in order to avoid obscuring the invention.

The present invention provides populations of neural precursor cells, methods of producing neural precursor cell populations, and methods of treatment using such neural precursor cell populations. A hallmark characteristic of these cells is the capacity to migrate and differentiate into functional inhibitory interneurons in the endogenous tissue of a mammal. Such cell populations can be identified by expression levels of certain signature transcripts or markers indicative of the neural precursor cells. Such cell populations can also be identified by decreased expression levels of other transcripts indicative of other neural cell types. The neural precursor cell populations of the invention have the ability to migrate following transplantation and to differentiate into functional inhibitory interneurons.

The enriched neuronal precursor cell markers will generally display at least two-fold higher levels than other cell types, e.g., astrocytes, endothelial cells, intermediate progenitor cells of excitatory cortical neurons, microglia, excitatory cortical projection neurons, oligodendrocytes, and radial glia progenitors of excitatory cortical neurons. In other embodiments, enriched neuronal precursor cell markers will generally display at least two-fold higher levels of expression of a marker compared to pluripotent cells, e.g., undifferentiated human ES cells.

In some embodiments, the invention provides a population of neural precursor cells, wherein at least 50% of the cell population comprises cells that are enriched in two or more neural precursor cell markers. In other embodiments, the invention provides a population of neural precursor cells, wherein at least 60% of the cell population comprises cells that are enriched in two or more neural precursor cell markers. In certain embodiments, the invention provides a population of neural precursor cells, wherein at least 70% of the cell population comprises cells that are enriched in two or more neural precursor cell markers. In certain other embodiments, the invention provides a population of neural precursor cells, wherein at least 80% of the cell population comprises cells that are enriched in two or more neural precursor cell markers. In yet other embodiments, the invention provides a population of neural precursor cells, wherein at least 90% of the cell population comprises cells that are enriched in two or more neural precursor cell markers.

In other embodiments, the invention provides a population of neural precursor cells, wherein at least 55% of the cell population comprises cells that express at least a two-fold or more increase in expression of neuronal precursor cell markers compared to other neural cell types. In some embodiments, at least 80% of the cell population comprises cells that express at least a two-fold or more increase in expression of neuronal precursor cell markers transcripts compared to other neural cell types. In other specific embodiments, at least 90% of the cell population comprises cells that express at least a two-fold or more increase in expression of neuronal precursor cell markers compared to other neural cell types.

In some preferred embodiments, the expression of the neural precursor cell marker is increased at least 10-fold over the expression in compared to other neural cell types.

In other embodiments, the invention provides a population of neural precursor cells, wherein at least 55% of the cell population expresses two or more, preferably 3 or more, even more preferably 5 or more neural precursor markers indicative of the ability of the cell to migrate and differentiate into an interneuron, and specifically a GABA-expressing interneuron. In some embodiments, at least 70% of the cell population expresses two or more, preferably 3 or more, even more preferably 5 or more neural precursor markers indicative of the ability of the cell to migrate and differentiate into an interneuron, and specifically a GABA-expressing interneuron. In yet other embodiments, at least 80% of the cell population expresses two or more, preferably 3 or more, even more preferably 5 or more neural precursor markers indicative of the ability of the cell to migrate and differentiate into an interneuron, and specifically a GABA-expressing interneuron

Preferably, the neural precursor cell populations of the invention comprise at least 55% neural precursor cells that are capable of efficiently differentiating into inhibitory interneurons upon transplantation into a mammal, more preferably at least 80% neural precursor cells that are capable of efficiently differentiating into inhibitory interneurons upon transplantation into a mammal, more preferably at least 90% neural precursor cells that are capable of efficiently differentiating into inhibitory interneurons upon transplantation into a mammal, and even more preferably at least 95% cells that are capable of efficiently differentiating into inhibitory interneurons upon transplantation into a mammal.

The cells of the invention are uniquely suited for large scale use for various indications, as described in more detail herein. Preferably, at least 50% of the cells of the neural precursor cell population mature into GABAergic inhibitory interneurons upon transplantation into the mammalian central or peripheral nervous system, more preferably at least 60% of the cells of the neural precursor cell population mature into GABAergic inhibitory interneurons upon transplantation into the mammalian central or peripheral nervous system, even more preferably at least 70% of the cells of the neural precursor cell population mature into GABAergic inhibitory interneurons upon transplantation into the mammalian central or peripheral nervous system, still more preferably at least 80% of the cells of the neural precursor cell population mature into GABAergic inhibitory interneurons upon transplantation into the mammalian central or peripheral nervous system, at least 90% of the cells of the neural precursor cell population mature into GABAergic inhibitory interneurons upon transplantation into the mammalian central or peripheral nervous system, still more preferably at least 95% of the cells of the neural precursor cell population mature into GABAergic inhibitory interneurons upon transplantation into the mammalian central or peripheral nervous system.

### Generation of Neural Precursor Cell Populations

In certain embodiments, the neural precursor cell populations of the invention are enriched using one or more cell surface proteins that are expressed on MGE-derived human interneurons. Such markers are more abundantly expressed in human cortical interneurons than in a population of excitatory neurons or other cell types such as radial glia or undifferentiated human pluripotent stem cells. Cell surface markers for use in isolation and/or enrichment of the neural precursor cell populations of the invention include, but are not limited to, ATRNL1, CD200, CELSR3, CHRM4, CNTNAP4, CXCR4, CXCR7, DSCAML1, EPHA5, ERBB4, FAM5B, FAM65B, FNDC5, GRIA1, GRIA4, L1CAM, NCAM1, NRCAM, NRXN3, NXPH1, PLXNA4, ROBO1, ROBO2, or TMEM2.

In other embodiments, the cell population is isolated or enriched using more general neuronal cell surface proteins, and further enriched using one or more specific methods for enrichment of the neural precursor cells as described herein. For example, pan-neuronal markers including, but not limited to CD24, CD56, CD200, L1CAM and NCAM, PSANCAM, may be used to isolate a cell population which is further enriched to provide the neural precursor cells of the invention.

The neural precursor cell populations of the invention may also be isolated and/or enriched using non-antibody based purification methods, preferably in conjunction with another method for enriching the cells to provide a majority of precursor cells with the capacity to differentiate into functional inhibitory interneurons, migrate and/or functionally integrate upon transplantation. Such purification methods include, but are not limited to, size selection (e.g., by density gradient, FACS or MACS), use of labeled ligands to cell surface receptors, or through the use of enhancer-promoter reporter gene expression or use of labeled surface markers.

For example, the cell population may be initially isolated from a source such as fetal neural tissue or cells differentiated from pluripotent or neural stem cells using antibodies against cell surface markers, e.g., ATRNL1, CD200, CELSR3, CHRM4, CNTNAP4, CXCR4, CXCR7, DSCAML1, EPHA5, ERBB4, FAM5B, FAM65B, FNDC5, GRIA1, GRIA4, L1CAM, NCAM1, NRCAM, NRXN3, NXPH1, PLXNA4, ROBO1, ROBO2, or TMEM2. The cell population may then be further enriched using additional cell selection based on neural precursor cell surface markers that are indicative of the ability of the cells to further differentiate into functional inhibitory interneurons.

Methods for isolation of neural precursors from a biological sample include, but are not limited to, cell fractionation by size and density; highly selective affinity-based technologies such as affinity chromatography, fluorescence-activated cell sorting (FACS) and magnetic cell sorting; enhancer-reporter based isolation; tagged ligand based isolation; and isolation based on functional properties of the neural precursor cells. See e.g., Dainiak M B et al., Adv Biochem Eng Biotechnol. 2007; 106:1-18; Gross A. et al., Curr Opin Chem Eng. 2013 Feb. 1; 2(1):3-7; Swiers G et al. Nat Commun. 2013; 4:2924; Bonnet D et al., Bioconjug Chem. 2006 November-December; 17(6):1618-23, and WO 2013155222 A2, all of which are incorporated by reference in their entirety.

In other embodiments, the neural precursors of the invention can be differentiated from a pluripotent stem cell or neural stem cell population. Specific pluripotent stem cells and various methods of neural differentiation that may be useful for differentiation are disclosed, for example, in U.S. Pat. Apps. 20150004701, 20140335059, 20140308745, 20140113372, 20130004985, 20120328579, 20120322146, 2011031883, 20110070205, 20110002897, 20100291042, 20100287638, 20090263361, 20090220466, 20080254004, 20070231302, 20070020608, 20060270034, 20060211111, 20060078545, 20060008451, and 20050095702, all of which are incorporated by reference in their entirety.

In some embodiments, the neural precursor cell populations are created through reprogramming of cells, e.g., neural cells obtained from the MGE, Cortex, Sub-Cortex, other regions of the brain, or non-neural cells. Methods for reprogramming that may be useful in the present invention are disclosed, e.g., U.S. Pat App. 20150087594, 20150086649, 20130109090, and 20130109089; See also Takahashi, K., et al. Cell 131, 861-872 (2007) and U.S. App. No. 20130022583.

In some embodiments, the neural precursor cell populations are created through direct reprogramming of non-neural cells, e.g., pluripotent stem cells, fibroblasts, blood cells, or non-neuronal glial cells (Colasante G et al., Cell Stem Cell, 2015, 17, 719-34; Shi Z, et al., Journal of Biological Chemistry, 2016, 291(26), 13560-70; Sun A et al., Cell Reports, 2016, 16, 1942-53)

### Therapeutic Administration Methods

Methods of administering the neural precursor cells of the invention of the present disclosure to animals, particularly humans, are described in detail herein, and include injection or implantation of the neural precursor cells of the invention into target sites in the subject. The cells of the disclosure can be inserted into a delivery device which facilitates introduction by, injection or implantation, of the cells into the animals. Such delivery devices include tubes, e.g., catheters, for injecting cells and fluids into the body of a recipient animal. In a preferred embodiment, the tubes additionally have a needle, e.g., a syringe, through which the cells can be introduced into the animal at a desired location. The neural precursor cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable carrier or diluent in which the cells remain viable. Pharmaceutically acceptable carriers and diluents include saline, aqueous buffer solutions, solvents and/or dispersion media. The use of such carriers and diluents is well known in the art. The solution is preferably sterile and fluid to facilitate delivery. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the present disclosure can be prepared as described herein in as a pharmaceutically acceptable carrier or diluent and, as required, other ingredients enumerated above, followed by filter sterilization.

In humans, injections will generally be made with sterilized 10 μl Hamilton syringes having 23-27 gauge needles. The syringe, loaded with cells, is mounted directly into the head of a stereotaxic frame. The injection needle is lowered to predetermined coordinates through small burr holes in the cranium, 40-50 μl of suspension are deposited at the rate of about 1-2 μl/minute and a further 2-5 minutes are allowed for diffusion prior to slow retraction of the needle. Frequently, two or more separate deposits will be made, separated by 1-3 mm, along the same needle penetration, and up to 5 deposits scattered over the target area can readily be made in the same operation. The injection may be performed manually or by an infusion pump. At the completion of surgery following retraction of the needle, the patient is removed from the frame and the wound is sutured. Prophylactic antibiotics or immunosuppressive therapy may be administered as needed.

### Therapeutic Indications Amenable to Treatment

In some embodiments, the present disclosure is useful in the treatment of degenerative diseases. A degenerative disease is a disease in which the decline (e.g., function, structure, biochemistry) of particular cell type, e.g., neuronal, results in an adverse clinical condition. For example, Parkinson's disease is a degenerative disease in the central nervous system, e.g., basal ganglia, which is characterized by rhythmical muscular tremors, rigidity of movement, festination, droopy posture and masklike facies. Degenerative diseases that can be treated with the substantially homogenous cell populations of the present disclosure include, for example, Parkinson's disease, multiple sclerosis, epilepsy, Huntington's, dystonia, (dystonia musculmusculorum deformans) and choreoathetosis.

In some embodiments, the present disclosure is useful in the treatment of conditions caused by an acute injury. An acute injury condition is a condition in which an event or multiple events results in an adverse clinical condition. The event which results in the acute injury condition can be an external event such as blunt force or compression (e.g., certain forms of traumatic brain injury) or an internal physiological event such as sudden ischemia (e.g., stroke or heart attack). Acute injury conditions that can be treated with the cell populations of the present invention include, but are not limited to, spinal cord injury, traumatic brain injury, brain damage resulting from myocardial infarction and stroke.

In some embodiments, the administered cells comprise a substantially homogenous population of cells, which may be obtained from isolation from a primary source or from derivation of the cells from a pluripotent or multipotent stem cell source. In some embodiments, the substantially homogenous population comprises cells wherein at least 25% of the cells become GABA expressing cells. In some embodiments, the substantially homogenous population comprises cells wherein at least 30%, 40%, 50%, 60%, 70%, 80%, 90%, 95%, or 99% of the cells become GABA expressing inhibitory interneurons. In some embodiments, at least 25% of the cells comprising the substantially homogenous population of cells migrate at least 0.5 mm from the injection site. In some embodiments, at least 30%, 40%, 50%, 60%, 70%, 80%, 90%, 95%, or 99% of the cells comprising the substantially homogenous population of cells migrate at least 0.5 mm from the injection site. In some embodiments, the majority of the cells comprising the substantially homogenous population of cells migrate at least 1.0, 1.5, 2.0, 3.0, 4.0, or 5.0 mm from the injection site. In some embodiments, at least 25% of the substantially homogenous population of cells becomes functionally GABAergic interneurons. In some embodiments, at least 30%, 40%, 50%, 60%, 70%, 80%, 90%, 95%, or 99% of the cells become functionally GABAergic interneurons. In some embodiments, at least 25% of the substantially homogenous population of cells becomes functionally GABAergic interneurons that integrate with endogenous neurons. In some embodiments, at least 30%, 40%, 50%, 60%, 70%, 80%, 90%, 95%, or 99% of the substantially homogenous population of cells become functionally GABAergic interneurons that integrate with endogenous neurons.

Selected cells can be used directly from cultures or stored for future use. e.g., by cryopreserving in liquid nitrogen. Other methods of cryopreservation are also known in the art, e.g., U.S. Pat. App. 20080057040. If cyropreserved, neural precursor cells of the invention must be initially thawed before placing the neural precursor cells of the invention in a transplantation medium. Methods of freezing and thawing cryopreserved materials such that they are active after the thawing process are well-known to those of ordinary skill in the art.

In some embodiments, the present disclosure includes a pharmaceutical composition comprising a substantially homogeneous cell population of neural precursor cells. In some embodiments, the pharmaceutical composition has at least about 10̂3 or 10⁵ substantially homogeneous cells. In some embodiments, the pharmaceutical composition has at least about 10⁶, 10⁷, 10⁸, 10⁹, or 10¹⁰ substantially homogeneous cells. The cells comprising the pharmaceutical composition can also express at least one neurotransmitter, neurotrophic factor, inhibitory factor, or cytokine.

The neural precursor cell populations of the present invention can be, for example, transplanted or placed in the central, e.g., brain or spinal cord, or peripheral nervous system. The site of placement in the nervous system for the cells of the present disclosure is determined based on the particular neurological condition, e.g., direct injection into the lesioned striatum, spinal cord parenchyma, or dorsal ganglia. For example, cells of the present disclosure can be placed in or near the striatum of patients suffering from Parkinson's disease. Similarly, cells of the present disclosure can be placed in or near the spinal cord (e.g., cervical, thoracic, lumbar or sacral) of patients suffering from a spinal cord injury. One skilled in the art would be able to determine the manner (e.g., needle injection or placement, more invasive surgery) most suitable for placement of the cells depending upon the location of the neurological condition and the medical condition of the patient.

The neural precursor cell populations of the present invention can be administered alone or as admixtures with conventional excipients, for example, pharmaceutically, or physiologically, acceptable organic, or inorganic carrier substances suitable for enteral or parenteral application which do not deleteriously react with the cells of the present disclosure. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the cells of the present disclosure.

When parenteral application is needed or desired, particularly suitable admixtures for the cells are injectable, sterile solutions, preferably oily or aqueous solutions, as well as suspensions, emulsions, or implants. In particular, carriers for parenteral administration include aqueous solutions of dextrose, saline, pure water, ethanol, glycerol, propylene glycol, peanut oil, sesame oil and polyoxyethylene-block polymers. Pharmaceutical admixtures suitable for use in the present disclosure are well-known to those of skill in the art and are described, for example, in Pharmaceutical Sciences (17th Ed., Mack Pub. Co., Easton, Pa.) and WO 96/05309 the teachings of both of which are hereby incorporated by reference.

The neural precursor cell populations can be used alone or in combination with other therapies when administered to a human suffering from a neurological condition. For example, steroids or pharmaceutical synthetic drugs can be co-administered with the cells of the present disclosure. Likewise, treatment of spinal cord injury can include the administration/transplantation of the cells of the present disclosure in a human whose spine has been physically stabilized.

The dosage and frequency (single or multiple doses) of the administration or transplantation of the cells to a human, including the actual number of cells transplanted into the human, can vary depending upon a variety of factors, including the particular condition being treated, e.g., degenerative condition, acute injury, neurological condition; size; age; sex; health; body weight; body mass index; diet; nature and extent of symptoms of the neurological condition being treated, e.g., early onset Parkinson's disease versus advanced Parkinson's disease; spinal cord trauma versus partial or complete severing of the spinal cord); kind of concurrent treatment, e.g., steroids; complications from the neurological condition; extent of tolerance to the treatment or other health-related problems. Humans with a degenerative condition, acute injury, or neurological condition can be treated of once or repeatedly with cells of the present disclosure, e.g., about 10⁶ cells, at the same or different site. Treatment can be performed monthly, every six months, yearly, biannually, every 5, 10, or 15 years, or any other appropriate time period as deemed medically necessary.

The methods of the present disclosure can be employed to treat neurological conditions in mammals other than human mammals. For example, a non-human mammal in need of veterinary treatment, e.g., companion animals (e.g., dogs, cats), farm animals (e.g., cows, sheep, pigs, horses) and laboratory animals (e.g., rats, mice, guinea pigs).

### EXAMPLES

The following examples are put forth so as to provide those of ordinary skill in the art with a complete disclosure and description of how to make and use the present invention, and are not intended to limit the scope of what the inventors regard as their invention, nor are the examples intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1: Cell Enrichment of Neural Precursors of Interest from Human Cortex

Mouse inhibitory interneuron precursor transplants have been shown to be efficacious in the brain and spinal cord of multiple preclinical models including epilepsy, Parkinson's, autism, Alzheimer's disease, and neuropathic pain (U.S. Pat. App. 20090311222, U.S. Pat. App. 20130202568). Global gene expression profiling of the developing human fetal brain was examined using RNA sequencing to identify novel transcript expression in human interneurons and in precursors of human interneurons to identify cells with the ability to migrate and differentiate into inhibitory interneurons in vivo. These markers examined comprised both intracellular markers and markers expressed on the cell surface.

In a specific example, three cell surface markers were utilized to enrich for neural precursors from fetal human tissue. Human fetal brain tissue was placed in cold HibE (Thermo Fisher, Carlsbad, Calif.) and dissected under a stereological microscope using autoclave-sterilized surgical tools. Dissected tissue (1-2 cm²) was placed into a new plate containing cold HBSS (Thermo Fisher, Carlsbad, Calif.).

Dissected brain tissue was further dissociated by placing the brain tissue in cold HBSS buffer and cutting it into small pieces. Cut tissue was washed with cold PBS twice, and incubated with pre-warmed (4 ml) TrypLE (Thermo Fisher, Carlsbad, Calif.) at 37° C. for 10 minutes. The reaction was quenched using a large volume (25-40 ml) of 100 μg/ml DNAse (Roche Molecular Systems, Pleasanton, Calif.) and 140 μg/ml ovomucoid (Worthington, Lakewood, N.J.) in HBSS. Cells were then dissociated from the digested tissue mechanically using a 10 ml pipet and the mixture was passed through a 40 um cell strainer. The cell suspension was centrifuged at 300×g for 5 min and the resultant cell pellet washed twice in cold HBSS. Cells were then resuspended in cold HBSS with 1% BSA, 0.1% glucose (FACS buffer) and counted with Trypan blue. Other forms of tissue dissociation, e.g. using dispase, accutase, papain or other enzymatic and/or mechanical methods, can also be used.

Tissue debulking was achieved using methods such as centrifugation using other gradients and/or magnetic bead-based separation. Tissue was debulked in the present experiment using approximately twenty million human dissociated cortical cells in 4 ml of cold FACS buffer were carefully layered on top of 8 ml of cold 10% Percoll (Sigma, St. Louis, Mo.) and centrifuged at 500×g for 20 minutes. The pellet was then washed twice with 10 ml cold HBSS and cells resuspended in cold FACS buffer.

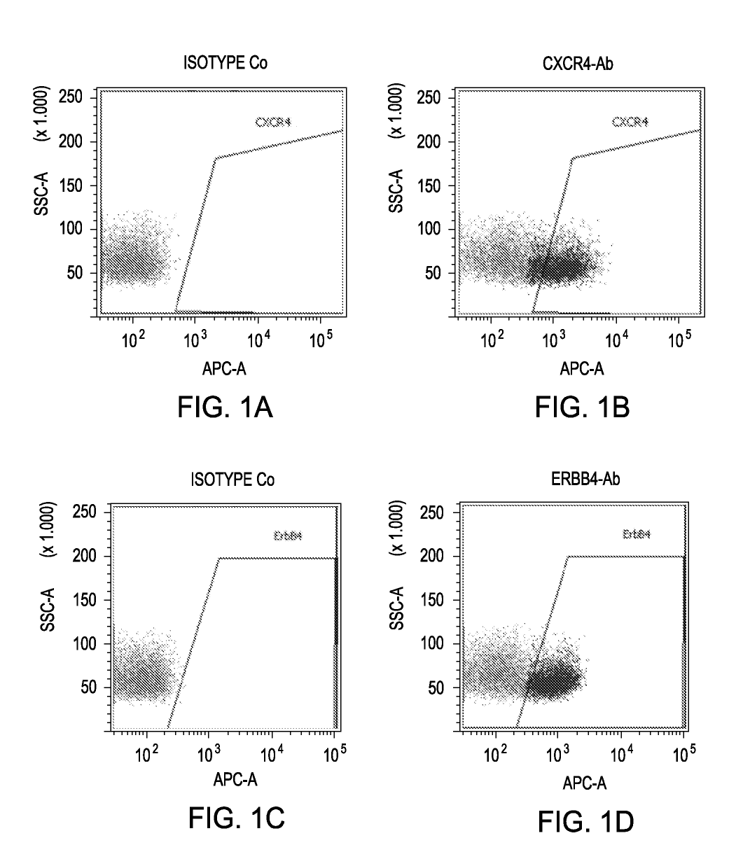

Three neural cell surface markers expressed by MGE-derived cortical interneurons, CXCR4, CXCR7 and ERBB4, were used for the enrichment of cell populations using antibody-based purification of the cells from the fetal brain, including the use of APC-conjugated anti-CXCR4 antibodies (FIGS. 1A and 1B) and APC-conjugated anti-ErbB4 antibodies (FIGS. 1C and 1D). Unstained cells and isotype control antibodies were used as gating controls.

Approximately five million human dissociated cortical cells were resuspended in 250 μl of FACS buffer and incubated with Human BD Fc Block™ (BD Pharmigen, 1:50 dilution) for 10 minutes at 4° C. APC-conjugated primary antibodies were then added to the cells at a final dilution of 1:25 and incubated for 30-40 min at 4° C. After two washes with cold FACS buffer, cells were resuspended in 500 μl of FACS buffer with 5 uM Sytox Blue (Thermo Fisher, Carlsbad, Calif.), collected in a 5 ml polystyrene tube with cell-strainer cap (Falcon) and analyzed using a BD FACS-Aria Cell Sorter (Beckton Dickonson, Franklin Lakes, N.J.). SytoxBlue was used to discriminate dead (Sytox positive) from live (Sytox negative) cells. APC positive and negative cell fractions were collected into 15 ml tubes (Corning, Corning N.Y.) containing 5 ml of NS media (Neurobasal A, B27 (supplemented with Vitamin A), Pen/strept and glutamine) Cell fractions were then centrifuged at 500×g for 5 min and resuspended in 300 μl of RLT buffer (Qiagen, Hilden, Germany) containing beta-mercaptoethanol and stored at −80° C. Alternatively, 5000-10000 APC-positive and negative cells were collected in 96-well plates coated with Matrigel (growth factor reduced) and cultured in 150 μl of NS media for 48 hours at 37° C.

In vitro assays such as RT-PCR and immunocytochemistry were used to confirm the identity of the purified cells using expression of markers specific to cells of the MGE lineage. RNA of sorted cells (collected in RLT buffer) was isolated using RNEasy Micro kit (Qiagen, Hilden, Germany) and cDNA was synthesized using SuperScript III reverse transcriptase (ThermoFisher, Carlsbad, Calif.). RT-PCR was carried out using SYBR Green. Primers against LHX6, DLX2 and SOX6 were used to detect MGE interneurons.

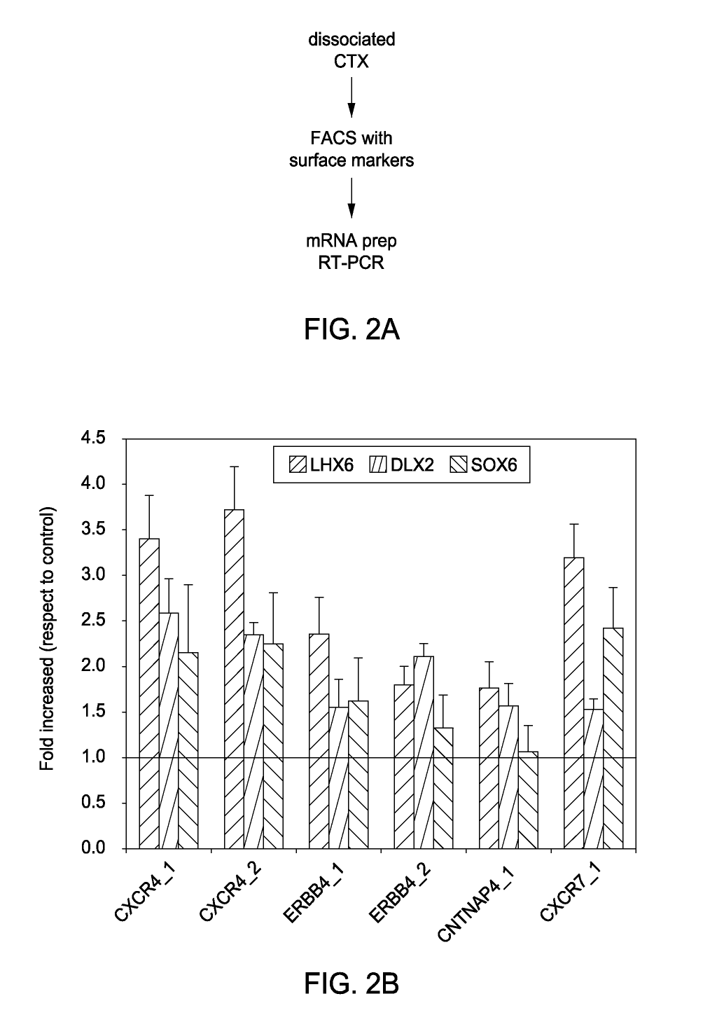

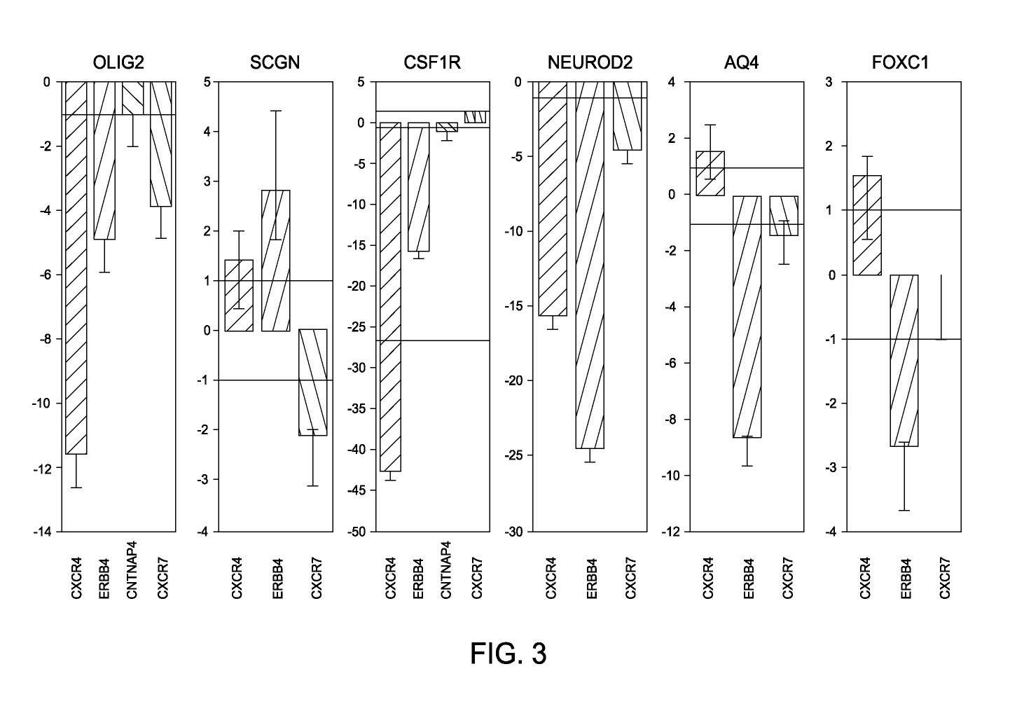

As shown in FIG. 2, the MGE-specific markers LHX6, DLX2 and SOX6 were enriched in the FACS purified cell populations. Primers against OLIG2, SCGN, CSF1R, NEUROD2, AQ4, VAMP1 and FOXC1 were used to detect contaminating populations of oligodendroglia, CGE interneurons, microglia, excitatory cortical neurons, astroglia, pericytes, and endothelial cells, respectively. As shown in FIG. 3, FACS purification mostly selected against contaminating cell populations as the isolated cells had decreased expression of markers of oligodendroglia (OLIG2), CGE interneurons (SCGN), microglia (CSF1R), excitatory neurons (NEUROD2), astroglia (AQ2), and endothelial cells (FOXC1). Exceptions were cells purified using CXCR4, which expressed the SCGN, AQ4 and FOXC1, and cells purified using ERBB4, which express SCGN. The latter cells were excluded from further characterization.

The purified cortical interneuron populations were then validated by immunohistochemistry. After 48 hrs of culture, sorted cells in 96-well plates were fixed in 4% PFA (Affimetrix, Santa Clara, Calif.) for 7 minutes at room temperature and washed with PBS. Wells containing cells were then blocked with Blocking solution (10% donkey serum (Sigma, St Louis, Mo.), 1% BSA (Sigma, St Louis, Mo.), 0.1% Triton X100, 0.1% sodium azide and PBS) for lhr. Fixed cells were incubated with primary antibodies at 4° C. overnight followed by Alexa Fluor fluorescent conjugated secondary antibodies (ThermoFisher, Carlsbad, Calif.) at room temperature for 2 hours. Antibodies used to identify interneurons were: GABA (Sigma, St Louis, Mo.), VGAT (Synaptic Systems, Goettingen, Germany), GAD65/67 (Millipore, Temecula, Calif.), and DLX2. Antibodies against LHX6 (Santa Cruz, Dallas Tex.), MAFB (Sigma, St Louis, Mo.), and CMAF (Santa Cruz, Dallas Tex.) were used to identify MGE-derived interneurons. Other antibodies corresponded to: SP8, DCX, OLIG2 (Millipore, Temecula, Calif.), GFAP (Millipore, Temecula, Calif.), IBA1 and PU1 (Millipore, Temecula, Calif.), KI67, and cleaved-Caspase3 (Millipore, Temecula, Calif.) to detect CGE-derived interneurons, immature neurons, oligodendrocytes, radial glia/astrocytes, microglia, proliferating cells, and apoptotic cells, respectively. Stained cells were analyzed and imaged in a Leica Dmi8 microscope.

The CXCR4+ cells expressed the human nuclear antigen (HNA), the neuroblast marker DCX and the MGE marker LHX6, and the majority expressed the MGE marker MAFB and the vesicular GABA transporter (VGAT). Cells isolated using ERBB4 and CXCR7 antibodies also mostly expressed VGAT. These results showed that the FACS purified cell populations had minimal contamination with proliferating cells and oligodendroglia.

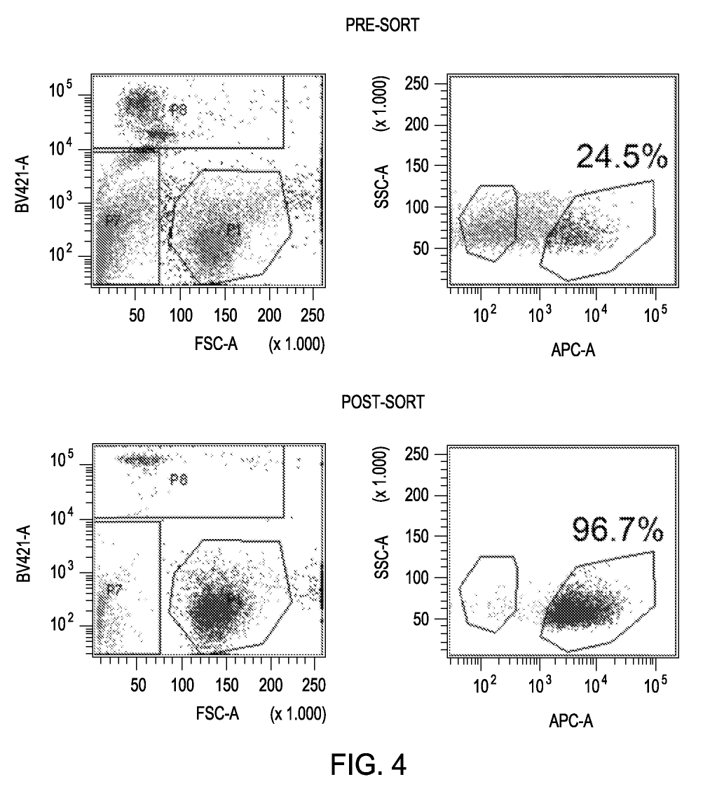

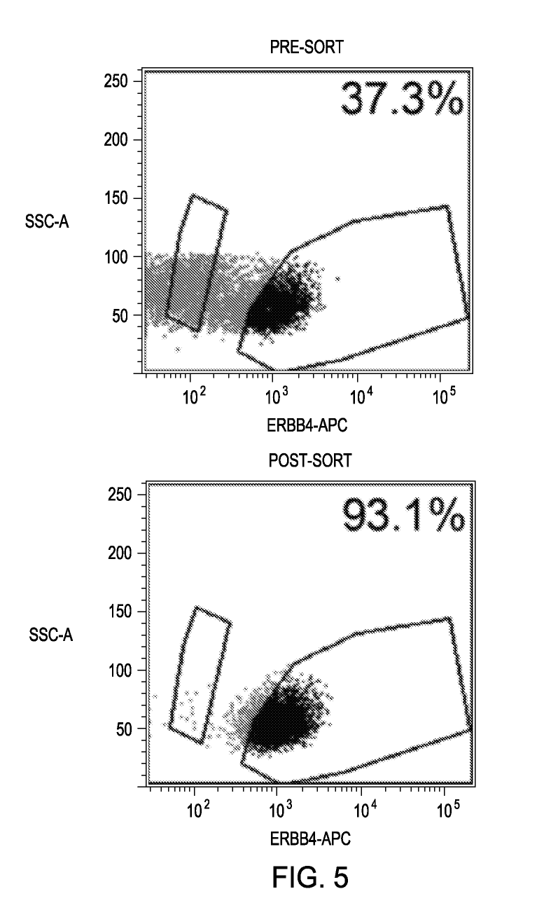

The purified cell populations were shown to have less debris and significantly fewer dead cells than the pre-sorted cell population. The cortical tissue from a gestational week 18 (GW18) brain was dissociated. The dissociated cells were sorted with CXCR4 antibodies and the sorted cells were transplanted into neonatal (P0-P2) mouse pups. As shown in FIG. 4, the pre-sorted cells have a large population of cell debris (P5) and dead (BV421-A+) cells (FIGS. 4A and 4B), but cells sorted using the CXCR4 marker have only a little cell debris (P5) and almost no dead (BV421-A+) cells (FIGS. 4C and 4D). The same was seen for cells sorted using the ERBB4 neural cell surface marker (FIGS. 5A and 5B).

To ensure the cell characteristics were not somehow biased by the enrichment method, cells were then isolated using magnetic-activated cell sorting (MACS). Ten million human dissociated cortical/MGE cells were resuspended in 500 μl of buffer and incubated with Human BD Fc Block™ for 10 minutes at 4° C. Biotinylated primary antibodies were then added to the cells and incubated for 30-40 minutes at 4° C. After two washes with cold buffer, cells were resuspended in FACS buffer containing anti-biotin microbeads and incubated for 30 minutes at 4° C. After two washes with buffer, cells were resuspended in 500 μl of buffer and added to an LS column held on a magnet. The flow-through is collected as “negative sort” and bound material was washed three times. The column was then removed from the magnet and 5 ml of FACS buffer was added and the “positive fraction” was collected. Cell fractions were then analyzed by flow cytometry or immunostaining.

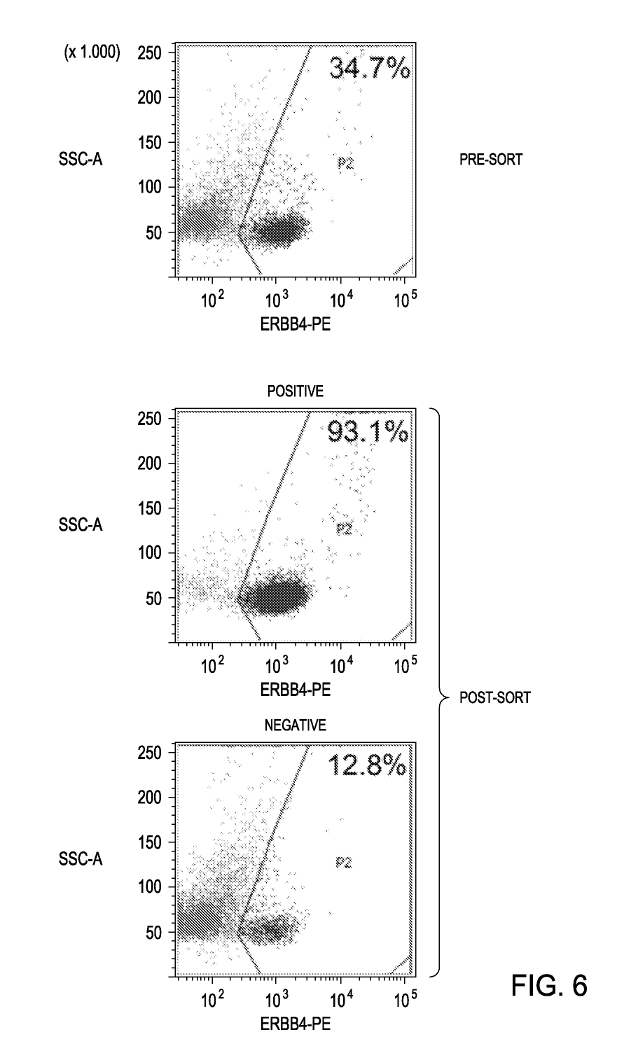

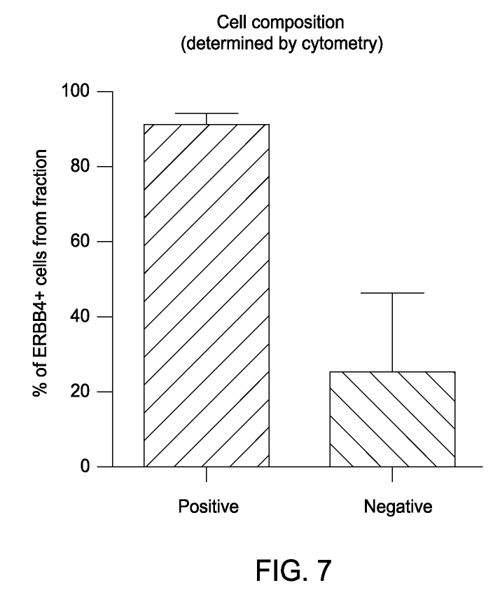

FIG. 6 shows graphs illustrating the efficiency of MACS sorting of human cortical interneurons using magnetic bead-conjugated anti-neural precursor cell-surface antibodies and magnetic column sorting to separate cell surface marker positive and negative populations followed by post-sort flow cytometry analysis to determine the purity of the magnetic separation. ERBB4+ cells from human cortical samples (“pre-sort”) were enriched in the positive magnetic column-bound fraction (“post-sort positive”) while depleted in the flow-through (“post-sort negative”). FIG. 7 is a graph showing MACS separation efficiency summary of the cell surface marker sorting of the cells from human cortical samples (n=7).

The MACS-sorted populations from the human cortical tissue were analyzed by immunocytochemistry (ICC) analysis. After 48 hours of culture, sorted cells in 96-well plates are fixed in 4% PFA (Affimetrix, Santa Clara, Calif.) for 7 minutes at room temperature and washed with PBS. Wells containing cells are then blocked with Blocking solution (10% donkey serum (Sigma), 1% BSA (Sigma), 0.1% Triton X100, 0.1% sodium azide and PBS) for 1 hour. Fixed cells were incubated with primary antibodies at 4° C. overnight followed by secondary antibodies at room temperature for 2 hours. Antibodies used included LHX6 (Santa Cruz), SP8, DCX, OLIG2, GFAP, NEUROD2, SOX10 (Millipore) and ERBB4. Secondary antibodies included AlexaFluor conjugated antibodies (ThermoFisher). Stained cells are analyzed and imaged in a Leica Dmi8 microscope. The total cell number was determined using DAPI staining.

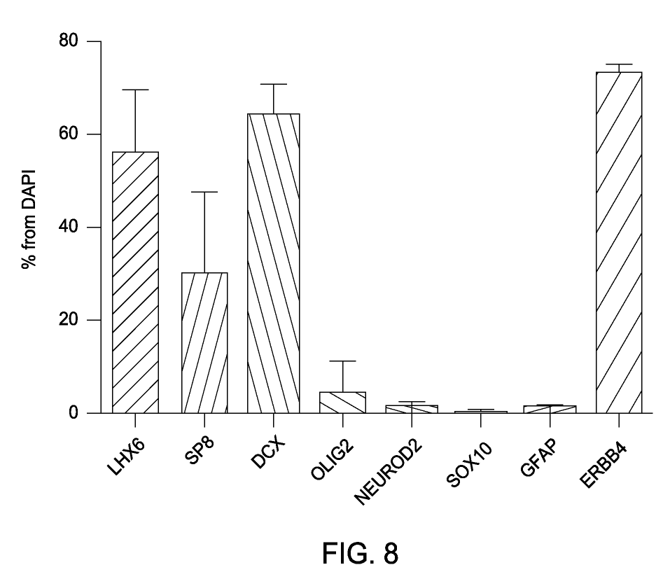

FIG. 8 is a graph showing ICC analysis of the MACS-sorted ERBB4+ population to be enriched for interneuron markers (LHX6, SP8, DCX, ERBB4) and depleted for markers of other cell lineages such as projection neurons (NEUROD2), oligodendrocytes (OLIG2, SOX10), and astrocytes/radial glia (GFAP) (n=4 independent experiments).

### Example 2: Increased Expression of Markers of Neural Precursors of Interest in Cells from Human Cortex

The expression of specific markers of neural precursor cells of interest were examined in cells isolated from human cortex. RNA-sequence analyses of the FACS-sorted populations of interneurons from human cortex prepared as per Example 1 were then performed. mRNA was isolated from each of the three purified cell populations (CXCR4 selected, CXCR7-selected and ERBB4-selected) as well as from the cells in each sample that were not selected using standard techniques mRNA was purified using an RNeasy RNA purification kit (Qiagen, Hilden, Germany), and RNA sequencing was carried out by according to the method described in S. Wang, et al, Plant Cell Rep. (2014) 33(10): 1687-96. Following adapter ligation and PCR amplification the library was then clustered and sequenced.

The mRNA for each group—mRNA from FACS selected and non-selected cell populations—was subject to bulk cell RNA sequencing (Wang et al., Id.) and expression analysis was performed to identify the transcripts with greatest change in expression in the FACS selected cells in comparison to the non-selected cells from the corresponding sample. In brief, samples were sequenced on Illumina Hiseq 2500, low quality reads were trimmed, and remaining high quality reads were mapped to the following reference genome-HomoSapiens Hg19 GRCh37: http://hgdownload.cs.ucsc.edu/downloads.html#human. RPKM values were calculated for each gene and compared between groups.

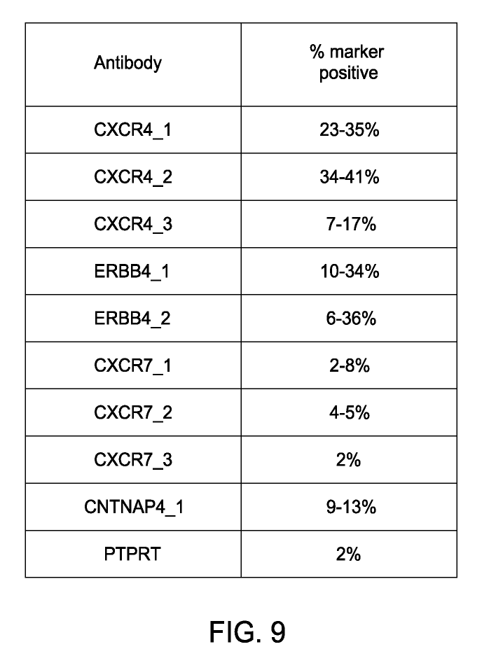

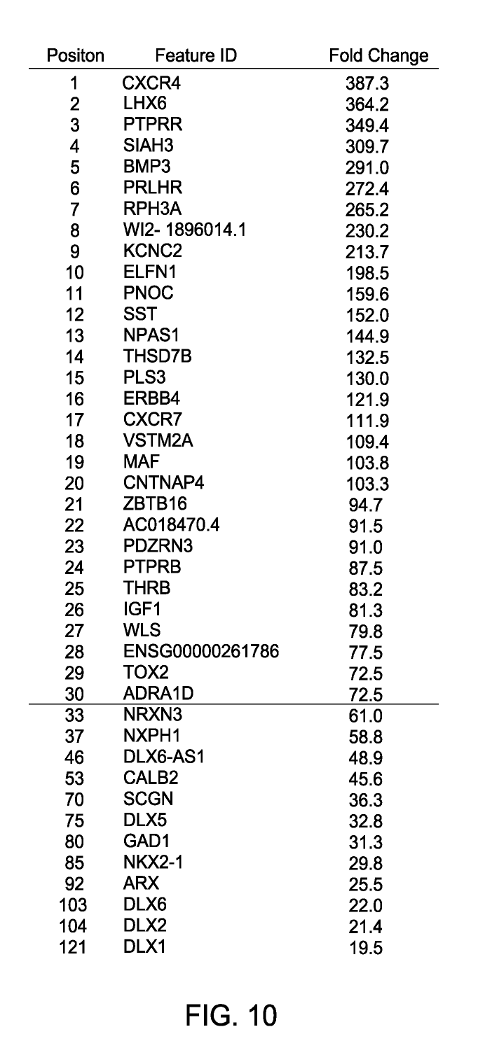

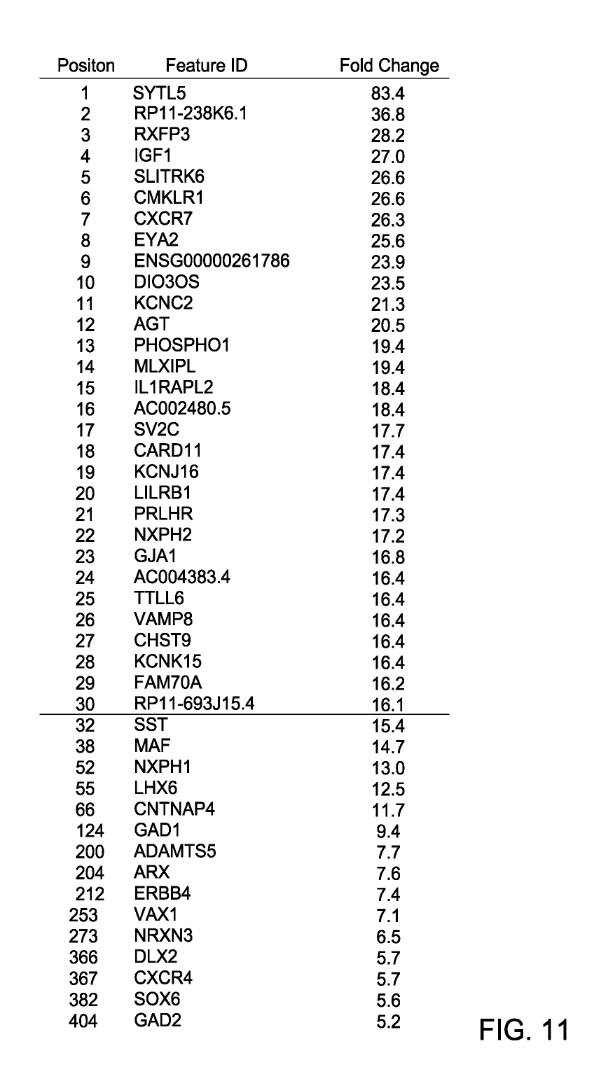

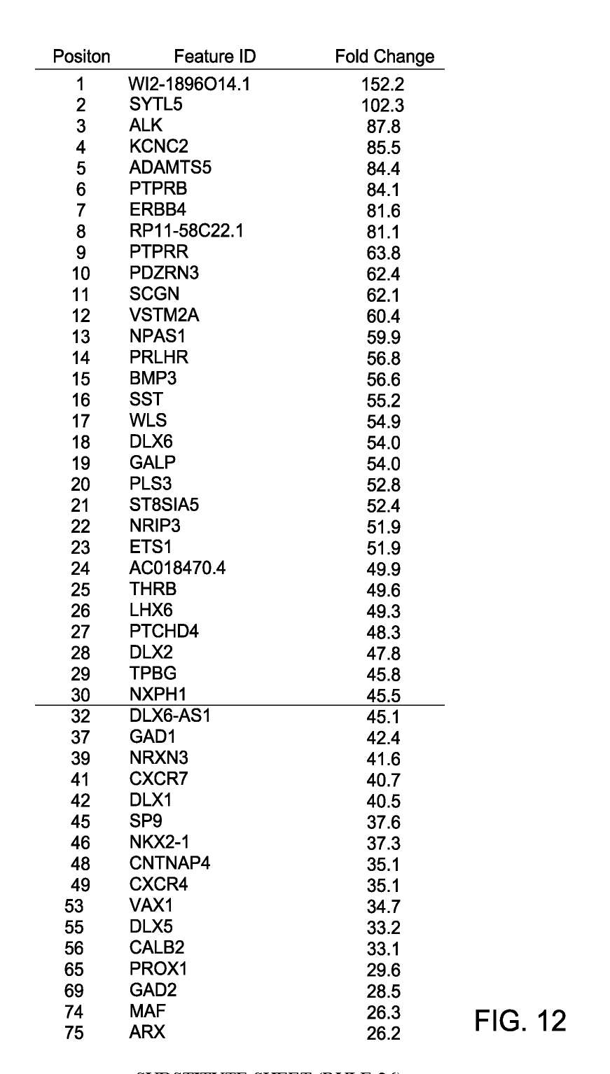

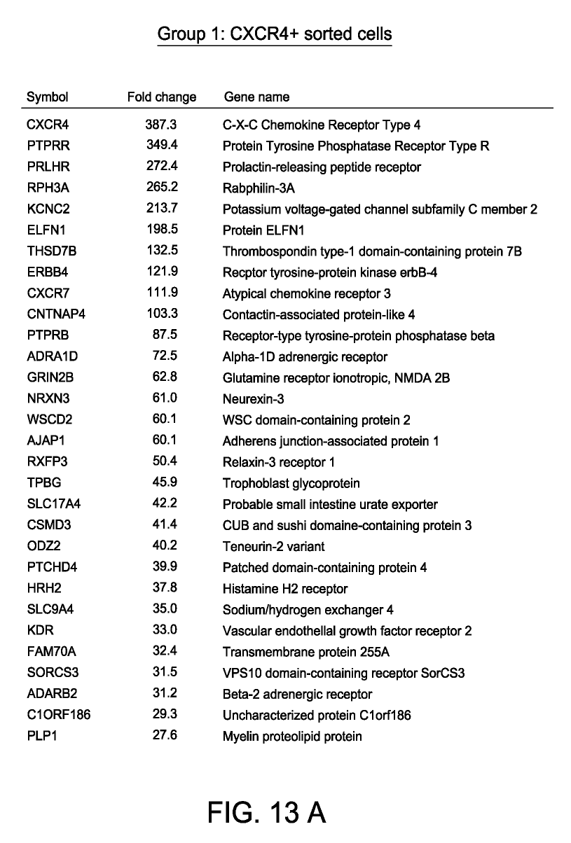

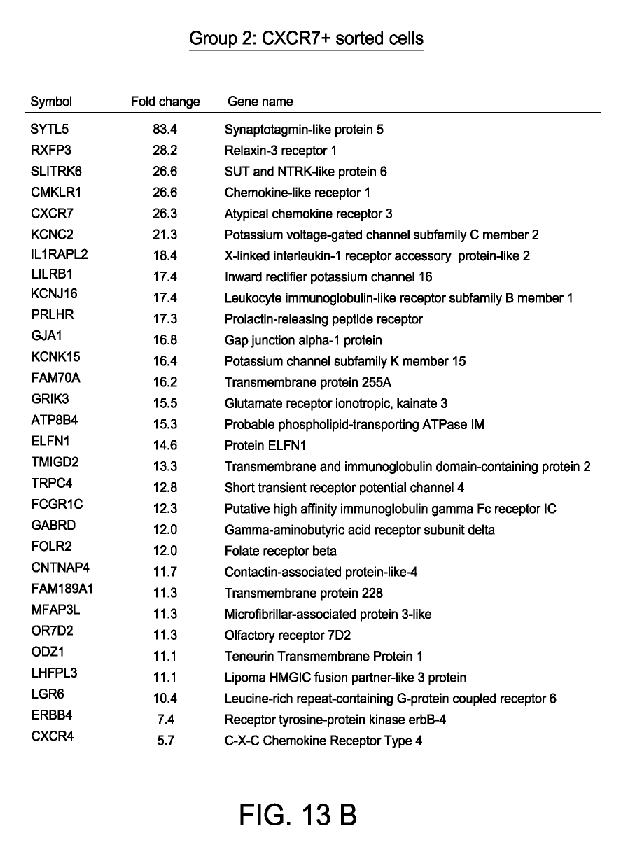

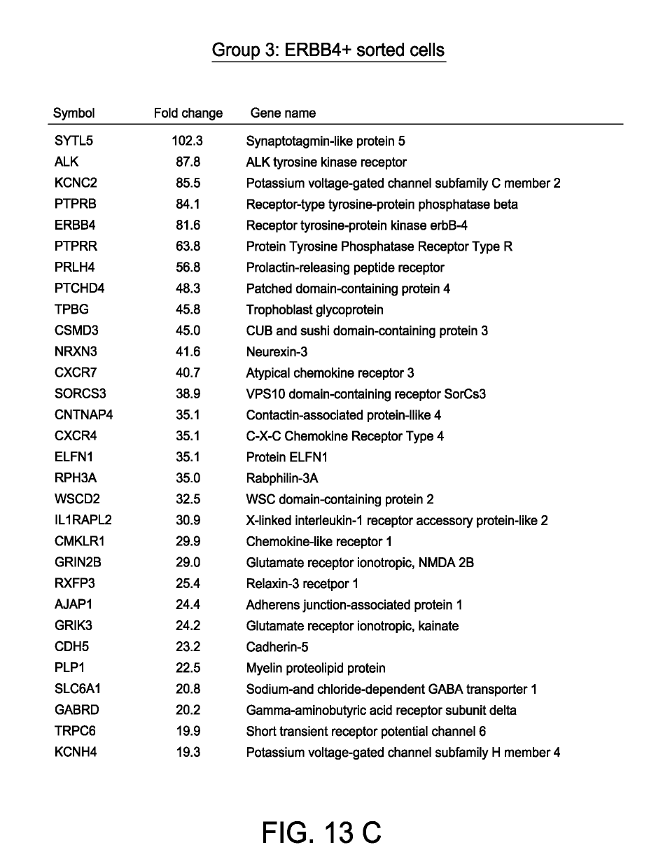

The expression of exemplary cell surface markers is shown in FIG. 9. The thirty most enriched transcripts along with select interneuron marker enriched transcripts are shown in FIGS. 10-12 for each individual cell-surface marker, CXCR4 (FIG. 10), CXCR7 (FIG. 11) and ERBB4 (FIG. 12). The most enriched surface marker transcripts in the CXCR4-selected, CXCR7-selected and ERBB4-selected cell populations are shown in FIGS. 13A, 13B and 13C, respectively.

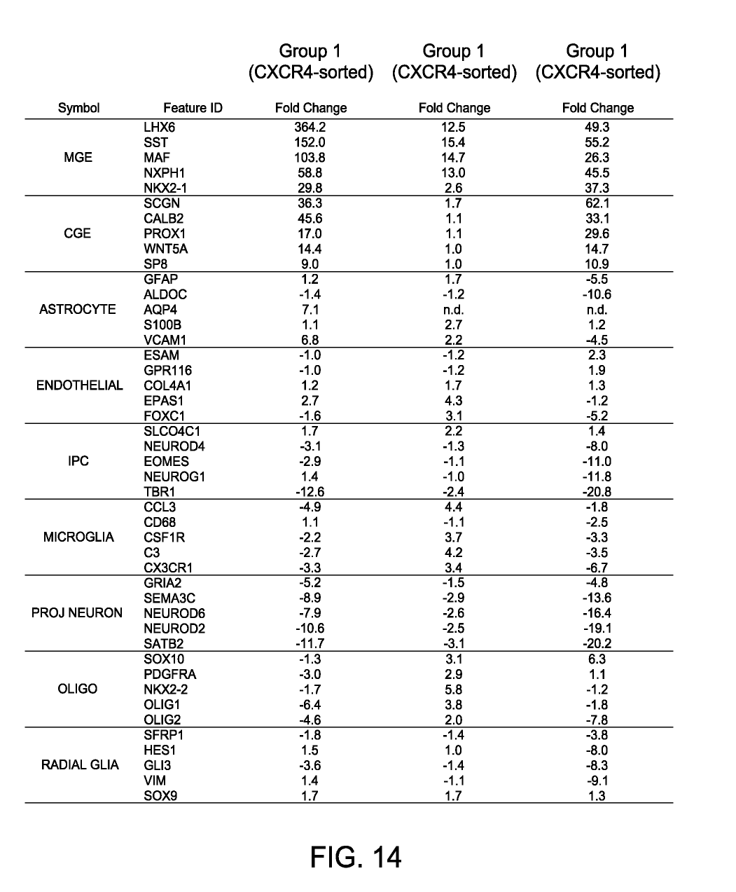

To evaluate the cell-type composition of the sorted populations, a panel of markers of various cell lineages is shown with transcript fold changes in the NPCSM positive populations, over their respective negative populations. MGE- and CGE-type interneuron marker transcripts are enriched in the NPCSM+ populations, whereas transcripts marking non-interneuron cell lineages are largely depleted (FIG. 14).

### Example 3: GABA Expression in Selected Neural Precursors

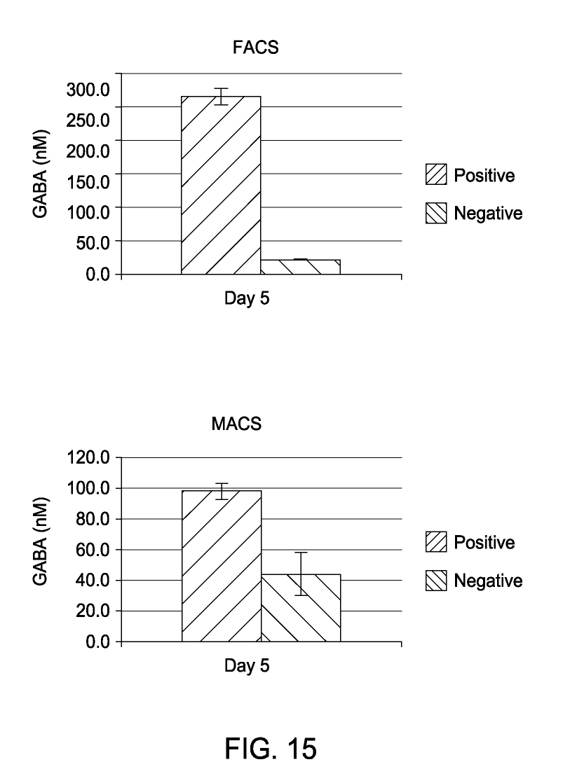

The neural precursor cells prepared from human cortical tissue expressing cell surface markers (e.g., CRCX4, CRCX7, or ERBB4) were shown to express and secrete GABA in vitro following enrichment by either FACS sorting or MACS sorting and culturing. Five days post sorting the cells from the human cortical tissue, the neural precursor cell marker positive and neural precursor cell marker negative cell cultures were analyzed for GABA secretion by HPLC analysis. FIG. 15 shows the increased GABA secretion in the cultured neural precursor cell marker positive populations from human cortical samples using FACS (left panel) or MACS (right panel).

### Example 4: Antero-Posterior Migration and Fate of Cell Surface Marker Positive Cells from Human Cortex Transplanted into Mouse Brain

To determine the ability of the neural precursor cell marker positive cells enriched from human cortical tissue to migrate and differentiate into interneurons in vivo, the neural precursor cell marker positive cells sorted by FACS or MACS were concentrated and transplanted into the neonatal mouse cortex. The concentrated cell suspension was loaded into a beveled glass micropipette (Wiretrol 5 μl, Drummond Scientific Company) mounted on a hydraulic injector. P0-P2 neonatal SCID pups were anesthetized through hypothermia and positioned in a clay head mold on the injection platform. Using a stereotax, predetermined numbers of cells per injection site were injected transcranially into the cerebral cortex of each pup at 1.0 mm from the midline (sagittal sinus), 2.6 mm from the lambda and 0.3 mm deep from the skin surface. The cells were allowed to migrate and differentiate in vivo in the animals prior to immunohistochemical analysis.

The migration and differentiation of the human neural precursor cells in the rodent brains were identified using staining with antibodies against a human-specific marker, HNA, and concurrent staining with antibodies to known markers of interneurons. Briefly, following the incubation period the mice were sacrificed, and brain tissue was fixed with 4% PFA at 4° C. for 48 hours and washed with PBS. Tissue blocks were sectioned on the cryostat and stored in −80° C. until use. Cryosections were incubated with primary antibodies at 4° C. overnight followed by secondary antibodies at room temperature for 2 hours. Antibodies against DCX and GABA were used to detect interneurons. Antibodies against LHX6, CMAF and MAFB were used to detect MGE-type cortical interneurons and antibodies against COUP-TFII and SP8 were used to detect LGE/CGE-type interneurons.

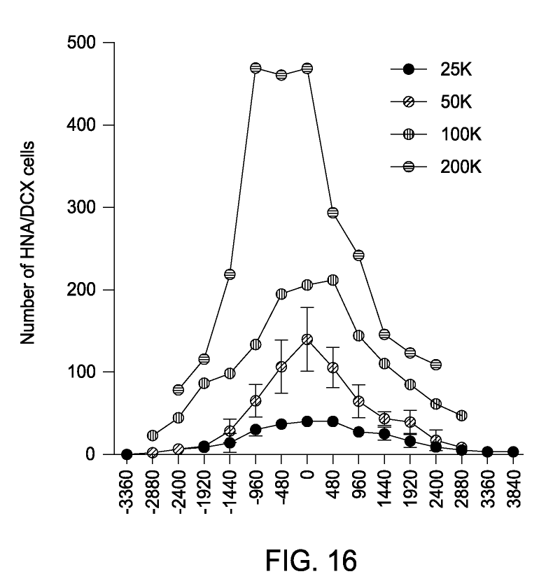

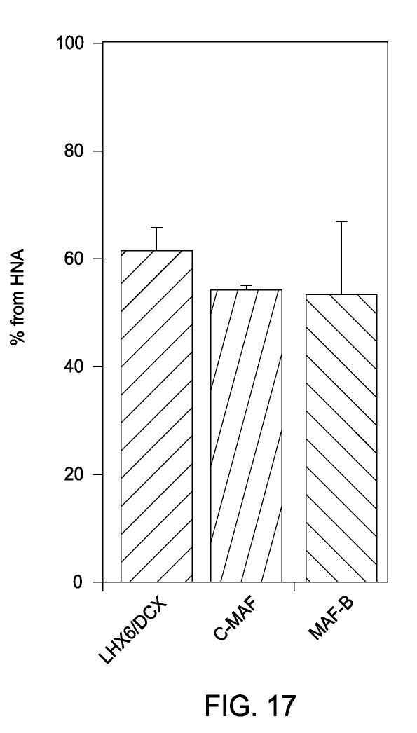

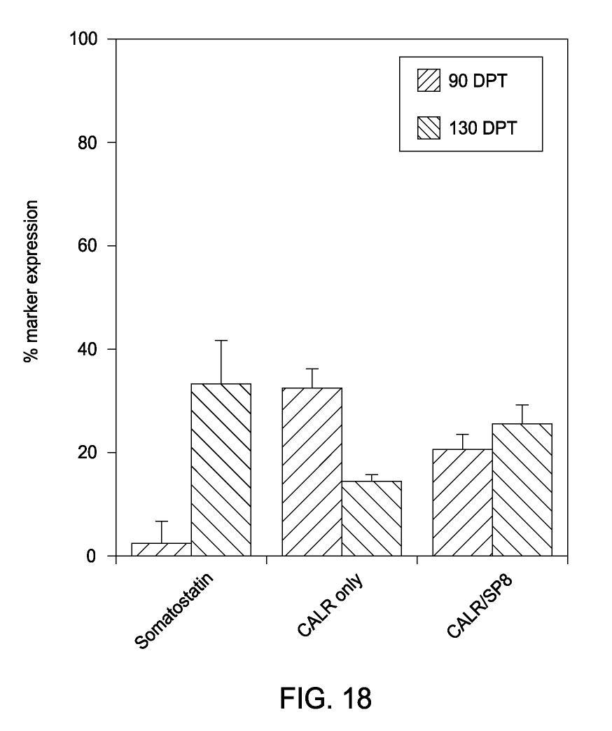

Antero-posterior migration of HNA+/DCX+ cells from their injection site into neonatal mouse cortex, characteristic of migratory interneurons, is shown in FIG. 16. The staining was performed post-injection to identify cell surface marker positive cells sorted from human cortex grafted at different doses (25, 50, 100 and 200×10³ cells per deposit) into the mouse cortex. Human HNA+ cells persisted in the mouse brain 30 days post-transplant (DPT) and also expressed interneuron markers C-MAF, MAF-B, LHX6, and GABA. At 90DPT the cells were still expressing GABA. Quantification of HNA+ cells expressing interneuron markers LHX6, C-MAF, and MAF-B at 30 DPT in the mouse cortex is shown in FIG. 17. Quantification of HNA+ cells expressing more mature interneuron subtype markers SST and CALR (with or without SP8) at 90 DPT and 130 DPT is shown in FIG. 18.

### Example 5: Transplantation of Sorted Human Cortical Cells into Adult Rat CNS

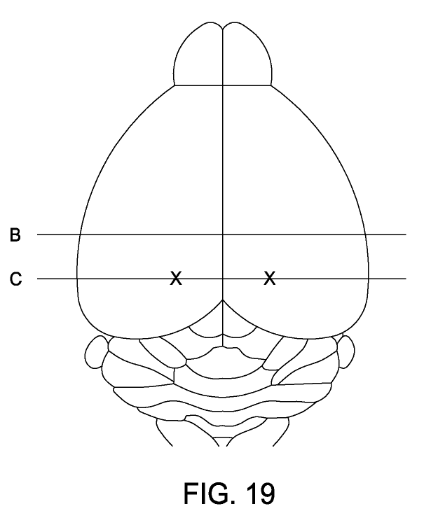

To determine the ability of the neural precursor cell marker positive cells enriched from human cortical tissue to migrate and differentiate into interneurons in vivo in the adult brain, the neural precursor cell marker positive cells sorted by FACS or MACS were concentrated and transplanted into the hippocampus of adult rats. Cell populations were initially sorted by antibodies to either CXCR4 or ERBB4. The concentrated cell suspension was loaded into a beveled glass micropipette (Wiretrol 5 μl, Drummond Scientific Company) mounted on a hydraulic injector. Adult RNU rats were anesthetized through hypothermia and positioned in a clay head mold on the injection platform. The injection sites are illustrated schematically in FIG. 19.

Using a stereotax, a predetermined number of cells per injection site were injected into the adult naïve rat hippocampus. The cells were allowed to migrate and differentiate in vivo in the rats prior to immunohistochemical analysis. Coronal sections were taken at 71 DPT, and stained using antibodies to HNA and interneuron markers MAFB, LH6X and GABA. Cells positive for HNA and interneuron markers were found dispersed within the hippocampus, and the cells displayed migratory interneuron morphology.

Next, the ability of the neural precursor cells sorted from human cortex to migrate and differentiate in an adult diseased mammalian CNS was examined using both the kainate-induced rat epilepsy model and rats with spinal cord contusion injuries. The sorted, concentrated cells were transplanted into the kainate-induced epileptic adult rat hippocampus or injured spinal cord, and the cells allowed to migrate in vivo as described above. Following 71 days, the CNS sections receiving the transplanted cells contained human HNA+DCX+ double positive cells that dispersed in the hippocampus or spinal cord, and these cells both co-expressed the interneuron marker LHX6 and displayed a migratory phenotype.

### Example 6: PLEXINA4 Cell Enrichment and Increased Expression of Markers of Neural Precursors of Interest in Cells from Human Ganglionic Eminences and Human ESC-Derived Cultures

Human ganglionic eminences (medial, caudal, and lateral) at 20 gestational weeks were found to comprise cell populations that express both a neural precursor cell surface marker (“NPCSM”)(e.g., CRCX4, CRCX7 or ERBB4) and PLEXINA4 (Hoch R V et al., Cell Rep. 2015, Jul. 21, 12:3 484-492). Proportionately more PLEXINA4 single positive cells and some NPCSM single positive cells are observed for the medial GE. A similar expression pattern was detected when staining hESC-derived cultures were differentiated towards the MGE lineage.

As described herein for cells from human cortex, cells were isolated from human fetal MGE using an NPCSM (e.g., CRCX4, CRCX7 or ERBB4) to enrich the cells for neural precursor cells of interest, and expression analysis was performed on these enriched cells to identify the transcripts with greatest change in expression in the FACS selected cells in comparison to the non-selected cells from the corresponding sample. In brief, samples were sequenced on Illumina Hiseq 2500, low quality reads were trimmed, and remaining high quality reads were mapped to the following reference genome-HomoSapiens Hg19 GRCh37: http://hgdownload.cse.ucsc.edu/downloads.html#human. RPKM values were calculated for each gene and compared between groups.

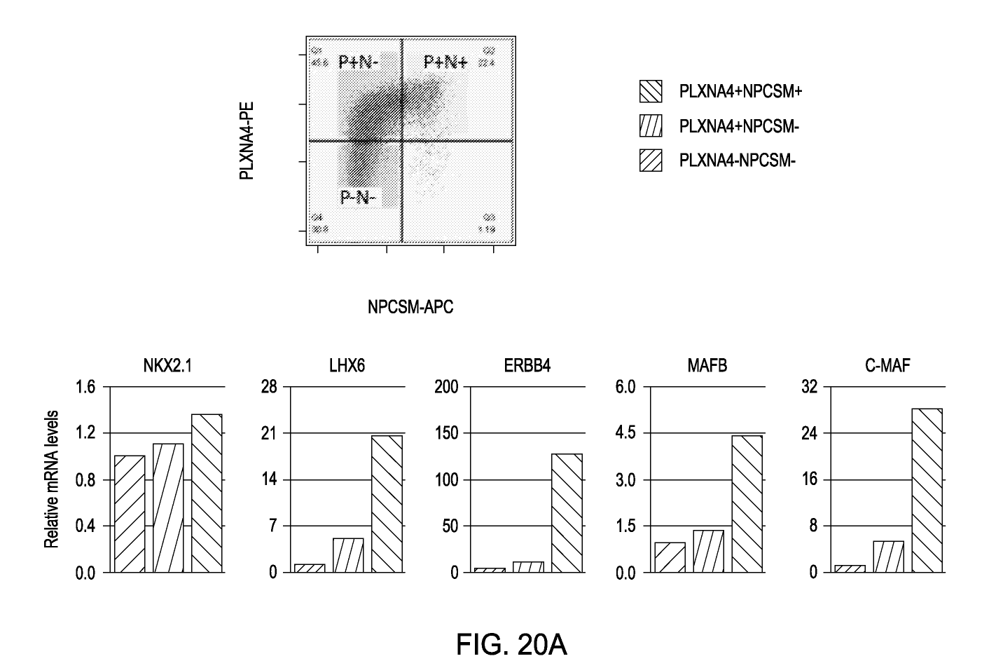

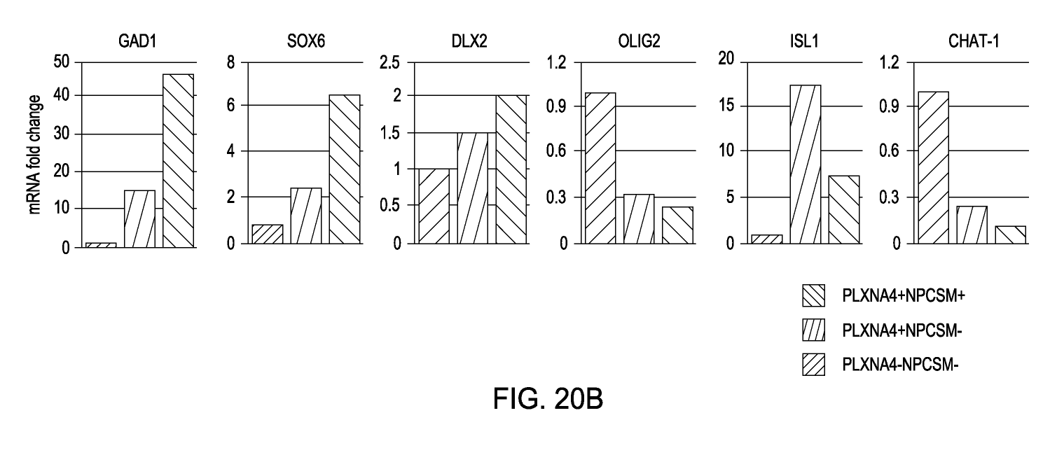

PLXNA4+ NPCSM+ double positive, PLXNA4− NPCSM− double negative, PLXNA4+ NPCSM−, and PLXNA4− NPCSM+ single positive populations were isolated using binding agents. Of note, NPCSM+ binding agents alone may be used to isolate PLXNA4− NPCSM+ and PLXNA4+ NPCSM+ populations. These populations were isolated from human medial GE by FACS sorting using antibodies to the cell-surface markers. The relative gene expression levels in the three cell populations were determined by qRT-PCR (performed as described herein). The NPSCM+ double positive population is enriched for interneuron marker transcripts (LHX6, ERBB4, MAFB, CMAF, GAD1, SOX6, DLX2) (FIGS. 20A and 20B), and depleted for markers of other cell lineages (OLIG2, ISL1, CHAT) relative to total mRNA levels. The PLXNA4 single positive population is also enriched for interneuron marker transcripts, but at lower levels than the PLXNA4+ NPSCM+ population, likely reflecting a more immature stage of development (FIGS. 20A and 20B).

The composition of the three FACS-sorted cell populations from human MGE tissue were then characterized further by immunocytochemistry (ICC) analysis. MGE progenitor markers NKX2.1 and OLIG2 were down-regulated in NPCSM+ cells, and interneuron markers LHX6 and ERBB4 were up-regulated in NPCSM+ cells, with the expression measured as a fold change over expression levels in undifferentiated hES cells. LHX6 was also up-regulated in PLXNA4+ NPSCM− cells, but was not present in detectable levels in the PLXNA4− NPSCM− cells.

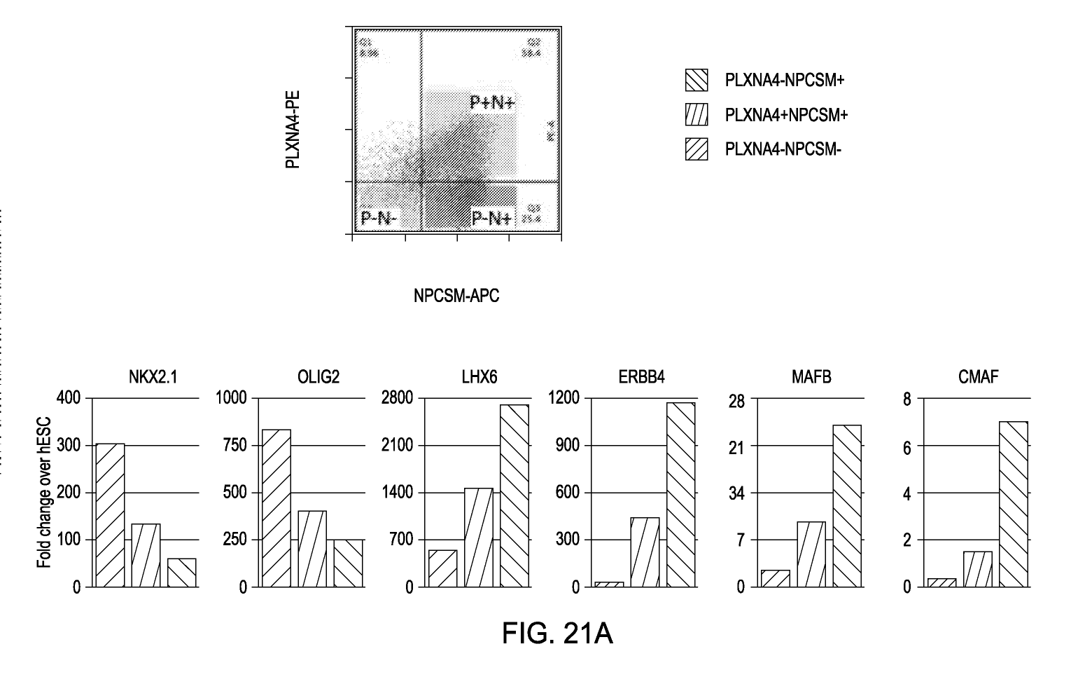

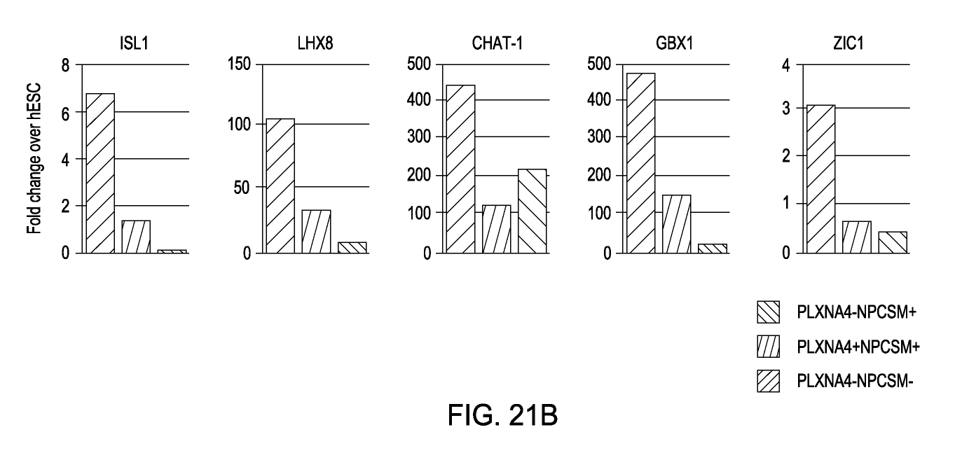

Similarly, double negative, double positive, and NPCSM+ single positive populations were isolated from human ESC-derived MGE patterned cultures by FACS sorting using antibodies to the NPCSM. The relative gene expression levels in the three cell populations were determined by qRT-PCR as described herein. The NPSCM+ single positive population is enriched for interneuron marker transcripts (LHX6, ERBB4, MAFB, CMAF) (FIG. 21A), and depleted for markers of other cell lineages (OLIG2, ISL1, CHAT, LHX8, GBX1 and ZIC1) relative to total mRNA levels. The PLXNA4+ NPCSM+ double positive population is also enriched for interneuron marker transcripts as above, but at lower levels than the NPSCM+ single positive population, likely reflecting a more immature stage of development (FIGS. 21A and 21B).

Global gene expression analysis comparing PLXNA4− NPCSM−, PLXNA4+ NPCSM−, and PLXNA4+ NPCSM+ FACS purified populations from human MGE was examined by RNA sequencing (RNAseq). The top genes listed were either up or down-regulated in the single or double positive population.

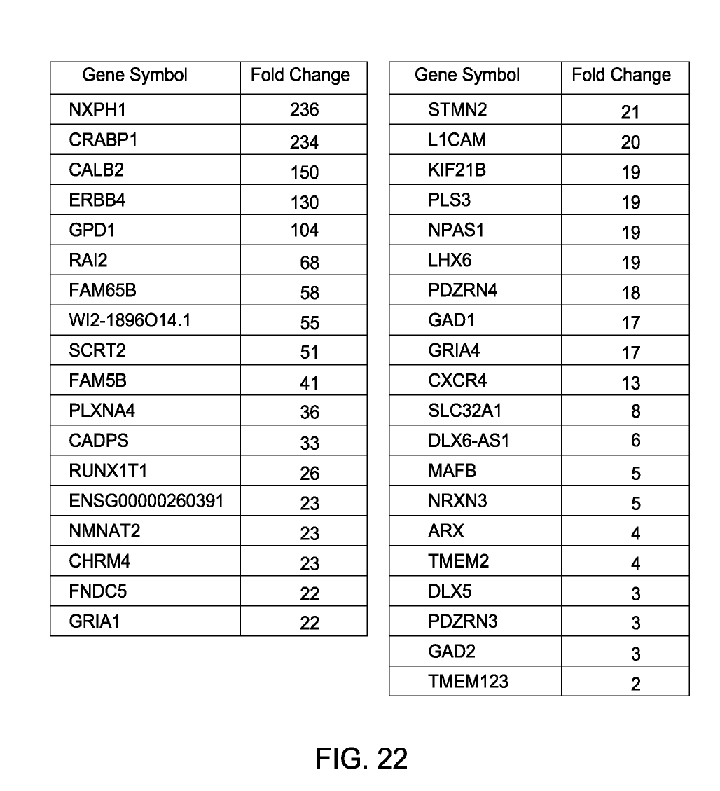

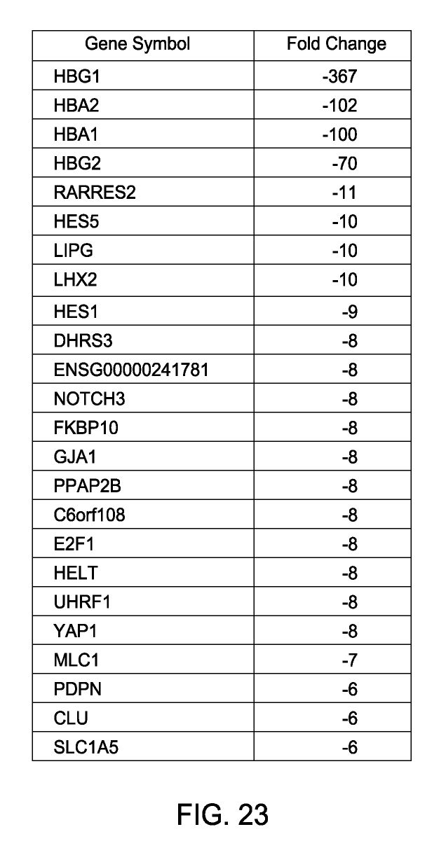

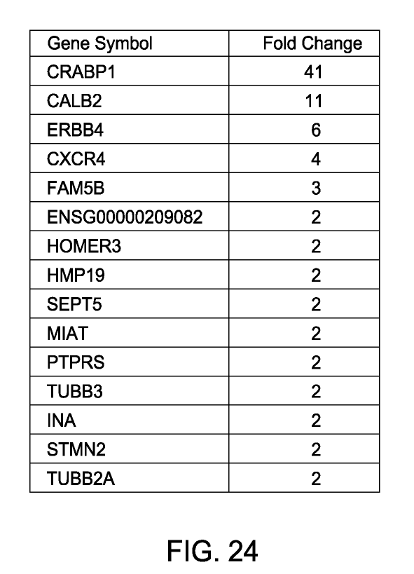

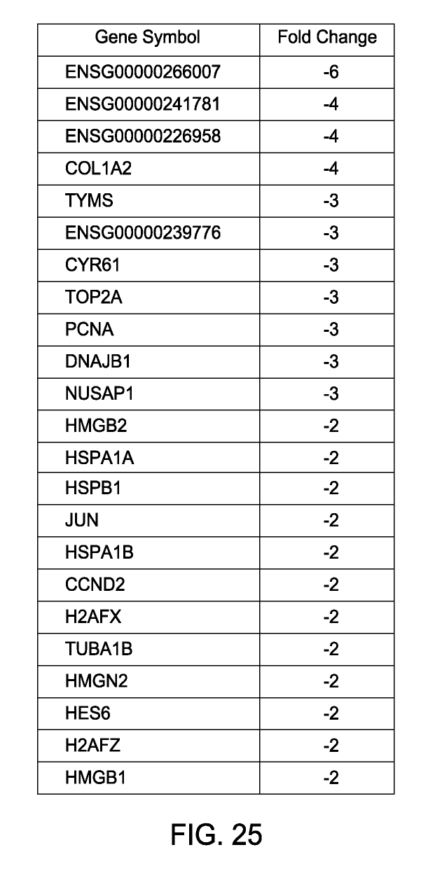

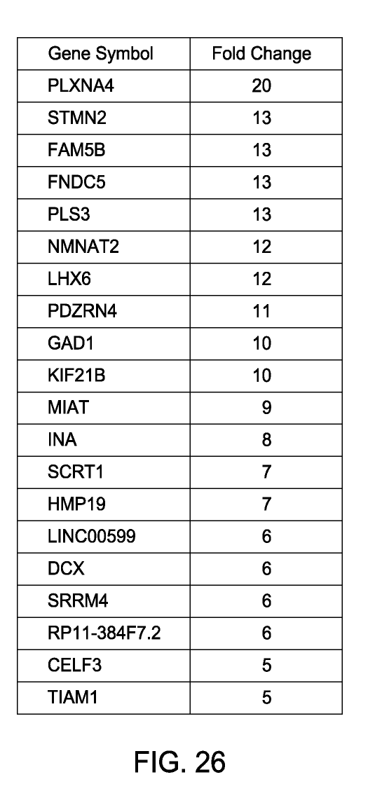

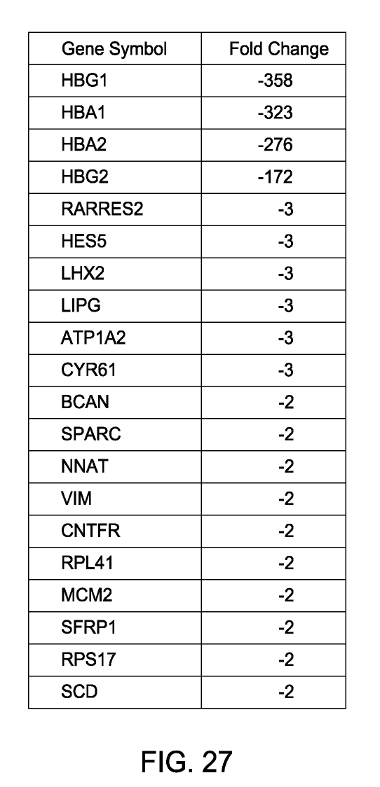

The RNA sequence analysis identified highly-enriched marker transcripts, which are compared by their fold changes in the expression values in comparison to other surface marker sorted cells in each group in Tables 1-3 and FIGS. 22-27. Table 1 shows all differentially expressed transcripts enriched by fold change in the PLEXINA4+ NPCSM+ sorted population as compared to the level of these markers in the PLEXINA4− NPCSM− sorted population. Table 2 shows all differentially expressed transcripts enriched by fold change in the PLEXINA4+ NPCSM+ sorted population as compared to the level of these markers in the PLEXINA4+ NPCSM− sorted population. Table 3 shows all differentially expressed transcripts enriched by fold change in the PLEXINA4+ NPCSM− sorted population as compared to the level of these markers in the PLEXINA4− NPCSM− sorted population. FIG. 22 shows the top 30 enriched neural precursor cell markers, along with additional exemplary interneuron markers, in the PLEXINA4+ NPCSM+ sorted population as compared to the level of these markers in the PLEXINA4− NPCSM− sorted population. FIG. 23 shows the top 20 depleted markers, along with exemplary surface markers, in the PLEXINA4+ NPCSM+ sorted population as compared to the level of these markers in the PLEXINA4− NPCSM− sorted population. FIG. 24 shows the increase in expression of the top 16 neural precursor cell markers in the PLEXINA4+ NPCSM+ sorted population as compared to the level of these markers in the PLEXINA4+ NPCSM− sorted population. FIG. 25 shows the decrease in expression of the top 23 markers in the PLEXINA4+ NPCSM+ sorted population as compared to the level of these markers in the PLEXINA4+ NPCSM− sorted population. FIG. 26 shows the increase in expression of the top 20 neural precursor cell markers in the PLEXINA4+ NPCSM− sorted population as compared to the level of these markers in the PLEXINA4− NPCSM− sorted population. FIG. 27 shows the decrease in expression of the top 20 markers in the PLEXINA4+ NPCSM− sorted population as compared to the level of these markers in the PLEXINA4− NPCSM− sorted population.

TABLE 1
Transcripts by fold change in PLEXINA4+NPCSM+ cells versus
PLEXINA4− NPCSM− cells

| | | PLXNA4+ | | | PLXNA4+ |
|---|---|---|---|---|---|
| | Expression | NPSCM+ versus | | Expression | NPSCM+ versus |
| | Fold Change | PLXNA4− | | Fold Change | PLXNA4− |
| Feature ID | (normalized) | NPSCM− | Feature ID | (normalized) | NPSCM− |
| NXPH1 | 236.159824 | 0.0008578 | J01415.25 | 1.19511376 | 0.00014443 |
| CRABP1 | 233.677195 | 0 | MT-ND4 | 1.19271962 | 0.00185152 |
| CALB2 | 150.37612 | 5.9876E−09 | EEF1A1 | −1.1667786 | 0.01656307 |
| ERBB4 | 130.389113 | 2.2351E−06 | RPS15 | −1.3200419 | 0.00014443 |
| GPD1 | 104.228728 | 0.00372044 | TMSB4X | −1.3441133 | 4.4073E−07 |
| RAI2 | 67.7910877 | 0.00961514 | FOS | −1.3859778 | 0.04320867 |
| FAM65B | 58.3699768 | 0.00028515 | RPS18 | −1.4105604 | 0.04674259 |
| WI2-1896O14.1 | 54.863058 | 4.2501E−05 | RPL10A | −1.4905913 | 0.00194162 |
| SCRT2 | 50.7575738 | 0.0421257 | UBB | −1.5246808 | 2.8764E−08 |
| FAM5B | 41.3459251 | 1.9567E−10 | HNRNPA2B1 | −1.5313866 | 0.00631229 |
| PLXNA4 | 35.8952619 | 2.1455E−13 | HNRNPL | −1.5458617 | 0.01365074 |
| CADPS | 32.666651 | 0.03434241 | HSP90AA1 | −1.5756224 | 0.00181946 |
| RUNX1T1 | 26.2097563 | 0.00111805 | HSP90AB1 | −1.6040142 | 9.2385E−06 |
| ENSG00000260391 | 23.2353859 | 4.1712E−06 | PTMA | −1.6557128 | 4.2977E−09 |
| NMNAT2 | 23.0350272 | 2.0283E−05 | RPS2 | −1.6726281 | 0.00053026 |
| CHRM4 | 22.8921637 | 0.00033509 | HNRNPA3 | −1.6767716 | 0.00733477 |
| FNDC5 | 22.4910731 | 0 | HNRNPAB | −1.6841452 | 0.00286764 |
| GRIA1 | 22.13786 | 9.9685E−07 | RHOB | −1.7511628 | 0.02029367 |
| STMN2 | 20.6776515 | 0 | SRSF2 | −1.7732232 | 0.00678903 |
| L1CAM | 20.3752053 | 0.00014614 | JUND | −1.7801386 | 0.00352527 |
| KIF21B | 19.3698405 | 0 | NCL | −1.7886025 | 0.04473711 |
| PLS3 | 18.8502863 | 0 | SRSF1 | −1.7909044 | 0.03120568 |
| NPAS1 | 18.7396043 | 0.00273295 | FBL | −1.7998317 | 0.00045033 |
| LHX6 | 18.6430976 | 0 | GAPDH | −1.8650587 | 5.6369E−12 |
| PDZRN4 | 17.589206 | 6.0132E−06 | IER2 | −1.9105579 | 0.01109763 |
| GAD1 | 16.8532408 | 0 | CNBP | −1.9178299 | 0.00020259 |
| GRIA4 | 16.7867777 | 0.01259093 | TUBB4B | −1.9972845 | 0.00016688 |
| SCRT1 | 15.4659152 | 1.5576E−08 | LMNB2 | −2.0107459 | 0.03310969 |
| MIAT | 15.2172124 | 0 | NUCKS1 | −2.025609 | 0.04559316 |
| HMP19 | 13.7506684 | 0 | PRDX1 | −2.0690445 | 0.01903338 |
| KALRN | 13.4881535 | 0.00364275 | LDHB | −2.08117 | 0.00010984 |
| CXCR4 | 13.4044901 | 1.5139E−07 | RPLP1 | −2.1229809 | 2.6449E−06 |
| TTC9B | 13.155611 | 0.00036532 | NAP1L1 | −2.1341654 | 0.03605238 |
| INA | 13.0703564 | 0 | CALR | −2.1437218 | 0.00011193 |
| NRCAM | 12.8153748 | 4.8844E−05 | RPLP0 | −2.1485278 | 0 |
| LBH | 12.6065233 | 0.00776388 | NPM1 | −2.1576344 | 0.00223216 |
| RP4-791M13.3 | 11.542121 | 0.01036888 | XRCC6 | −2.1645275 | 0.00681633 |
| MAPT | 11.4984116 | 0.00410013 | BANF1 | −2.2137419 | 0.02743409 |
| HIP1R | 11.0016971 | 3.9221E−05 | HSPD1 | −2.2301619 | 0.01420865 |
| CSDC2 | 10.4462537 | 0.00321237 | HMGA1 | −2.2325964 | 0.00684887 |
| OLFM2 | 10.2578259 | 2.5796E−07 | NME2 | −2.240021 | 0.03635565 |
| PDE4DIP | 10.1714358 | 1.9439E−05 | HMGB1 | −2.2802282 | 1.622E−09 |
| C17orf28 | 10.1365191 | 0.00811091 | SAE1 | −2.2972591 | 0.02241267 |
| TSPAN13 | 10.0953899 | 0.01714521 | ENSG00000200434 | −2.3062852 | 0.025027 |
| ROBO1 | 9.95518843 | 8.8436E−05 | MCM7 | −2.3136488 | 2.1089E−06 |
| SMPD3 | 9.22626324 | 0.0001672 | RPN2 | −2.3242084 | 0.04616518 |
| NSG1 | 9.16638798 | 1.2388E−06 | RAN | −2.3598749 | 0.01773395 |
| ACTL6B | 9.14793996 | 0.00028515 | TUBA1B | −2.3600079 | 0 |
| RBP1 | 9.07201562 | 2.5933E−05 | ODC1 | −2.3814532 | 0.00411142 |
| CELF3 | 8.87908337 | 1.8814E−11 | HSP90B1 | −2.3862962 | 0.00406178 |
| RP11-384F7.2 | 8.74582634 | 7.0553E−07 | LMNB1 | −2.3874004 | 0.00012348 |
| DCX | 8.52911591 | 0 | GSTP1 | −2.4153113 | 0.00097854 |
| ADAMTS7 | 8.19312034 | 2.2132E−05 | SAMD1 | −2.4176601 | 0.04392253 |
| KCND3 | 8.01057387 | 0.01727666 | HNRNPF | −2.4352101 | 0.00251805 |
| DSCAML1 | 7.99874765 | 0.00589638 | PA2G4 | −2.4562812 | 0.04453141 |
| LINC00340 | 7.95778616 | 0.00036258 | MKI67 | −2.4894559 | 0.04712623 |
| TAGLN3 | 7.86080025 | 0 | SNRPA | −2.5089031 | 0.00873537 |
| LINC00599 | 7.83522491 | 0.00100896 | H2AFZ | −2.5246646 | 2.207E−10 |
| GPR153 | 7.80759934 | 0.00072176 | PPIA | −2.5376626 | 5.4737E−05 |
| SRRM4 | 7.68368322 | 7.2646E−05 | CKB | −2.5554136 | 0 |
| SLC32A1 | 7.56267823 | 0.00024658 | CKS2 | −2.5652941 | 6.4362E−05 |
| RAB3A | 7.55651271 | 0.00136682 | RNASEH2B | −2.5674664 | 0.04025843 |
| NBEA | 7.41292757 | 0.00725236 | EEF1B2 | −2.5816901 | 0.0006412 |
| CDKN1C | 7.29309632 | 0.03362876 | HSPA5 | −2.6652557 | 0.00292541 |
| PFKFB3 | 7.08880212 | 0.00181852 | DEK | −2.6705793 | 4.0003E−05 |
| GNG2 | 6.95271408 | 9.4393E−05 | NES | −2.6707871 | 3.3671E−09 |
| SH3BP5 | 6.83785147 | 0.03646223 | HMGN2 | −2.6973859 | 0 |
| ELAVL2 | 6.82498968 | 0.03799553 | PID1 | −2.7118449 | 0.0454897 |
| SLAIN1 | 6.80947259 | 0 | SNRPB | −2.7133705 | 6.2447E−05 |
| GDAP1L1 | 6.44144162 | 1.2388E−06 | NASP | −2.7357929 | 0.00022023 |
| DLX6-AS1 | 6.4254092 | 0 | MNF1 | −2.7406636 | 0.04073097 |
| CELSR3 | 6.36426935 | 2.0347E−05 | KIF22 | −2.7617993 | 0.0153214 |
| ARL4D | 6.22314074 | 0 | TPX2 | −2.7686526 | 0.00158977 |
| NRXN2 | 6.2050339 | 0.00640282 | ENO1 | −2.8073632 | 5.209E−06 |
| COL9A3 | 6.1900264 | 0.00558831 | HES4 | −2.8098566 | 0.00010441 |
| ATCAY | 6.12678109 | 3.26E−10 | CDK4 | −2.8157595 | 0.00353478 |
| LZTS1 | 5.99221268 | 0.00490136 | PTBP1 | −2.8247875 | 8.0951E−07 |
| HOMER3 | 5.92637799 | 1.0447E−10 | SLC25A5 | −2.8395921 | 2.5642E−09 |
| ZNF536 | 5.90180837 | 0.00024658 | SMC4 | −2.8626356 | 0.00957372 |
| CPLX2 | 5.88297238 | 0.01496819 | PEA15 | −2.8706908 | 1.0919E−13 |
| IFI44 | 5.79276102 | 1.0614E−05 | COL1A2 | −2.8793874 | 0.00281834 |
| TUBB4A | 5.78499899 | 9.2693E−11 | TPI1 | −2.8990862 | 0.00010789 |
| KIAA1211 | 5.46361877 | 8.2948E−06 | RPL41 | −2.9175907 | 0 |
| DCLK2 | 5.37593192 | 0 | ALYREF | −2.9307667 | 0.00047031 |
| CD200 | 5.37001051 | 0.03779571 | SCRN1 | −2.9402121 | 0.0175874 |
| SEMA6C | 5.36886023 | 6.0708E−07 | ANP32B | −2.9456064 | 2.2898E−05 |
| TIAM1 | 5.31966815 | 0.00181946 | DNAJB1 | −2.9930464 | 0 |
| MLLT11 | 5.30615022 | 5.8456E−07 | KIFC1 | −3.0052057 | 0.00206579 |
| NPTXR | 5.3049871 | 0.00091214 | EGR1 | −3.0380526 | 2.3089E−11 |
| LMBR1L | 5.23655296 | 0.00016662 | HSPA1A | −3.0753481 | 0 |
| APC2 | 5.06523589 | 8.2909E−13 | LSM4 | −3.0821464 | 1.5836E−05 |
| NCAN | 5.05407456 | 4.389E−08 | DNMT1 | −3.0904516 | 0.00570046 |
| TUBB3 | 5.01644818 | 0 | USP1 | −3.1503843 | 0.00352527 |
| AFAP1 | 4.95366009 | 0.00206826 | TOP2A | −3.190524 | 4.8554E−07 |
| KDM6B | 4.93075219 | 0.00016824 | KIF11 | −3.2327995 | 0.02826393 |
| ST8SIA5 | 4.77309298 | 5.3554E−05 | ENC1 | −3.2402622 | 0.0002409 |
| MAFB | 4.76790255 | 0.01896382 | NUSAP1 | −3.2415636 | 1.2151E−07 |
| RP11-566K11.2 | 4.75011091 | 0.00136682 | SOX8 | −3.2447498 | 0.00036258 |
| NRXN3 | 4.62220839 | 0.01041404 | DUT | −3.2888602 | 0.00696529 |
| SCG3 | 4.54947706 | 0.01089895 | CNTFR | −3.2957029 | 7.1028E−06 |
| RUNDC3A | 4.49767013 | 0.00012524 | JUN | −3.3027265 | 0 |
| HIST1H2BD | 4.45981547 | 0.01896382 | DDX12P | −3.3549943 | 0.04674259 |
| ARX | 4.44300442 | 0 | HES6 | −3.3736141 | 0 |
| SEPT5 | 4.41321695 | 1.9411E−12 | CNN3 | −3.4076532 | 0.00032796 |
| SOX11 | 4.40569631 | 0 | HMGCS1 | −3.4145374 | 0.00083812 |
| KIF3C | 4.34587074 | 8.9226E−06 | RRM1 | −3.4234857 | 0.00203193 |
| MEG3 | 4.31699504 | 7.958E−06 | PKM | −3.4300317 | 0.0001678 |
| RTN1 | 4.22150553 | 0.03039717 | HSPA1B | −3.4301097 | 0 |
| TMEM2 | 4.18266974 | 0.00131834 | NR2F1 | −3.4318855 | 0.00558815 |
| KLF7 | 4.16610576 | 0.01430822 | GLO1 | −3.4823042 | 0.0007438 |
| TNFRSF25 | 4.05816548 | 0.02988335 | HMGB2 | −3.4854292 | 0 |
| RUSC1 | 4.0571011 | 0.00032316 | HSPB1 | −3.4902954 | 9.2993E−10 |
| TUBB2A | 4.04558678 | 0 | NNAT | −3.5019653 | 0 |
| PPP1R18 | 4.03488059 | 0.0019309 | H2AFX | −3.5472229 | 0 |
| ST8SIA2 | 4.01773012 | 0.00525093 | ATP1B3 | −3.5649541 | 0.0001248 |
| C1orf187 | 3.9959368 | 0.04320867 | CCND2 | −3.5693505 | 2.0752E−11 |
| SP9 | 3.99053813 | 0 | ASCL1 | −3.582115 | 2.4733E−07 |
| BCL11B | 3.9588581 | 0.00317341 | ANP32E | −3.5928802 | 2.1029E−05 |
| CAMSAP3 | 3.95624077 | 0.03345379 | POLD1 | −3.616817 | 0.00024658 |
| NREP | 3.95352225 | 1.9722E−09 | CDO1 | −3.6405206 | 0.00019493 |
| PPP1R14B | 3.80981282 | 1.9411E−12 | RPL13P12 | −3.654356 | 8.9728E−07 |
| BCL11A | 3.76977492 | 0.04223738 | PBK | −3.6547337 | 0.02090722 |
| ADAMTS10 | 3.76726504 | 2.5626E−05 | UNG | −3.6668348 | 0.00091872 |
| GPC2 | 3.71581508 | 6.6435E−07 | UBE2T | −3.7464461 | 0.00859542 |
| CACNB3 | 3.64084935 | 0.03200051 | MAD2L1 | −3.8175192 | 0.00525093 |
| CCDC136 | 3.62122949 | 0.04514986 | NOTCH1 | −3.8544625 | 4.8554E−07 |
| PLXNA3 | 3.5301826 | 0.00018617 | MFGE8 | −3.896575 | 0.03370118 |
| RBFOX2 | 3.51591027 | 0.00363474 | MYCN | −3.9900221 | 0.00055893 |
| LRP1 | 3.50611576 | 0.00328801 | FOXM1 | −4.0084273 | 0.04093621 |
| MAST1 | 3.4377131 | 0.00045193 | FUZ | −4.0773618 | 0.02347636 |
| C11orf95 | 3.43166033 | 1.9199E−05 | CDK6 | −4.085044 | 0.02272494 |
| NCAM1 | 3.42718075 | 5.1029E−07 | STK39 | −4.1226719 | 0.00013266 |
| KIF5A | 3.41310775 | 0.00020259 | FGFR2 | −4.1273361 | 0.01458496 |
| IGDCC3 | 3.40987914 | 0.03605238 | SCD | −4.184796 | 1.0504E−08 |
| CRMP1 | 3.39894602 | 0 | HSPA6 | −4.2470357 | 0.03880071 |
| hsa-mir-3187 | 3.37285313 | 1.9589E−05 | LTBP4 | −4.2746825 | 0.01155047 |
| FLNC | 3.36420153 | 0.03790576 | TPM2 | −4.3192965 | 0.02000188 |
| ENSG00000209082 | 3.35580197 | 1.3617E−08 | ZNF703 | −4.3415555 | 0.00413944 |
| PLEKHG5 | 3.34924978 | 0.02029367 | PAICS | −4.3478861 | 0.00015788 |
| MYT1 | 3.34790449 | 0.01837783 | SHMT2 | −4.4156867 | 0.04409854 |
| MEX3B | 3.31483038 | 1.0614E−05 | LIG1 | −4.4232864 | 3.8863E−06 |
| PPP1R9B | 3.31165976 | 0.00274094 | DHCR24 | −4.4329441 | 0.0250509 |
| DLX5 | 3.29893152 | 6.2357E−08 | C19orf48 | −4.5193953 | 0.00067403 |
| TUBB2B | 3.19226967 | 0 | TMEM106C | −4.549268 | 0.0094365 |
| PDZRN3 | 3.1434123 | 0.01395353 | PHGDH | −4.5493363 | 0.00047189 |
| PCBP4 | 3.08305416 | 0.00020768 | AHCY | −4.5631405 | 0.01714521 |
| TMSB10 | 3.07922147 | 0 | ATAD2 | −4.5728372 | 0.04664485 |
| BRSK1 | 3.03870256 | 0.00067403 | SFRP1 | −4.5746708 | 3.5339E−11 |
| VAT1 | 3.02517677 | 0.0010967 | SLC9A3R1 | −4.5787857 | 0.03130005 |
| ACAP3 | 3.01169634 | 1.1418E−05 | RNASEH2A | −4.5935202 | 3.5706E−09 |
| MICAL1 | 3.0109055 | 8.0447E−05 | CDCA5 | −4.6730989 | 0.03426117 |
| FAM89B | 3.00885314 | 0.00124973 | SOX21-AS1 | −4.692806 | 0.02318272 |
| CYTH2 | 2.97772141 | 0.01430423 | KAT2A | −4.7025917 | 0.0059027 |
| CERK | 2.96867947 | 0.00163122 | ZWINT | −4.7049629 | 1.8896E−05 |
| SH3BGRL3 | 2.96707648 | 0.00286764 | CHAF1A | −4.7229127 | 0.00022539 |
| ARHGEF2 | 2.91115742 | 0.00307691 | LAPTM4B | −4.7697028 | 0.02090722 |
| SACS | 2.90890431 | 5.6681E−05 | WDR34 | −4.9031015 | 3.2622E−06 |
| SOX4 | 2.87300113 | 0 | LGALS1 | −4.9303583 | 0.00719499 |
| CACNG4 | 2.86432897 | 1.2142E−05 | C2orf72 | −4.9706281 | 0.00337623 |
| APLP1 | 2.85953113 | 2.2696E−05 | MLF1IP | −5.051768 | 8.8741E−05 |
| CORO2B | 2.84900812 | 1.6211E−08 | E2F2 | −5.1287627 | 0.01343356 |
| ELAVL3 | 2.81491317 | 4.0407E−07 | GINS2 | −5.1691729 | 0.00354694 |
| KIAA0895L | 2.77277895 | 0.00443369 | FGFR3 | −5.1908847 | 0.02743409 |
| DCHS1 | 2.75863435 | 0.00062592 | COL9A1 | −5.196946 | 0.00490136 |
| PTPRS | 2.74880954 | 8.965E−10 | SALL1 | −5.2962822 | 0.00363065 |
| IGLON5 | 2.74734108 | 0.00434711 | TIMELESS | −5.3025792 | 2.0283E−05 |
| CLIP2 | 2.73474378 | 0.03809987 | PSAT1 | −5.3961546 | 0.02702683 |
| FEZ1 | 2.73266073 | 0.0022869 | CDT1 | −5.404085 | 0.00363065 |
| PHF21B | 2.68167991 | 0.03728927 | SOX21 | −5.4089407 | 0.0004747 |
| ZSWIM5 | 2.68084612 | 0.00181946 | DHFR | −5.4185793 | 9.9954E−05 |
| VASH1 | 2.680662 | 0.01097572 | TMEM158 | −5.4212427 | 0.00061181 |
| FSCN1 | 2.60771546 | 1.9411E−12 | ENSG00000239776 | −5.436973 | 0 |
| GAD2 | 2.59730986 | 0.03345379 | NR2E1 | −5.4550686 | 7.9916E−05 |
| AGRN | 2.59210809 | 0.03572583 | GMNN | −5.4780211 | 0.02004755 |
| UCHL1 | 2.56043924 | 4.9294E−06 | MCM5 | −5.4814952 | 0.00181946 |
| MIDN | 2.55507979 | 7.7071E−05 | MTHFD1 | −5.5168413 | 0.04559316 |
| DBN1 | 2.53161848 | 4.8133E−10 | RAD51AP1 | −5.5577372 | 0.04218982 |
| DYNC1H1 | 2.51504673 | 0.00421924 | OLIG1 | −5.5685828 | 0.00362396 |
| AC005035.1 | 2.50130524 | 0.00114884 | PCNA | −5.6062798 | 0 |
| NPDC1 | 2.49160351 | 0.01109763 | PTPRZ1 | −5.6158747 | 0.00039965 |
| CCDC88A | 2.48423448 | 0.00879517 | SLC1A5 | −5.6249243 | 0.04490625 |
| MAP2 | 2.45887588 | 0.0016431 | CLU | −5.6295517 | 0.04473711 |
| CDK5R1 | 2.44626311 | 0.00078951 | ENSG00000226958 | −5.6502785 | 0 |
| TERF2IP | 2.44286624 | 0.00234803 | TTYH1 | −5.7193421 | 0.00206826 |
| PLXNB1 | 2.44055914 | 0.00258245 | TYMS | −5.7357356 | 1.6441E−10 |
| ANO8 | 2.43374093 | 0.00124081 | CDC45 | −5.7682952 | 0.02208962 |
| SBK1 | 2.42821906 | 0.0016431 | MCM6 | −5.790149 | 0.00027242 |
| FNBP1L | 2.4121596 | 0.00197308 | TK1 | −5.8365714 | 0.03019258 |
| AES | 2.39451505 | 0 | KIAA1161 | −5.846916 | 0.02004755 |
| MLLT4 | 2.38507045 | 0.02869618 | CDK2 | −5.9166536 | 0.00059159 |
| GDI1 | 2.37112283 | 6.1831E−08 | LYPD1 | −5.9684961 | 0.03345379 |
| RCOR2 | 2.34114801 | 0.00133135 | MYBL2 | −5.9873279 | 2.6391E−07 |
| MAP4K4 | 2.33479641 | 0.00131537 | RRM2 | −6.0712428 | 0.00105117 |
| GSTA4 | 2.33335415 | 0.00793176 | VIM | −6.2172815 | 9.224E−10 |
| SNN | 2.3265104 | 0.00080994 | GSX1 | −6.3020113 | 0.00028515 |
| PFN2 | 2.32584109 | 0 | TNC | −6.3103665 | 0.04008053 |
| SPTAN1 | 2.31489049 | 0.00662258 | PDPN | −6.339063 | 0.01896382 |
| B4GALNT4 | 2.29945045 | 7.0553E−07 | ERF | −6.3857787 | 0.04893972 |
| DPYSL3 | 2.27491506 | 0 | LMO1 | −6.4377982 | 9.0579E−06 |
| PI4KAP1 | 2.27247926 | 0.04019095 | ENSG00000266007 | −6.4650335 | 3.6195E−10 |
| KIAA0182 | 2.23262482 | 0.02700628 | ZFP36L1 | −6.5219275 | 0.00053652 |
| MAGED4 | 2.22838007 | 0.01700286 | KIAA0101 | −6.6287663 | 0.0016431 |
| SEPT3 | 2.22002157 | 0.00074626 | MLC1 | −6.6819056 | 0.0005452 |
| YWHAG | 2.19763215 | 0.00070021 | ASF1B | −6.6941752 | 0.01430822 |
| MAP1B | 2.12781601 | 0.00071287 | SFRP2 | −6.7320538 | 2.4549E−05 |
| HN1 | 2.06354684 | 0.00390749 | MCM3 | −6.7367137 | 2.1782E−07 |
| MARCKSL1 | 2.06326533 | 0 | SIX3 | −6.8120876 | 0.01708389 |
| LPAR2 | 2.05187082 | 0.01032285 | OTX2 | −7.1348618 | 0.00696529 |
| BASP1 | 2.04891028 | 1.9411E−12 | MCM4 | −7.1885013 | 4.8554E−07 |
| ZNF532 | 2.04555813 | 0.02413359 | CYR61 | −7.2538665 | 0 |
| TPGS2 | 2.03750564 | 0.00033509 | SPARC | −7.4353292 | 2.2868E−10 |
| RND3 | 2.03631944 | 0.00215265 | IL33 | −7.5179225 | 0.03678043 |
| DPYSL4 | 2.03613384 | 0.03742005 | DTL | −7.5443985 | 0.04486237 |
| UBA1 | 2.021711 | 0.00032294 | MCM2 | −7.6058246 | 8.234E−10 |
| PDZD4 | 2.00762085 | 0.00346879 | TRIM9 | −7.650713 | 0.00912828 |
| DDAH2 | 1.99176592 | 0.00346879 | ATP1A2 | −7.6734581 | 6.4495E−09 |
| TUBA1A | 1.94787274 | 0 | YAP1 | −7.7721811 | 0.02826393 |
| LDB1 | 1.92722668 | 0.01262703 | UHRF1 | −7.8319773 | 7.7496E−08 |
| RPL9P9 | 1.84480137 | 1.9439E−05 | HELT | −7.8844959 | 1.3573E−10 |
| CCNI | 1.80538618 | 0.00208495 | E2F1 | −7.9007245 | 3.7343E−07 |
| PTMS | 1.78417439 | 5.87E−10 | C6orf108 | −7.903389 | 0.02476899 |
| CLIP3 | 1.78081826 | 0.03426583 | PPAP2B | −7.9981681 | 0.00215176 |
| SOX1 | 1.74684264 | 0.00296426 | GJA1 | −7.9994145 | 0.02569022 |
| FTL | 1.73618039 | 9.2656E−09 | FKBP10 | −8.0055637 | 0.00258205 |
| H3F3B | 1.7218425 | 2.1125E−05 | NOTCH3 | −8.1159073 | 0.00206826 |
| MT-ND1 | 1.63814255 | 4.2169E−13 | ENSG00000241781 | −8.2517259 | 0 |
| PAFAH1B3 | 1.63351967 | 0.04158136 | DHRS3 | −8.3188679 | 0.02836358 |
| EIF4G2 | 1.61277524 | 0.03572583 | HES1 | −9.2985838 | 0.03682236 |
| TMEM123 | 1.57586078 | 0.01060555 | LHX2 | −9.6728486 | 1.5844E−08 |
| ENSG00000211459 | 1.44802406 | 0 | LIPG | −9.8622962 | 1.1366E−06 |
| RPS11 | 1.44444368 | 0 | HES5 | −10.069259 | 0 |
| ENSG00000210082 | 1.34411329 | 4.4073E−07 | RARRES2 | −10.56741 | 1.081E−06 |
| MT-ND2 | 1.3245847 | 0.0011944 | HBG2 | −69.835019 | 0 |
| ACTG1 | 1.31286515 | 4.7074E−09 | HBA1 | −100.25569 | 0 |
| ACTB | 1.21721306 | 0.01420865 | HBA2 | −102.3323 | 0 |
| MT-ATP6 | 1.20333605 | 1.2593E−05 | HBG1 | −366.95527 | 0 |

TABLE 2
Transcripts by fold change in PLEXINA4+ NPCSM+
cells versus PLEXINA4+ NPCSM− cells

| | | PLXNA4+ | | | PLXNA4+ |
|---|---|---|---|---|---|
| | | NPSCM+ | | | NPSCM+ |
| | Expression | versus | | Expression | versus |
| | Fold Change | PLXNA4+ | | Fold Change | PLXNA4+ |
| Feature ID | (normalized) | NPSCM− | Feature ID | (normalized) | NPSCM + |
| CRABP1 | 41 | 0 | NNAT | −1 | 1.672E−05 |
| CALB2 | 11 | 8.7068E−06 | UBB | −1 | 1.1769E−05 |
| ERBB4 | 6 | 0.0113877 | HMGB1 | −2 | 0.00872452 |
| CXCR4 | 4 | 0.00729277 | H2AFZ | −2 | 0.0327426 |
| FAM5B | 3 | 0.0315843 | HES6 | −2 | 0.01008648 |
| ENSG00000209082 | 2 | 0.00423716 | HMGN2 | −2 | 1.199E−08 |
| HOMER3 | 2 | 0.04796135 | TUBA1B | −2 | 2.1768E−10 |
| HMP19 | 2 | 3.5094E−05 | H2AFX | −2 | 0.00257416 |
| SEPT5 | 2 | 0.0327426 | CCND2 | −2 | 0.01137091 |
| MIAT | 2 | 0.0024316 | HSPA1B | −2 | 0 |
| PTPRS | 2 | 0.01185222 | JUN | −2 | 2.828E−11 |
| TUBB3 | 2 | 0 | HSPB1 | −2 | 0.01170716 |
| INA | 2 | 0.04491213 | HSPA1A | −2 | 0 |
| STMN2 | 2 | 3.3204E−06 | HMGB2 | −2 | 3.1711E−07 |
| TUBB2A | 2 | 0.00667635 | NUSAP1 | −3 | 0.00453537 |
| MARCKSL1 | 1 | 2.0272E−08 | DNAJB1 | −3 | 2.1768E−10 |
| MT-ND1 | 1 | 2.8859E−05 | PCNA | −3 | 2.8859E−05 |
| TMSB10 | 1 | 0 | TOP2A | −3 | 0.00028103 |
| TUBB2B | 1 | 1.5082E−07 | CYR61 | −3 | 0.01379399 |
| RPL27 | 1 | 0.0022192 | ENSG00000239776 | −3 | 1.3976E−11 |
| ENSG00000211459 | 1 | 0.0001408 | TYMS | −3 | 0.01676095 |
| ENSG00000210082 | 1 | 0.0212265 | COL1A2 | −4 | 2.6902E−05 |
| TUBA1A | 1 | 0.03588214 | ENSG00000226958 | −4 | 0 |
| RPS11 | 1 | 0.01185222 | ENSG00000241781 | −4 | 6.2732E−07 |
| MT-CO1 | −1 | 0.01185222 | ENSG00000266007 | −6 | 2.3536E−07 |

TABLE 3
Transcripts by fold change in PLEXINA4+ NPCSM−
cells versus PLEXINA4−NPCSM− cells

| | | PLXNA4+ | | | PLXNA4+ |
|---|---|---|---|---|---|
| | Expression | NPSCM− versus | | Expression | NPSCM− versus |
| | Fold Change | PLXNA4− | | Fold Change | PLXNA4− |
| Feature ID | (normalized) | NPSCM− | Feature ID | (normalized) | NPSCM− |
| PLXNA4 | 20 | 1.3678E−06 | TMSB10 | 2 | 0 |
| STMN2 | 13 | 0 | FSCN1 | 2 | 8.4096E−05 |
| FAM5B | 13 | 0.02911083 | GDI1 | 2 | 0.00183691 |
| FNDC5 | 13 | 4.1257E−08 | TMEM123 | 2 | 4.2796E−06 |
| PLS3 | 13 | 0 | TPGS2 | 2 | 0.01177523 |
| NMNAT2 | 12 | 0.04579437 | AES | 2 | 5.2121E−06 |
| LHX6 | 12 | 0 | BASP1 | 2 | 2.0124E−06 |
| PDZRN4 | 11 | 0.00772826 | CCNI | 2 | 0.02860038 |
| GAD1 | 10 | 3.2611E−11 | RPL9P9 | 2 | 0.00632282 |
| KIF21B | 10 | 1.1096E−09 | PTMS | 2 | 6.0498E−06 |
| MIAT | 9 | 0 | ACTG1 | 2 | 0 |
| INA | 8 | 0 | MARCKSL1 | 2 | 8.3403E−07 |
| SCRT1 | 7 | 0.01742446 | TUBA1A | 2 | 9.7833E−13 |
| HMP19 | 7 | 3.6293E−11 | FTL | 1 | 0.00434405 |
| LINC00599 | 6 | 0.03692169 | RPS11 | 1 | 0 |
| DCX | 6 | 0 | H1F0 | 1 | 0.00696851 |
| SRRM4 | 6 | 0.01301498 | MT-ATP6 | 1 | 2.5064E−08 |
| RP11- | 6 | 0.00538152 | J01415.25 | 1 | 0.00362271 |
| 384F7.2 | | | | | |
| CELF3 | 5 | 5.9845E−05 | TMSB4X | −1 | 0.00150098 |
| TIAM1 | 5 | 0.00696851 | RPL13A | −1 | 0.01767032 |
| DLX6-AS1 | 5 | 0 | RPS16 | −1 | 0.00922301 |
| AC017053.1 | 5 | 0.02343861 | HSPA1A | −1 | 3.2229E−09 |
| ARL4D | 5 | 1.0964E−11 | HSPA1B | −1 | 0 |
| TAGLN3 | 5 | 1.512E−05 | HMGN2 | −1 | 0.02039134 |
| SLAIN1 | 5 | 1.4758E−08 | GAPDH | −1 | 0.00617727 |
| DCLK2 | 4 | 0 | JUN | −1 | 0.01288499 |
| GDAP1L1 | 4 | 0.01177523 | RPS15 | −1 | 1.0682E−07 |
| ATCAY | 4 | 0.00019902 | RPLP0 | −2 | 0.00023102 |
| MEG3 | 4 | 0.00029363 | ENSG00000226958 | −2 | 0 |
| KIAA1211 | 4 | 0.01742446 | H2AFX | −2 | 0.02199474 |
| TUBB3 | 4 | 0 | RPS2 | −2 | 0.00300057 |
| ST8SIA5 | 3 | 0.04600176 | PEA15 | −2 | 0.00399024 |
| MLLT11 | 3 | 0.02498151 | RPLP1 | −2 | 0.01689031 |
| SP9 | 3 | 9.7833E−13 | NES | −2 | 0.00912983 |
| TUBB4A | 3 | 0.00468121 | ENSG00000239776 | −2 | 1.6649E−06 |
| SOX11 | 3 | 8.7351E−12 | SLC25A5 | −2 | 0.00575038 |
| ARX | 3 | 4.9822E−12 | HES6 | −2 | 2.4758E−07 |
| DLX5 | 3 | 7.0774E−06 | ENSG00000241781 | −2 | 0.00170522 |
| GAD2 | 3 | 0.01271445 | CKB | −2 | 0 |
| ZSWIM5 | 3 | 0.0011025 | ENO1 | −2 | 0.02688284 |
| SH3BGRL3 | 3 | 0.01723477 | PCNA | −2 | 3.4776E−06 |
| NREP | 3 | 0.00150098 | EGR1 | −2 | 5.9339E−05 |
| APC2 | 3 | 0.00729338 | SCD | −2 | 0.01921224 |
| SACS | 3 | 0.00119053 | RPS17 | −2 | 0.04045938 |
| GPC2 | 3 | 0.01465918 | SFRP1 | −2 | 0.00362271 |
| C11orf95 | 3 | 0.02498151 | MCM2 | −2 | 0.04324907 |
| TUBB2A | 3 | 2.1095E−10 | RPL41 | −2 | 9.2497E−10 |
| NCAM1 | 3 | 0.00772826 | CNTFR | −2 | 0.01656708 |
| MICAL1 | 3 | 0.01689031 | VIM | −2 | 0.00639288 |
| MAGED4 | 2 | 0.00717034 | NNAT | −2 | 0 |
| CCDC88A | 2 | 0.03660984 | SPARC | −2 | 0.00688118 |
| SOX4 | 2 | 0 | BCAN | −2 | 0.04129998 |
| CRMP1 | 2 | 1.4915E−07 | CYR61 | −3 | 4.1963E−07 |
| NBPF1 | 2 | 0.0154065 | ATP1A2 | −3 | 0.00717034 |
| MAP1B | 2 | 0.00081749 | LIPG | −3 | 0.02963571 |
| PFN2 | 2 | 0 | LHX2 | −3 | 0.00458105 |
| ELAVL3 | 2 | 0.00772826 | HES5 | −3 | 7.2486E−06 |
| PPP1R14B | 2 | 0.023733 | RARRES2 | −3 | 0.02281259 |
| DPYSL3 | 2 | 4.7035E−12 | HBG2 | −172 | 0 |
| TUBB2B | 2 | 0 | HBA2 | −276 | 0 |
| RND3 | 2 | 0.00508666 | HBA1 | −323 | 0 |
| DBN1 | 2 | 0.00019701 | HBG1 | −358 | 0 |
| CORO2B | 2 | 0.01689031 | | | |

### Example 8: Production of Neural Precursors of Interest from hESC Cultures

Human ES cell (ESC) lines were cultured in TESR-E8 media (Stem Cell Technologies) on a vitronectin substrate (ThermoFisher). Human ESC were differentiated into MGE-type cultures using an optimized cocktail of morphogens added at specific time points to induce MGE-type interneurons (as described in detail: Ser. No. 14/763,397, Nicholas C et al., Cell Stem Cell. 2013, 12(5):573-86). These cells can be further enriched for neural precursors of interest using the cell-sorting techniques utilized for both human cortical cells and human MGE cells, as described in detail in the above examples.

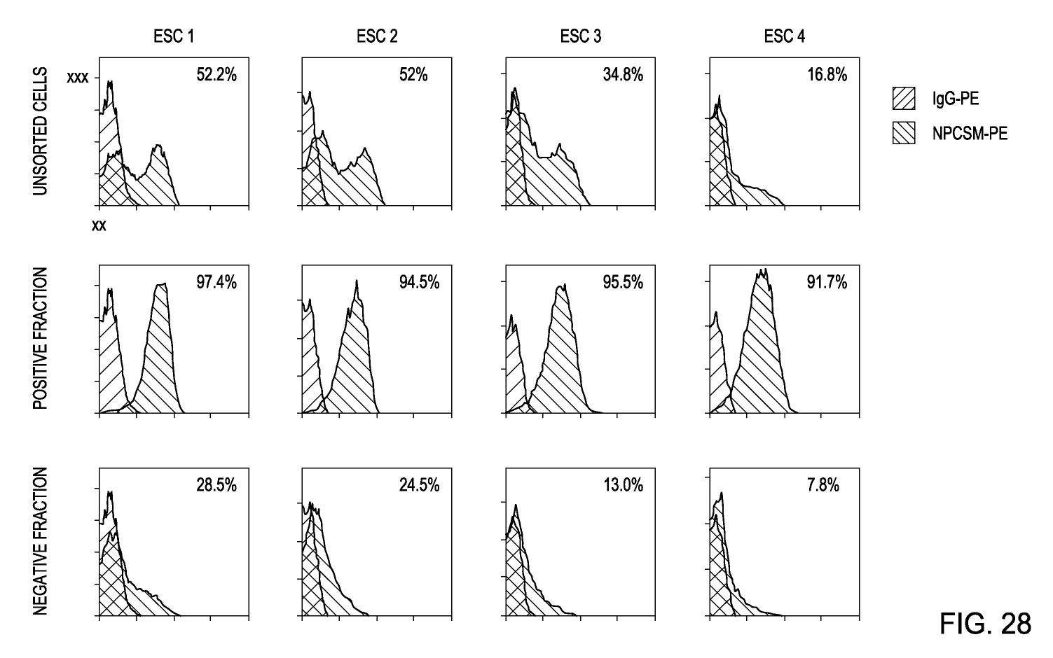

Magnetic sorting efficiently enriches for NPCSM positive cells (e.g., CRCX4+, CRCX7+ or ERBB4+) from four different human ESC lines differentiated toward the MGE-type interneuron lineage. FIG. 28 shows an exemplary set of flow cytometry histogram plots showing percent NPCSM-positive cells in unsorted hESC-derived cultures from four different ESC lines (top row), compared to positive (middle row), and negative (bottom row) sorted fractions.

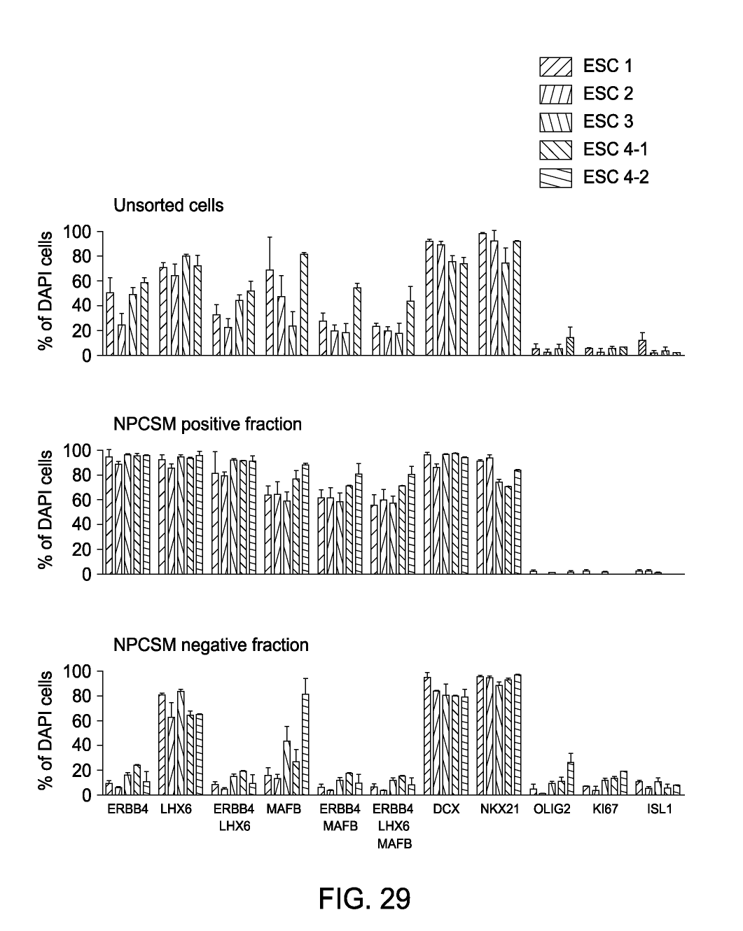

ICC analysis of the unsorted, NPCSM positive and NPCSM negative sorted fractions isolated from hESC-derived cultures shows enrichment of interneuron markers including ERBB4, LHX6 and MAFB and depletion of progenitor cell markers (OLIG2 and Ki67) and projection neuron marker (ISL1) in the NPCSM positive fraction (FIG. 29). The increased or decreased expression of these markers in the hESC-derived neural precursor populations can identify cell populations of interest for transplantation, as they are enriched in cells with the ability to migrate and differentiate into GABA-producing cells in vivo. Such cell populations can be enriched through differentiation, positive selection, or by depletion of cells expressing cell markers not indicative of the neural precursor cells.

The MGE-like cell populations differentiated from hESCs were further characterized by FACS analysis using antibodies to other surface markers depleted in the NPCSM positive population and enriched in the NPCSM negative population. CD98 negative cells purified from NKX2.1:eGFP hESC-derived MGE-like cultures were enriched for DCX, a marker of post-mitotic migratory neurons. In addition, CD271 expression levels increased then declined over time as hESC cells differentiated into MGE-like cultures. Using FACS analysis, CD271 negative cells purified from NKX2.1:eGFP hESC-derived MGE-like cultures were shown to be enriched for DCX, a marker of post-mitotic migratory neurons.

### Example 9: hESC-Derived Neural Precursor Cells of Interest can Engraft into Mouse Brain

The hESC-derived neural precursor cells that were selected as described above (PLEXINA4+ single positive, NPCSM+ single positive, or PLEXINA4+ NPCSM+) were then tested for their ability to migrate and differentiate into GABA-producing cells in vivo. The sorted cells were transplanted into immunodeficient SCID neonate mouse cortex as described above, and allowed to migrate and differentiate in the mouse brain. After one month of engraftment, the human HNA+ cells exhibited marker expression of MGE-type cortical interneurons including DCX, MAFB, LHX6, and GABA. Little or no SP8 expression was detected within grafted cells, indicating that the interneurons were not LGE- or CGE-type interneurons.

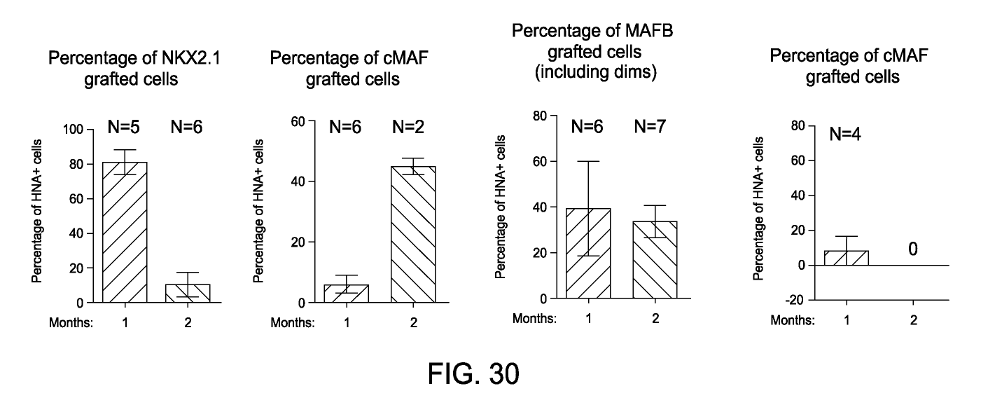

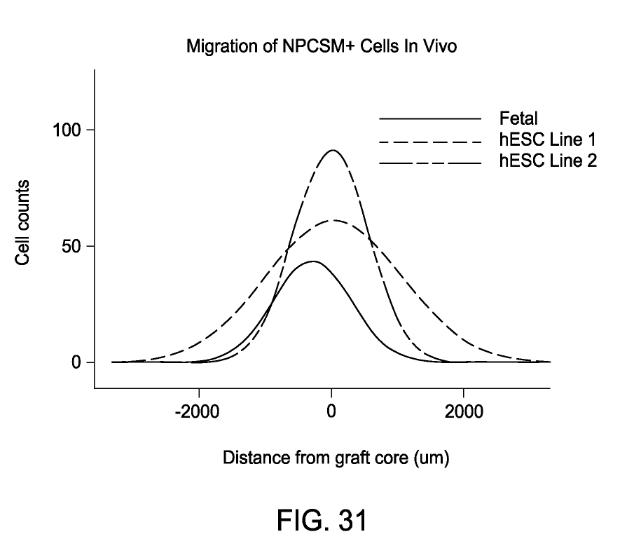

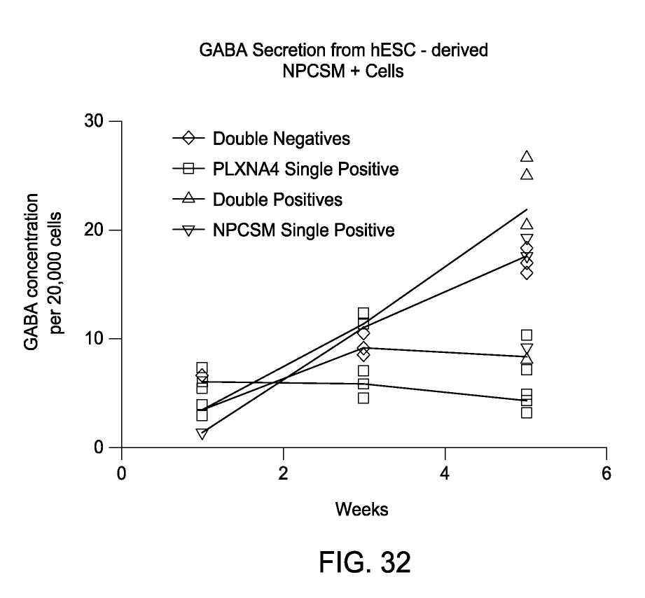

Quantification of human HNA+ cell marker expression by immunohistochemistry at 1 and 2 months post-injection of sorted neural precursors from hESC-derived cultures into immunodeficient mouse cortex shows cortical interneuron maturation by downregulation of NKX2.1 and upregulation of cMAF, maintenance of MAFB expression, and an absence of proliferative cells (Ki67) (FIG. 30). Sorted cells from two different hESC lines injected into mouse cortex yielded robust human cell engraftment and migration throughout the cortex, resembling migratory interneurons post-transplant with NPCSM+ cells from human fetal cortex as discussed previously (FIG. 31). The PLXNA4+ NPCSM+ and NPCSM+ sorted hESC-derived MGE-like neural precursor cells were also shown to secrete elevated levels of GABA upon further culture for 3 to 5 weeks after purification in comparison to NPCSM negative populations (FIG. 32).

### Example 10: hESC-Derived Neural Precursor Cells of Interest can Engraft into Adult Rat CNS

MACS purified NPCSM positive cells from hESC-derived MGE-like cultures were shown to engraft into the adult hippocampus in an immunodeficient rat model of temporal lobe epilepsy (TLE). Upon three weeks of engraftment, the human cells exhibited marker expression of migratory interneurons (DCX and NKX2.1). Little or no SP8 or Ki67 expression markers of LGE and CGE derived interneurons and proliferation, respectively, was detected within grafted cells.

The NPCSM+ MACS-sorted neural precursor cells derived from human ESCs were also grafted into the adult rat spinal cord after contusion injury. One month after transplantation, the mice were sacrificed and their spinal cords analyzed for human cell migration and differentiation. The human HNA+ cells in the spinal cord had migrated and were positive for cortical interneuron markers, including MAFB and LHX6, demonstrating differentiation toward the interneuron lineage.

### Example 11: Treatment of Seizure Disorders with the Neural Precursor Cell Populations of the Invention

The neural precursor cell populations of the invention are examined for their ability to reduce acute and chronic seizures. Restoration or increase of inhibitory interneuron function in vivo is achieved by transplantation of MGE cells into the brain, and such cells were demonstrated to migrate in host neocortex with distributions between 0.75 and 5 mm from the injection site (See U.S. 20090311222, U.S. Pat. No. 9,220,729 and Alvarez-Dolado et al., J Neurosci. 2006 Jul. 12; 26(28):7380-9). The following experiments are performed to demonstrate that the neural precursor cell populations of the invention possess the same ability to migrate and rescue acute seizure disorder in a mouse model of epilepsy.

Spontaneous tonic-clonic seizures have been reported in humans with a dominant-negative missense mutation in KCNA1 or mice with a recessive knockout of Kv1.1/Kcna1 (Zuberi S M et al., Brain. 1999 May; 122:817-25). To monitor spontaneous seizures in these mice, prolonged video-electroencephalography (EEG; see Methods for full description of electrographic phenotypes) is performed. The EEG of Kv1.1-1-mice show severe, generalized electrographic seizures lasting 10-340 seconds and occurring more than once per hour; electrographic seizures or high voltage spiking were never observed in age-matched wild-type siblings. Video monitoring confirmed tonic-clonic, S4 seizure behavior (e.g., tonic arching, tail extension, followed by forelimb clonus, and then synchronous forelimb and hindlimb clonus) during ictal seizure episodes.

Temporal lobe epilepsy (TLE) is a common seizure disorder characterized by spontaneous recurrent seizures, which are debilitating to the patient. Currently, many patients do not respond to anti-epileptic drugs and have limited treatment options such as highly invasive temporal lobe resection. In most cases, even following the surgical resection of epileptic focus, the seizures eventually return. Defects in inhibitory GABAergic signaling are one of the known causes of TLE. Transplantation of GABAergic interneurons into the hippocampus is a promising therapeutic approach to treat TLE patients. Seizures typically involve hyperactivation or overexcitation of neural circuits and impair brain function. The new interneurons shift the excitation/inhibition balance in the brain towards inhibition. To evaluate the therapeutic potential of NPCSM+ interneuron transplants, adult rat and mouse kainate (Rattka et al., Epilepsy Research, 2013, 103, 135-52) and pilocarpine (Borges K et al., Experimental Neurology, 2003, 21-34) models of TLE are used.

To induce TLE-type seizures with repeated low-dose kainate, animals are given IP injections of 5-15 mg/kg of kainate every hour until they develop stage 5 seizures on the Racine scale. The animals are allowed to have seizures for 30-90 minutes before administering 10 mg/kg diazepam (IP) to terminate the seizures. To induce status epilepticus with pilocarpine, the animals are pre-treated with scopolamine (1 mg/kg, 30 min) and then directly injected with 100-500 mg/kg pilocarpine IP. Pretreatment with scopolamine blocks peripheral effects of pilocarpine. Video and EEG recordings and behavioral experiments are used to measure seizure frequency and duration in order to evaluate efficacy and safety of the cell transplants.

The neural precursor cell populations of the invention are concentrated to ˜1,000 cells/nl. The concentrated cell suspensions are loaded into a beveled glass micropipette (Wiretrol 5 μl, Drummond Scientific Company) and mounted on a hydraulic injector. Epileptic animals are anesthetized through hypothermia and positioned in a clay head mold on the injection platform. Using a stereotax, 25-50,000 cells per injection site are injected transcranially into the brain (including but not limited to cortex, striatum, hippocampus, thalamus, amygdala, subiculum, entorhinal cortex) of each animal at 1.0 mm from the midline (sagittal sinus), 2.6 mm from the lambda and 0.3 mm deep from the skin surface.

As reported (Smart 1999; Wenzel 2007; Glasscock 2007), Kv1.1-1-mice exhibit frequent spontaneous seizures starting during the second-to-third postnatal week and do not survive beyond the 8th postnatal week; sudden death is likely due to cardio-respiratory failure associated with status epilepticus. In contrast, Kv1.1-1-mice grafted with the neural precursor cells of the invention on P2 survive well past postnatal week 10 and exhibit a reduction in electrographic seizure activity. The frequency of seizure events is rare compared to un-transplanted mice. Kaplan-Maier survival plots show a clear, and statistically significant, rightward shift for Kv1.1 mutant mice receiving successful transplantation of the neural precursor cell populations of the invention. Similarly, TLE models exhibit a latent phase for several weeks post-status followed by a ramp-up phase of seizure frequency until animals develop >1 spontaneous recurrent seizure per day. Adult animals injected with the neural precursor cells of the invention show significantly reduced spontaneous seizure activity (decreased seizure frequency, duration, and/or severity) as measured by electrographic EEG recording and/or by behavioral seizure analysis.

### Example 12: Treatment of Parkinson's Disease with the Neural Precursors of the Invention

Parkinson's disease (PD) affects approximately 150 per 100,000 people in the United States and Europe. PD is characterized by motor impairment as well as cognitive and autonomic dysfunction and disturbances in mood. Four cardinal features of PD can be grouped under the acronym TRAP: Tremor at rest, Rigidity, Akinesia (or bradykinesia) and Postural instability. In addition, flexed posture and freezing (motor blocks) have been included among classic features of Parkinsonism, with PD as the most common form. Existing treatments can attenuate the symptoms of PD but there is no cure.

The motor symptoms of PD result primarily from the loss of dopamine containing neurons in the substantia nigra compacta (SNc) that extend axonal projections to the striatum and release dopamine (for review see (Litvan et al., 2007, J Neuropathol Exp Neurol. 2007 May; 66(5):329-36). The SNc and the striatum belong to the basal ganglia, a network of nuclei which integrate inhibitory and excitatory signals to control movement. Loss of SNc cells in PD reduces the amount of dopamine release into the striatum, producing a neurotransmitter imbalance that inhibits the output of the basal ganglia and produces hypokinetic signs (for review see DeLong and Wichmann, 2007, Arch Neurol. 2007 January; 64(1):20-4).

It has previously been demonstrated that transplantation of MGE cells can treat the

motor symptoms of Parkinson's disease produced by a reduction of dopaminergic input, a non-dopamine based strategy that modified the circuit activity in the basal ganglia (See U.S. Pat. App. No. 20130202568). Briefly, MGE cells are transplanted into the striatum of rats treated with 6-hydroxydopamine (6-OHDA), a well-established model of PD. This treatment relied on the ability of MGE cells to migrate, functionally integrate, and increase levels of inhibition in the host brain after transplantation. Transplanted MGE cells migrated from the site of injection and dispersed throughout the host striatum. Most MGE transplant cells acquired a mature neuronal phenotype and expressed neuronal and GABAergic markers. In addition, the transplanted cells expressed a variety of markers that are characteristic of striatal GABAergic interneurons such as CB, CR, CB, and Som. Finally, the MGE transplant cells became physiologically mature, integrated into the host circuitry, and improved the motor symptoms of PD in the rat 6-OHDA model. These results indicated that the transplantation of GABAergic interneurons restores balance to neuronal circuitry that has been affected by neurodegenerative diseases such as PD.

Similarly, the neural precursor cell populations of the present invention are useful in the treatment of Parkinson's disease. The neural precursor cells of the invention are transplanted into a well-established animal model of Parkinson's disease, 6-OHDA model. Unilateral lesions of the nigrostriatal projection in rats, using 6-OHDA, leads to the loss of dopaminergic cells in the SNc through retrograde transport, and loss of dopaminergic terminals in the striatum through axonal disruption (Berger et al., 1991, Trends Neurosci. 1991 January; 14(1):21-7.). As a consequence, the distribution of D1 and D2 receptors is altered. Unilateral damage can result in bilateral changes in the SNc (Berger et al., supra). Damage of the nigrostriatal pathway in rats is accompanied by a compensatory increase in the synthesis and release of dopamine from the dopaminegic terminals that remain (Zigmond et al., 1984 Life Sci. 1984 Jul. 2; 35(1):5-18).

Adult female rats are anesthetized with ketamine (90 mg/Kg) and xylazine (7 mg/Kg), and when insensitive to pain, are immobilized within a stereotaxic frame in flat skull position. A two-centimeter mid-sagittal skin incision is made on the scalp to expose the skull. The coordinates for the nigrostriatal bundle are determined based on the computerized adult rat brain atlas (Toga A W et al., 1982 Brain Res Bull. 1989 February; 22(2):323-33). A hole is drilled through the skull at the appropriate coordinates, and a glass capillary micropipette stereotaxically advanced so that the internal tip of the pipette is located within the nigro-striatal pathway. The micropipette has a 50 μm diameter tip and is filled with a solution of 6-OHDA, 12 gr/3 μl in 0.1% ascorbic acid-saline. The 6-OHDA is injected into the right nigro-striatal pathway at a rate of 1 μl/minute. The micropipette is kept at the site for an additional 4 minutes before being slowly withdrawn. The skin incision is closed with stainless steel wound clips. Each animal is injected with 6-OHDA on the right side only, producing hemi-Parkinsonian rats.

6-OHDA lesions are induced on experimental day 1 and behavioral tests performed on weeks 3 and 5. In rats selected for grafting, neural precursor cells of the invention are transplanted on week 6, and behavioral tests are repeated on weeks 9, 11, 14 and 18. To evaluate the success of surgery, a subset of animals (n=5) are perfused 4 weeks after lesion and the SNc stained for tyrosine hydroxylase immunoreactivity (TH-IR), a limiting enzyme in the synthesis of dopamine, in order to label dopaminegic cells. In successful surgeries, the side of the SNc ipsilateral to the 6-OHDA injection does not show TH-IR, while the contralateral side has numerous TH+ cells. To evaluate the 6-OHDA surgeries in vivo, behavioral testing is performed as described below.

Three injections are performed along the rostro-caudal axis of the striatum, and cells are deposited at three delivery sites along the dorsal-ventral axis at each injection site, starting with the most ventral site first and then withdrawing the injection pipette dorsally to perform the second and third injections. Approximately 400 nl of cell suspension is injected at each delivery site, and a total of 3.6 μl of total cell suspension is injected in each striatum.

Behavioral Tests are used to the ability of the neural precursor cell transplantation to ameliorate the behavioral symptoms of 6-OHDA lesioned rats. Three behavioral tests are performed before and after neural precursor cell transplantation: rotation under apomorphine, change in the length of stride, and maximum path width. 6-OHDA lesioned rats that receive neural precursor cell transplants exhibit behavioral improvements including improvement in the apomorphine rotational test, an increase on the length of stride, and a normalized gait. These behavioral and movement changes indicate a general improvement of the motor symptoms of PD animals after transplantation of the neural precursor cells of the invention.

The first behavioral test is rotation under apomorphine. Apomorphine binds to dopamine receptors expressed by host striatal neurons, which causes rotation in the 6-OHDA rat (Ungerstedt and Arbuthnott, 1970, Brain Res. December 18; 24(3):485-93). As previously shown, upon apomorphine administration, unilaterally 6-OHDA lesioned rats rotate significantly more to the contralateral side (with respect to the lesioned side) than the ipsilateral side compared to control rats that rotate approximately equally in both directions. Apomorphine stimulates dopaminergic receptors directly, preferentially on the denervated side due to denervation induced dopamine receptor supersensitivity, causing contralateral rotation (Ungerstedt and Arbuthnott, 1970). There is a threshold of damage that must be reached in order to produce maximal rotation behavior after apomorphine administration (Hudson et al., 1993). The abnormal behavior of hemi-Parkinsonian rats is directly related to the amount of DA cell loss. When there is less than 50% dopamine depletion in the striatuma significant change in rotation behavior after apomorphine injection was not observed, due to compensatory mechanisms in the striatum.

Each test rat is injected with the dopamine agonist apomorphine (0.05 mg/kg, IP) to produce contralateral rotational behavior in 6-OHDA treated rats. Drug-induced rotations are measured in an automated rotometer bowl (Columbus Instruments, Ohio, Brain Research, 1970, 24:485-493). After intraperitoneal injection of apomorphine, the animals are fitted with a jacket that is attached via a cable to a rotation sensor. The animals are placed in the test bowl and the number and direction of rotations is recorded over a test period of 40 minutes. This test is administered to each rat to verify and quantify the efficacy of the intracranial 6-OHDA-infusion. For the grafting experiment only those 6-OHDA rats that rotated at least four times more to the contralateral than to the ipsilateral side of the injection are selected.

After neural precursor cell transplantation, there is a significant reduction in the number of contra-lateral turns in the transplanted 6-OHDA lesioned rats compared to non-transplanted 6-OHDA controls. This effect is observed at all experimental times beginning with week 9 through to at least 18 weeks. The performance of sham-transplanted 6-OHDA rats is undistinguishable from non-transplanted 6-OHDA rats, indicating that the neural precursor cells, and not the transplantation procedure, is responsible for the motor improvement of MGE-transplanted 6-OHDA rats.

The second behavioral effect of the transplanted neural precursors of the invention on 6-OHDA lesioned rats is a change in the length of stride. A test animal is placed on a runway 1 m long and 33 cm wide with walls 50 cm high on either side. The runway is open on the top, and was situated in a well-lit room. A dark enclosure is placed at one end of the runway, and rats are free to enter the enclosure after traversing the runway. Rats are trained to run down the runway by placing them on the runway at the end opposite to the dark enclosure. The practice runs are repeated until each rat runs the length of the runway immediately upon placement in the runway. The floor of the runway is covered with paper. At the start of each test, the animals' rear feet are dipped in black ink before being placed at the beginning of the runway. The test is repeated for each rat and the length of stride for each test is measured to obtain an average stride length for each rat.

The average stride length is compared across groups. 6-OHDA rats display impairments in the posture and movement of the contralateral limbs. They compensate by supporting themselves mainly on their ipsilateral limbs, using the contralateral limb and tail for balance, and by disproportional reliance on their good limbs to walk. The good limbs are responsible for both postural adjustments and forward movements and they shift the body forward and laterally (Miklyaeva, 1995, Brain Res. 1995 May 29; 681(1-2):23-40). The bad limb produces little forward movement, and as a consequence the length of step is shorter in 6-OHDA rats than in control rats.

The lesioned rats display a significantly shorter stride than the stride length of control rats. After neural precursor cell transplantation, the stride length of the 6-OHDA rats increases and by week 9 reached values similar to those of control rats. The increase in the length of stride is maintained after 11 and 14 weeks. The stride length of 6-OHDA rats that receive a sham transplant does not change, and is not significantly different from that of 6-OHDA rats that received no treatment.

The third behavioral effect of the transplanted neural precursors of the invention on 6-OHDA lesioned rats is the maximum path width traveled by the rats as they descend a runway. 6-OHDA animals have a path width that is significantly wider than the control rats.

Normal control rats run straight down the runway to the enclosure at the end. In 6-OHDA rats, however, the limb impairment produces a wandering path that zig-zagged from side to side, and as a consequence the pathway followed by the 6-OHDA rats is wider than normal. The maximum width of the path for control and experimental groups are compared to determine the effect of the neural precursor cell population on the rats' ability to descend the runway.

The path width of 6-OHDA rats that received neural precursor cell transplants decreases and by week 11 is similar to that of the unlesioned control animals. The path of sham transplanted 6-OHDA rats is not significantly different from that of 6-OHDA rats, indicating that it is the cellular transplant responsible for a substantial improvement in the gait of 6-OHDA lesioned rats.

### Example 13: Treatment of Spinal Cord Injury (SCI) with the Neural Precursors of the Invention

MGE cells have previously been described with the ability to ameliorate certain pathologies associated with spinal cord injury (see, e.g., See e.g., U.S. Pat. No. 9,220,729 and U.S. Pat. App. 20130202568). Neural precursor cell populations are implanted into the uninjured cord of rodents to assess their integration into the local circuitry and also into contused and transected spinal cords. Both contusion and transection are studied in order to assess mild (contusion) and moderate (transection) levels of spasticity.

Genetically modified and wild-type mice are anesthetized with Avertin supplemented with isoflurane or isoflurane only. The skin over the middle of the back is shaved. The shaved area is disinfected with Clinidine. All surgical tools are soaked overnight in Cidex prior to their use. Lubricating ophthalmic ointment is placed in each eye. Animals are placed on a warming blanket to maintain temperature at 37° C. A dorsal midline incision, approximately 1 cm in length is made using a scalpel blade. The spinous process and lamina of T9 are identified and removed. A circular region of dura, approximately 2.4 mm in diameter, is exposed. At this point the animal is transferred to the spinal cord injury device that is about 5 feet from the surgical area. Small surgical clamps are placed on a spine rostral and a spine caudal to laminectomy site to stabilize the vertebral column. Thereafter, a 2-3 g weight is dropped 5.0 cm onto the exposed dura. This produces a moderate level of spinal cord injury. Immediately after injury, the animal is removed from the injury device and returned to the surgical area. A small, sterile suture is placed in the paravertebral musculature to mark the site of injury. The skin is then closed with wound clips and the animal recovered from the anesthesia. The entire surgical procedure is completed within 45 to 60 minutes.

Behavioral tests are used to determine the ability of the neural precursor cell transplantation to improve the physiological impairment associated with SCI similar to the behavioral symptoms of 6-OHDA lesioned rats. Five behavioral tests are performed before and after neural precursor cell transplantation: open field testing, grid walking, foot placement, beam balance, and the inclined plane test.

The first behavioral test is open field testing, which involves testing animals at 3 days post injury and weekly thereafter until time of euthanasia at 42 days. Locomotor testing consists of evaluating how animals locomote in an open field. This open field walking score measures recovery of hindlimb movements in animals during free open field locomotion as described by Basso et al. A score of 0 is given if there is no spontaneous movement, a score of 21 indicate normal locomotion. Plantar stepping with full weight support and complete forelimb-hindlimb coordination is reached when an animal shows a score of 14 points. A modified version of the BBB score is used to determine if the sequence of recovering motor features is not the same as described in the original score. If this is observed, points for the single features are added independently. For example, for a mouse showing incomplete toe clearance, enhanced foot rotation and already a ‘tail-up’ position, one additional point is added to the score for the tail position.

The mice are tested preoperatively in an open field, which is an 80.times.130-cm transparent plexiglass box, with walls of 30 cm and a pasteboard covered non-slippery floor. In postoperative sessions two people, blinded to the treatments, will observe each animal for a period of 4 min. Animals that exhibit coordinated movement, based upon open field testing, are subjected to additional tests of motor function as follows.

The second behavioral test used to assess the effect of the neural precursor cell transplantation on rats with SCI is grid walking. Deficits in descending motor control are examined by assessing the ability of the animals to navigate across a 1 m long runway with irregularly assigned gaps (0.5-5 cm) between round metal bars. The bar distances are randomly changed from one testing session to the next. The animals are tested over a period of 5 days, beginning 1 to 2 weeks prior to euthanasia.

Crossing this runway requires that animals accurately place their limbs on the bars. In baseline training and postoperative testing, every animal will cross the grid for at least three times. The number of footfalls (errors) are counted in each crossing and a mean error rate is calculated. If an animal is not able to move the hindlimbs, a maximum of 20 errors are given. The numbers of errors counted are also rated in a non-parametric grid walk score: 0-1 error is rated as 3 points, 2-5 as 2 points, 6-9 as 1 point and 10-20 footfalls as 0 points.

The third behavioral test used to assess the effect of the neural precursor cell transplantation on rats with SCI is foot placement Footprint placement analysis is modified from De Medinaceli et al. The animal's hind paws are inked, for example, with watercolor paint that can easily be washed off, and footprints are made on paper covering a narrow runway of 1 m length and 7 cm width as the animals traverse the runway. This ensures that the direction of each step is standardized in line. A series of at least eight sequential steps are used to determine the mean values for each measurement of limb rotation, stride length and base of support. The base of support is determined by measuring the core to core distance of the central pads of the hind paws. The limb rotation are defined by the angle formed by the intersection of the line through the print of the third digit and the print representing the metatarsophalangeal joint and the line through the central pad parallel to the walking direction. Stride length are measured between the central pads of two consecutive prints on each side.

To include animals with incomplete weight support in early postoperative testing sessions, a 4-point scoring system is also used: 0 points is given for constant dorsal stepping or hindlimb dragging, i.e. no footprint is visible; 1 point is counted if the animal has visible toe prints of at least three toes in at least three footprints; 2 points are given if the animal showed exo- or endo-rotation of the feet of more than double values as compared to its own baseline values; 3 points are recorded if the animal showed no signs of toe dragging but foot rotation; 4 points are rated if the animal showed no signs of exo- or endo-rotation (less than twice the angle of the baseline values). These animals are tested over a period of 5 days, beginning 1 to 2 weeks prior to euthanasia.

The fourth behavioral test used to assess the effect of the neural precursor cell transplantation on rats with SCI is beam balance. Animals are placed on a narrow beam, and the ability to maintain balance and/or traverse the beam is evaluated. These animals are tested over a period of 5 days, beginning 1 to 2 weeks prior to euthanasia. The narrow beat test is performed according to the descriptions of Hicks and D'Amato. Three types of beams are used as narrow pathways: a rectangular 2.3 cm wide bean, a rectangular 1.2 cm wide beam and a round dowel with 2.5 cm diameter. All beams are 1 m long and elevated 30 cm from the ground. After training, normal rats are expected to be able to traverse the horizontal beams with less than three footfalls. When occasionally their feet slipped off the beam, the animals are retrieved and repositioned precisely.

A scoring system is used to assess the ability of the animals to traverse the beams: 0 is counted as complete inability to walk on the bean (the animals fall down immediately), 0.5 is scored if the animal is able to traverse half of the beam, 1 point is given for traversing the whole length, 1.5 points when stepping with the hindlimbs is partially possible, and 2 points is noted for normal weight support and accurate foot placement. If the scores of all three beams are added, a maximum of 6 points can be reached.

The final behavioral test used to assess the effect of the neural precursor cell transplantation on rats with SCI is the inclined plane test. Animals are placed on a platform that can be raised to varying angles. The ability to maintain position at a given angle is determined. These animals are tested over a period of 5 days, beginning 1 to 2 weeks prior to euthanasia. Animals are placed on an adjustable inclined plane constructed as described (Rivlin and Tator, 1977, J Neurosurg. 1977 October; 47(4):577-81). The slope is progressively increased every 20 seconds noting the angle at which the mouse could not maintain its position for 5 seconds. The test is repeated twice for each mouse and the average angle is recorded. In the inclined-plane test, recovery from motor disturbance is assessed before, and again at 1, 7, 14, and 21 days after the injury. The maximum inclination of the plane on which the rats could maintain themselves for 5 seconds without falling is recorded.

Each of these tests described above takes less than 5 minutes. These various tests are designed such that if animals fall from the testing apparatus, they either land on padded flooring or the distance fallen is sufficiently limited (less than 6 inches) that the animals are not be harmed.

### Example 14: Treatment of Spasticity with the Neural Precursors of the Invention

Spasticity is a common disorder in patients with injury of the brain and spinal cord. The prevalence is approximately 65-78% of patients with spinal cord injury (Maynard et al. 1990), and around 35% in stroke patients with persistent hemiplegia (Sommerfeld et al. 2004). Reflex hyperexcitability develops over several months following human spinal cord injuries in segments caudal to the lesion site. Intractable spasticity is also a common source of disability in patients with multiple sclerosis. Symptoms include hypertonia, clonus, spasms and hyperreflexia. Bladder spasticity is also a common occurrence in the elderly, women in or following pregnancy, and during menopause.

While the precise mechanisms responsible for the development of spasticity are not fully understood, grafting MGE cells into the affected regions has been shown to ameliorate spasticity in mouse model of spinal cord injury. (See e.g., U.S. Pat. No. 9,220,729 and U.S. Pat App. 20130202568). Similarly, the neural precursor cell populations of the present invention are useful in the treatment of spasticity. The neural precursor cell populations of the invention are transplanted into an animal model of SCI. Mice receiving neural precursor cell populations in the grey matter of the spinal cord exhibit improved bladder function, fewer uninhibited bladder contractions and less residual urine, as compared to control animals that received dead cell/vehicle injections or no injection.

### Example 15: Treatment of Neuropathic Pain with the Neural Precursors of the Invention

In addition to the above-described experiments, transplantation of neural precursor cells can ameliorate neuropathic pain. In particular, the neuronal precursor cells can be transplanted into animal model studies using injury-induced neuropathic pain using the spared nerve injury (SNI) model as described previously (Shields et al., 2003). This model is produced by transection of two of the three branches of the sciatic nerve on one side of the animal resulting in prolonged mechanical hypersensitivity (Shields et al., J Pain. 2003 October; 4(8):465-70).

All transplants are performed on male mice (6-8 weeks old). The ZW and ZWX mice were described previously (Braz and Basbaum, 2009, Neuroscience. November 10; 163(4):1220-32). To generate double transgenic ZWX-NPY mice, ZWX mice are crossed with mice that express Cre recombinase in NPY expressing neurons (DeFalco et al., 2001, Science. March 30; 291(5513):2608-13). To generate Per-ZW mice, the ZW mice are crossed with Peripherin-Cre mice (Zhou et al., 2002, FEBS Lett July 17; 523(1-3):68-72).

For transplantation, 6 to 8 week old mice (naïve or one week after SNI) are anesthetized by an intraperitoneal injection of ketamine (60 mg/kg)/xylazine (8 mg/kg). A dorsal hemilaminectomy is made at the level of the lumbar enlargement to expose 2 segments (˜1.5-2 mm) of lumbar spinal cord, after which the dura mater is incised and reflected. A cell suspension containing 5×10′ neural precursor cells is loaded into a glass micropipette (prefilled with mineral oil). The micropippete is connected to a microinjector mounted on a stereotactic apparatus. The cell suspension injections are targeted to the dorsal horn, ipsilateral to the nerve injury. The control groups are injected with an equivalent volume of DMEM medium. The wound is closed and the animals are allowed to recover before they are returned to their home cages. Animals are killed at different times post-transplantation (from 1 to 5 weeks).

Mechanical sensitivity is assessed by placing animals on an elevated wire mesh grid and stimulating the hindpaw with von Frey hairs. The up-down paradigm (Chaplan et al., 1994 J Neurosci Methods. July; 53(1):55-63) is used to define threshold. Animals are tested 3 times, once every other day before surgery to determine baseline threshold, and once 2 days after surgery, to assess the magnitude of the mechanical allodynia. Only animals that display at least a 50% drop of the mechanical withdrawal threshold are included in the transplantation group or the medium injection (control group) groups. Behavioral testing takes place on days 7, 14, 21 and 28 after transplantation or medium injections. For the behavioral tests, the investigator is blind to treatment (cell medium or cell population transplantation). Thermal hyperalgesia assays (hot/cold plate, Hargreaves, and tail flick) are also used to measure pain sensitivity.

The test animals are also subjected to the rotorod test and a hindpaw injury assay, an established assay for detecting neuropathic pain in mice. For the accelerating rotorod test, the rats are trained to stay on the rotating spindle of the rotarod in three sessions with three trials per session at the beginning and a single trial after the rat can stay more than 60 seconds on the spindle. The acceleration of the rotarod is set to automatically increase from 4 to 40 rpm within 5 min, and trials automatically end when the animals fall off the spindle. In the tail flick assay, 10 l of a 1% formalin solution (Sigma, St. Louis, Mo.) is injected into the hindpaw of medium or neural precursor cell transplanted mice, ipsilateral to the transplanted side. The mice are scored for the total time spent flinching or licking the injected hindpaw (in 5 min bins). The behavioral scores are made by an experimenter blinded to treatment group.

This transplantation results in a dramatic reduction of the mechanical threshold (von Frey) ipsilateral to the injury side. A significant difference between control and neural precursor cell population-transplanted groups is first detected two weeks post-transplantation (23 days post-SNI), similar to the time predicted to be necessary for the transplanted cells to differentiate into neurons and integrate into the host circuitry. The magnitude of the recovery continues to improve and pain thresholds return to pre-injury baseline levels 4 weeks after transplantation of the neural precursor cells of the invention.

Importantly, none of the transplanted animals exhibit signs of motor impairment. Furthermore, mice in both groups are found to walk on a rotating rod for the observation period. In the hindpaw injury assay, transplantation of neural precursor cells into the grey matter of the spinal cord results in a decrease in pain in comparison with mice receiving injection of medium. Five mice are assessed for each study group, and the mice receiving the injection of neural precursor cells demonstrate a statistically significant decrease in neuropathic pain as compared to the medium only group.

Similar results are achieved when injecting the neural precursor cells of the invention into the spinal cords of animal models of SCI. SCI induces chronic pain, both mechanical allodynia and thermal hyperalgesia. Neural precursor cells injected into the spinal cord in the acute, subacute, and/or chronic phase post-injury ameliorate chronic neuropathic pain.

### Example 16: Treatment of Cognitive Defects in Alzhelmer's Disease with the Neural Precursors of the Invention

The methods of the invention can also be used in the treatment of Alzheimer's diseases to ameliorate the impairment of learning and memory in these patients. Neural precursor cells can be transplanted into the hilus of apoE4-KI mice, or into models of familial Alzheimer's disease (FAD), to demonstrate the rescue of apoE4-induced cognitive defects, as well as seizures, as previously described (Tong L M et al., J. Neuroscience 34(29):9506-9515). The transplantation of the neural precursor cells of the invention results in functional maturation and integration of transplant-derived GABAergic interneurons in the hippocampus, and rescue of apoE4-induced cognitive deficits in adult mice.

Female apoE4-KI and apoE3-KI mice at 14 months of age and apoE4-KI/hAPP_{FAD} mice at 10 months of age are anesthetized with 80 μl of ketamine (10 mg/ml) and xylazine (5 mg/ml) in saline solution and maintained on 0.8-1.0% isoflurane. Neural precursor cell suspensions (600 cells/nl) are loaded into a 60 μm tip diameter, 300 beveled glass micropipette needles (Nanoject, Drummond Scientific Company). Bilateral rostral and caudal stereotaxic sites are drilled with a 0.5 mm microburr (Foredom, Fine Science Tools), and hilar transplantation is performed at four sites. At each transplantation site, an estimated 20,000 neural precursor cells are introduced. Control transplant mice receive an equivalent volume of heat-shocked dead neural precursor cells, which are generated by four alternating cycles of 3 min at 55° C. and 3 min in dry ice before centrifugation collection. (Alvarez-Dolado et al., 2006, J Neurosci. 2006 Jul. 12; 26(28):7380-9.; Baraban et al., 2009, Proc Natl Acad Sci USA. September 8; 106(36):15472-7.; Southwell et al., 2010, Science. February 26; 327(5969): 1145-8).

To assess the ability of the transplanted neural precursor cell populations to improve cognition in the Alzheimer's models, behavioral tests are performed for cell-transplanted and control-transplanted mice at 70-80 DPT. The Morris water maze (MWM) test is conducted in a pool (122 cm in diameter) with room temperature water (22-23° C.) with a 10 cm² platform submerged 1.5 cm below the surface of opaque water during hidden trials (Andrews-Zwilling et al., 2010, J Neurosci. October 13; 30(41): 13707-17.; Leung et al., 2012, PLoS One. 7(12):e53569). Mice are trained to locate the hidden platform over four trials per day on hidden platform days 1-5 (HD1-5), where HD0 is the first trial on the first day, with a maximum of 60 seconds per trial. Each memory trial is conducted for 60 seconds in the absence of the platform at 24, 72, and 120 hours after the final learning session. Memory is assessed as the percentage of time spent in the target quadrant that contained the platform during the learning trials compared with the average percentage of time spent in the nontarget quadrants.

For visible trials, a black and white-striped mast (15 cm high) marked the platform location. The platform location and room arrangement remains constant throughout the assay with the exception of moving the platform during the visible trials. Speed is calculated by distance traveled divided by trial duration. Performance is objectively monitored using EthoVision video-tracking software (Noldus Information Technology).

The open field test assesses habituation and general activity behavior by allowing the mice to explore a new, but empty, environment (Andrews-Zwilling et al., 2012, PLoS One. 7(7):e40555.). After at least 2 hours of room habituation, mice are placed in an odor-standardized chamber cleaned with 30% EtOH for 15 min. Activity behavior is monitored and analyzed by software from San Diego Instruments. The elevated plus maze evaluates anxiety and exploratory behavior by allowing mice to explore an open, illuminated area (open arm) or hide in a dark, enclosed space (closed arm; Bien-Ly et al., 2011, Proc Natl Acad Sci USA. March 8; 108(10):4236-41). Here, mice are placed in an odor standardized maze cleaned with 30% EtOH for 10 min after at least 2 hours of room habituation. Behavior is analyzed by infrared photo cells interfacing with Motor Monitor software (Kinder Scientific).

The transplantation of the neural precursor cells into the hilus of apoE4-KI mice demonstrates not only the ability to improve the cognitive deficits observed in these mice, but also the ability to produce functionally integrated interneurons in the presence of apoE4 and Aβ accumulation.

### Example 17: Transplantation of Neural Precursor Cell Populations of the Invention for the Treatment of Stroke-Induced Impairments

The ability for the neural precursor cells of the invention to improve locomotion and coordination in a subject with a traumatic brain injury, such as stroke, is tested using the middle cerebral artery occlusion (MCAO) model of stroke. Briefly, Sprague-Dawley (SD) adult rats (275-310 g, Charles River Laboratories, Wilmington, Mass.) are subjected to 1.5 hours of transient MCAO by intraluminal suture, as previously described (Daadi, M. et al., PLoS ONE 3(2):e1644; 200.). The elevated body swing test (EBST) is used to assess body asymmetry after MCAO, as previously described (Borlongan, C. V. et al., J. Neurosci. 15(7) Pt. 2):5372-5378; 1995). Animals are suspended by tail and the frequency of initial head swings contralateral to the ischemic side is counted in 20 trials and represented as percent of total. Ischemic rats with more than 75% biased swing are used in the study. Two weeks after the ischemic lesion, 2 μl of the neural precursor cell suspension, at a concentration of 50,000 cell/μl, are stereotaxically transplanted into four sites within the lesioned striatum and cortex. Rats subjected to ischemia and transplanted with the vehicle are used as controls.

To investigate the ability of the neural precursor transplantation to influence rewiring of the stroke-damaged side in the MCAO stroke model, axons originating from the intact hemisphere can be labeled by injecting BDA into the right sensorimotor cortex 3 weeks after neural precursor cell transplantation. Three weeks after cell transplantation, three randomly selected animals from each of transplanted and vehicle-treated groups are anesthetized and placed in the strereotaxic apparatus. After craniotomy, 0.5 μl of biotinylated dextran amine [BDA, 10,000 molecular(MW), Molecular Probes, Eugene Oreg.; 10% w/v solution in sterile PBS] is injected stereotaxically into the sensorimotor cortex opposite to the stroke lesion site. The scalp is then closed and the animal returned to its cage. Animals are sacrificed 1 week after BDA injection. The quantitative analysis of BDA-labeled terminals, normalized to the total number of labeled somas at the injections site (see Materials and Methods for details), reveal an increase in the transplanted side.

To determine if the neural precursor transplantation has an effect on motor function following stroke, the test animals are subjected to two motor behavioral tests, the rotorod test and the EBST. Baseline motor behavioral assessment of all animals is performed before and 2 weeks after the ischemic lesion, and 4 weeks after transplantation. For the accelerating rotorod test, the rats are trained to stay on the rotating spindle of the rotarod in three sessions with three trials per session at the beginning and a single trial after the rat can stay more than 60 seconds on the spindle. The acceleration of the rotarod is set to automatically increase from 4 to 40 rpm within 5 minutes, and trials automatically end when the animals fall off the spindle. For the EBST, animals are suspended by tail and the frequency of head swings contralateral to the ischemic side is counted in 20 trials and represented as percent of total as described above. The neural precursor transplanted rats improve in their locomotion and motor coordination with a significant improvement in both tests.

To determine if the neural precursor cell transplants have an effect on stroke or traumatic brain injury-induced seizures, video/EEG monitoring of seizure frequency, severity, and duration is performed. As described above, the neural precursor transplanted animals have significantly reduced seizure activity.

### Example 18: Transplantation of Neural Precursor Cell Populations into a Model of Autism

The methods of the invention can also be used in the treatment of autism spectrum disorder to ameliorate behaviors such as social deficits and learning deficiencies in these patients. BTBR T⁺ Itpr3^{tf}/J mice (BTBR mice) are a well-studied model of idiopathic autism (Defensor, E.B., Pearson et al., (2011). Behav. Brain Res. 217, 302-308. McFarlane, H. G. et al., (2008). Genes Brain Behav. 7, 152-163; Yang, M., et al. (2012), Physiol. Behav. 107, 649-662.). BTBR mice have a reduced level of inhibitory neurotransmission mediated by GABA_{A} receptors in the hippocampus compared to the control strain C57BL/6J, which may contribute to their autistic-like behaviors. Han et al., Neuron 2014; 81:1282-1289. The transplantation of the neural precursor cells of the invention and the resulting functional maturation and integration of transplant-derived GABAergic interneurons in the hippocampus can rescue autism-like behaviors in the BTBR mice receiving transplants of the neural precursor cells.

Female BTBR mice at 14 months of age are anesthetized with 80 μl of ketamine (10 mg/ml) and xylazine (5 mg/ml) in saline solution and maintained on 0.8-1.0% isoflurane. Neural precursor cell suspensions (600 cells/nl) are loaded into a 60 μm tip diameter, 300 beveled glass micropipette needles (Nanoject, Drummond Scientific Company). Bilateral rostral and caudal stereotaxic sites are drilled with a 0.5 mm microburr (Foredom, Fine Science Tools), and striatal transplantation is performed at four sites. At each transplantation site, an estimated 20,000 neural precursor cells are introduced. Control transplant mice receive an equivalent volume of heat-shocked dead neural precursor cells, which are generated by four alternating cycles of 3 min at 55° C. and 3 min in dry ice before centrifugation collection. (Alvarez-Dolado et al., 2006; Baraban et al., 2009; Southwell et al., 2010).

The behavioral tests administered to measure the effect of the transplantation of neural precursor cells on the autism-like behaviors of the BTBR mice include the three-chamber social interaction test, which measures the time of interaction of the test mouse with a stranger mouse versus a novel object, and the open field test, which measures anxiety-related behaviors. The BTBR mice receiving the neural precursor cell transplantation exhibit a higher interaction ratio, higher reciprocal interaction times, and more frequent nose-to-nose sniff time in the three-chamber social interaction test and/or the open field reciprocal social action test than the control mice. The BTBR mice receiving the neural precursor cell transplantation also display decreased hyperactivity in the open field test, measured as the total distance moved towards the center of the open field, and decreased stereotyped circling behaviors. These results indicate that the increased inhibitory interneuron activity is able to decrease the behavioral defects in the BTBR mice.

The transplantation of the of neural precursor cells is also able to ameliorate the cognitive defects exhibited by the BTBR mice. BTBR mice are known to have impaired fear memory (MacPherson, P et al (2008). Brain Res. 1210, 179-188), and context-dependent fear conditioning can be used to measure cognitive deficits associated with autism. Both short term (30 min) and long term (24 hour) memory performance in fear conditioning to the spatial context is improved in the BTBR mice 9 weeks after receiving the neural precursor cell transplantation

To test spatial learning and memory in the absence of fear, a Barnes circular maze test is performed, in which mice rapidly escape a brightly lit field by learning the location of a hole with a dark refuge at it periphery. BTBR mice 9 weeks post-transplantation display a significantly reduced performance time after repeated training sessions in comparison to the BTBR control counterparts.

### Example 19: Transplantation of Neural Precursor Cell Populations into a Model of Psychosis

The methods of the invention can also be used in the treatment of psychosis disorders, such as schizophrenia, to ameliorate behaviors associated with neural dysregulation in these patients. The cyclin D2 knockout (Ccnd2−/−) mouse model display cortical PV+ interneuron reductions associated with adult neurobehavioral phenotypes relevant to psychosis, including increased hippocampal basal metabolic activity, increased midbrain DA neuron activity, augmented response to AMPH, and disruption of cognitive processes that recruit and depend on the hippocampus.. Ccnd2−/− mice have several neurophysiological and behavioral phenotypes that would be predicted to be produced by hippocampal disinhibition, including increased ventral tegmental area dopamine neuron population activity, behavioral hyper-responsiveness to amphetamine, and impairments in hippocampus-dependent cognition. See, e.g., Gilani et al. Proc Natl Acad Sci USA. 2014 May 20; 111(20):7450-5.

Ccnd2 knockout mice are maintained on a C57BL/6J background. Neural precursor cell populations are produced as described herein, and an appropriate excipient is added to facilitate transplantation. For control transplants, the neural precursor cells are killed by repeated freeze-thaw cycles. Live cells at an average density of 30,000 live cells per microliter or control (killed-cell) suspensions are injected bilaterally into the caudoventral hippocampal CA1 of 6- to 8-week-old mice by using a glass pipette (50-μm outer-tip diameter) connected to a nano-injector.

Spontaneous and amphetamine-induced locomotor activity is measured in 17×17-inch open field boxes under standard lighting conditions. Mice are placed in open field for 30 minutes, after which, amphetamine (2 mg/kg dissolved in isotonic saline at 0.2 mg/mL) or saline is injected via intraperitoneal injection. Distance traveled is measured for another 60 minutes. A mixed ANOVA design with genotype and drug as factors, and time (before or after injection) as the repeated measure, is used as described (Id.). This analysis is followed with planned Student t test comparisons of genotypes within drug condition separately for baseline and post-injection locomotion. Contextual fear conditioning methods are adapted from previous studies (e.g., Saxe M D, et al. (2006) Proc Natl Acad Sci USA 103(46):17501-17506; Quinn J J, et al. (2008) Hippocampus 18(7):640-654).

Briefly, mice are acclimated to the testing room 1 hour before training. The training/testing apparatus is a chamber with shock grid floors placed within a sound-attenuating chamber. The inner chamber features a distinctive combination of visuospatial, tactile, and odor cues, which together define the context. On the day of training, mice are placed in one context (“training context”) and the CS+ consisting of a tone (85 dB, 20 s duration, 4.5 kHz) is presented at 300, 470, 580, 670, and 840 seconds. During the last second of each tone, a 0.7-mA scrambled current is delivered through the floor grid (US+). Mice are removed from the training context 140 seconds following the last CS−US presentation. Twenty-four hours later, mice are placed in a novel context and the tone CS+ is presented without shock at 300, 410, 580, 670, and 830 seconds. Six hours after the tone CS+ retrieval test, mice are placed in the training context for 600 seconds. Conditioned freezing, defined as absence of movement except for respiration, is quantified for the following epochs: (i) during the first presentation of the tone-CS+, (ii) for the 40-100 seconds following the offset of CS+ presentations 2-5 (posttone freezing; averaged for all five tones), and (iii) in the training context. Data are analyzed with a mixed ANOVA with retrieval phase as the repeated measure and genotype as the between subjects factor.

The neural precursor transplanted cells improve in their locomotion and motor coordination with a significant improvement in both tests.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. All references cited herein are incorporated by their entirety for all purposes. In the claims that follow, unless the term “means” is used, none of the features or elements recited therein should be construed as means-plus-function limitations pursuant to 35 U.S.C. § 112, 6.

What is claimed is:

1. A method of generating a population of neural cells, comprising:
isolating cells from mammalian brain tissue having increased expression of two or more markers upregulated in neural cells;
wherein the isolated cell population comprises neural cells.

2. The method of claim 1, wherein the cell population comprises neural cells capable of forming GABA-producing neurons.

3. The method of claim 1, wherein the cell population comprises neural cells that produce GABA.

4. The method of claim 3, wherein the neural cells are capable of producing GABA in vitro.

5. The method of claim 1, wherein the neural cells are capable of producing GABA following transplantation into a mammalian nervous system.

6. The method of claim 1, wherein the neural cells of the isolated population express two or more of ADAMTS5, ARX, ATRNL1, BMP3, CADPS, CALB2, CD200, CELSR3, CHRM4, CNTNAP4, CRABP1, CSMD3, CXCR4, CXCR7, DCLK2, DCX, DLX1, DLX2, DLX5, DLX6, DLX6-AS1, DSCAML1, ELAVL2, ELFN1, ENSG00000260391, EPHA5, ERBB4, ETS1, FAM5B, FAM65B, FNDC5, GAD1, GAD2, GNG2, GPD1, GRIA1, GRIA4, GRIK3, GRIN2B, HMP19, IGF1, INA, KALRN, KCNC2, KDM6B, KIF21B, L1CAM, LHFPL3, LHX6, LINC00340, LINC00599, MAF, MAFB, MAPT, MEF2C, MIAT, NCAM1, NKX2-1, NMNAT2, NPAS1, NRCAM, NRXN3, NXPH1, PDZRN3, PDZRN4, PIP5K1B, PLS3, PLXNA4, PNOC, PRLHR, PTPRB, PTPRR, RAI2, ROBO1, ROBO2, RP11-384F7.2, RPH3A, RP4-791M13.3, RUNX1T1, SCG3, SCRT1, SCRT2, SIAH3, SLC32A1, SLC6A1, SOX6, SP9, SRRM4, SST, ST8SIA5, STMN2, TAGLN3, THRB, TIAM1, TMEM2, TTC9B, VAX1, VSTM2A or WI2-1896O14.1.

7. The method of claim 1, further comprising differentiating said population of isolated cells into GABA-producing neurons.

8. The method of claim 1 wherein the neural cells of interest express the cell surface marker ATRNL1, CD200, CELSR3, CHRM4, CNTNAP4, CSMD3, CXCR4, CXCR7, DSCAML1, ELFN1, EPHA5, ERBB4, FAM5B, FAM65B, FNDC5, GRIA1, GRIA4, GRIK3, GRIN2B, KCNC2, L1CAM, LHFPL3, NCAM1, NRCAM, NRXN3, NXPH1, PLXNA4, PRLHR, PTPRB, PTPRR, ROBO1, ROBO2, SLC6A1, or TMEM2.

9. The method of claim 1, wherein the neural cells express PLEXINA4.

10. The method of claim 1, wherein the neural cells express CXCR4.

11. The method of claim 1, wherein the neural cells express CXCR7.

12. The method of claim 1, wherein the neural cells express ERBB4.

13. The method of claim 1, wherein the cells are isolated using a binding agent to a cell-surface marker of claim 8.

14. The method of claim 1, wherein the cells are isolated by decreasing the number of cells with low expression of one or more markers upregulated in neural cells of interest.

15. The method of claim 1, further comprising the step of cryopreserving the isolated neural cells.

16. The isolated neural cells of claim 1.

17. A method of generating a population of neural cells, comprising:
providing a population of pluripotent mammalian stem cells; and
differentiating the stem cells under conditions to obtain a culture of enriched neural cells with increased expression of two or more markers upregulated in neural cells of interest;
wherein the enriched cell population comprises neural cells.

18. The method of claim 1, wherein the cell population comprises neural cells capable of forming GABA-producing neurons.

19. The method of claim 17, wherein the cell population comprises neural cells that produce GABA.

20. The method of claim 17, wherein the neural cells are capable of producing GABA in vitro.

21. The method of claim 17, wherein the neural cells are capable of producing GABA following transplantation into a mammalian nervous system.

22. The method of claim 17, wherein the pluripotent mammalian stem cells are human pluripotent stem cells.

23. The method of claim 17, wherein the neural cells of the enriched population express two or more of ADAMTS5, ARX, ATRNL1, BMP3, CADPS, CALB2, CD200, CELSR3, CHRM4, CNTNAP4, CRABP1, CSMD3, CXCR4, CXCR7, DCLK2, DCX, DLX1, DLX2, DLX5, DLX6, DLX6-AS1, DSCAML1, ELAVL2, ELFN1, ENSG00000260391, EPHA5, ERBB4, ETS1, FAM5B, FAM65B, FNDC5, GAD1, GAD2, GNG2, GPD1, GRIA1, GRIA4, GRIK3, GRIN2B, HMP19, IGF1, INA, KALRN, KCNC2, KDM6B, KIF21B, L1CAM, LHFPL3, LHX6, LINC00340, LINC00599, MAF, MAFB, MAPT, MEF2C, MIAT, NCAM1, NKX2-1, NMNAT2, NPAS1, NRCAM, NRXN3, NXPH1, PDZRN3, PDZRN4, PIP5K1B, PLS3, PLXNA4, PNOC, PRLHR, PTPRB, PTPRR, RAI2, ROBO1, ROBO2, RP11-384F7.2, RPH3A, RP4-791M13.3, RUNX1T1, SCG3, SCRT1, SCRT2, SIAH3, SLC32A1, SLC6A1, SOX6, SP9, SRRM4, SST, ST8SIA5, STMN2, TAGLN3, THRB, TIAM1, TMEM2, TTC9B, VAX1, VSTM2A or WI2-1896O14.1.

24. The method of claim 17, further comprising differentiating said population of enriched cells into GABA-producing neurons.

25. The method of claim 17, further comprising isolating the neural cells of interest from the culture.

26. The method of claim 17, wherein the neural cells of interest express the cell surface marker ATRNL1, CD200, CELSR3, CHRM4, CNTNAP4, CSMD3, CXCR4, CXCR7, DSCAML1, ELFN1, EPHA5, ERBB4, FAM5B, FAM65B, FNDC5, GRIA1, GRIA4, GRIK3, GRIN2B, KCNC2, L1CAM, LHFPL3, NCAM1, NRCAM, NRXN3, NXPH1, PLXNA4, PRLHR, PTPRB, PTPRR, ROBO1, ROBO2, SLC6A1, or TMEM2.

27. The method of claim 17, wherein the neural cells express PLEXINA4.

28. The method of claim 17, wherein the neural cells express CXCR4.

29. The method of claim 17, wherein the neural cells express CXCR7.

30. The method of claim 17, wherein the neural cells express ERBB4.

31. The method of claim 25, wherein the cells are isolated using a binding agent to a cell-surface marker of claim 26.

32. The method of claim 17, wherein enriching the neural cells is achieved by decreasing the number of cells with low expression of one or more markers upregulated in neural cells of interest.

33. The method of claim 32, comprising the step of enriching said neural cells by depleting the cells expressing a cell-surface marker from the group of ATP1A2, BCAN, CD271, CD98, CNTFR, EGFR, FGFR1, FGFR2, FGFR3, GJA1, MLC1, NOTCH1, NOTCH3, PDGFRB, PDPN, PLXNA4, PROM1, PTPRZ1, SLC1A3, SLC1A5, TMEM158, or TTYH1.

34. The method of claim 17, further comprising the step of cryopreserving the enriched or isolated neural cells.

35. The enriched neural cells of claim 17.

36. A method of generating a population of neural cells, comprising:
providing mammalian cells; and
reprogramming the cells under conditions to obtain a culture of enriched neural cells with increased expression of two or more markers upregulated in neural cells of interest;
wherein the enriched cell population comprises neural cells.

37. The method of claim 36, wherein the cell population comprises neural cells capable of forming GABA-producing neurons.

38. The method of claim 36, wherein the cell population comprises neural cells that produce GABA.

39. The method of claim 36, wherein the neural cells are capable of producing GABA in vitro.

40. The method of claim 36, wherein the neural cells are capable of producing GABA following transplantation into a mammalian nervous system.

41. The method of claim 36, wherein the neural cells of the enriched population express two or more of ADAMTS5, ARX, ATRNL1, BMP3, CADPS, CALB2, CD200, CELSR3, CHRM4, CNTNAP4, CRABP1, CSMD3, CXCR4, CXCR7, DCLK2, DCX, DLX1, DLX2, DLX5, DLX6, DLX6-AS1, DSCAML1, ELAVL2, ELFN1, ENSG00000260391, EPHA5, ERBB4, ETS1, FAM5B, FAM65B, FNDC5, GAD1, GAD2, GNG2, GPD1, GRIA1, GRIA4, GRIK3, GRIN2B, HMP19, IGF1, INA, KALRN, KCNC2, KDM6B, KIF21B, L1CAM, LHFPL3, LHX6, LINC00340, LINC00599, MAF, MAFB, MAPT, MEF2C, MIAT, NCAM1, NKX2-1, NMNAT2, NPAS1, NRCAM, NRXN3, NXPH1, PDZRN3, PDZRN4, PIP5K1B, PLS3, PLXNA4, PNOC, PRLHR, PTPRB, PTPRR, RAI2, ROBO1, ROBO2, RP11-384F7.2, RPH3A, RP4-791M13.3, RUNX1T1, SCG3, SCRT1, SCRT2, SIAH3, SLC32A1, SLC6A1, SOX6, SP9, SRRM4, SST, ST8SIA5, STMN2, TAGLN3, THRB, TIAM1, TMEM2, TTC9B, VAX1, VSTM2A or WI2-1896O14.1.

42. The method of claim 36, further comprising differentiating said population of enriched cells into GABA-producing neurons.

43. The method of claim 36, further comprising isolating the neural cells of interest from the culture.

44. The method of claim 36, wherein the neural cells of interest express the cell surface marker ATRNL1, CD200, CELSR3, CHRM4, CNTNAP4, CSMD3, CXCR4, CXCR7, DSCAML1, ELFN1, EPHA5, ERBB4, FAM5B, FAM65B, FNDC5, GRIA1, GRIA4, GRIK3, GRIN2B, KCNC2, L1CAM, LHFPL3, NCAM1, NRCAM, NRXN3, NXPH1, PLEXINA4, PRLHR, PTPRB, PTPRR, ROBO1, ROBO2, SLC6A1, or TMEM2.

45. The method of claim 36, wherein the neural cells express PLEXINA4.

46. The method of claim 36, wherein the neural cells express CXCR4.

47. The method of claim 36, wherein the neural cells express CXCR7.

48. The method of claim 36, wherein the neural cells express ERBB4.

49. The method of claim 43, wherein the cells are isolated using a binding agent to a cell-surface marker of claim 44.

50. The method of claim 36, wherein enriching the neural cells is achieved by decreasing the number of cells with low expression of one or more markers upregulated in neural cells of interest.

51. The method of claim 50, comprising the step of enriching said neural cells by depleting the cells expressing a cell-surface marker from the group of ATP1A2, BCAN, CD271, CD98, CNTFR, EGFR, FGFR1, FGFR2, FGFR3, GJA1, MLC1, NOTCH1, NOTCH3, PDGFRB, PDPN, PLXNA4, PROM1, PTPRZ1, SLC1A3, SLC1A5, TMEM158, or TTYH1.

52. The method of claim 36, further comprising the step of cryopreserving the enriched neural cells.

53. The enriched neural cells of claim 36.
